(19) United States
(12) Patent Application Publication
(10) Pub. No.: US 2026/0091223 A1
(43) Pub. Date: Apr. 2, 2026

(54) PERCUTANEOUS METHOD FOR USING A SURGICAL TOOL TO DELIVER A DIRECTIONAL FIELD TO TREAT AN ANATOMICAL TARGET WITH STIMULATION

(71) Applicant: Micro-Leads, Inc., Somerville, MA (US)

(72) Inventors: Keith Angelino, Cambridge, MA (US); Chad Andresen, Boca Raton, FL (US); Bryan L. McLaughlin, Cambridge, MA (US); Mark Baldiswieler, Brookline, MA (US)

(21) Appl. No.: 19/342,450

(22) Filed: Sep. 26, 2025

Related U.S. Application Data
(60) Provisional application No. 63/699,686, filed on Sep. 26, 2024.

(51) Int. Cl. A61N 1/05; A61N 1/36; A61N 1/372

(52) U.S. Cl. CPC A61N 1/0553; A61N 1/36185; A61N 1/372

(57) ABSTRACT

A system delivers neuromodulation therapy. The system includes a surgical tool having a lumen and at least one directional stimulation feature, such as electrodes disposed on the outer surface, radial apertures, or a conductive stylet aligned with an aperture, configured to apply or assist in applying a directional field toward a neural target. The system further includes a permanent electrode lead having a directional electrode arrangement and an alignment feature that preserves the orientation of the lead relative to the surgical tool during deployment.

### PRIORITY

This patent application claims priority from provisional U.S. patent application No. 63/699,686, filed Sep. 26, 2024, entitled, “Percutaneous Method for Using a Surgical Tool to Deliver a Directional Field to Treat an Anatomical Target with Stimulation,” and naming Keith Angelino, Chad Andresen, Bryan Mclaughlin, and Mark Baldiswieler as inventors, the disclosure of which is incorporated herein, in its entirety, by reference.

### FIELD OF THE INVENTION

Illustrative embodiments of the invention generally relate to implantable devices with attached indwelling electrode leads for delivering nerve stimulation therapy or measuring physiological or neurological signals.

### BACKGROUND OF THE INVENTION

Indwelling medical devices are used to treat a wide range of disease conditions and include Spinal Cord Stimulation (SCS), Sacral Nerve Stimulation (SNS), deep brain stimulation, Peripheral Nerve Stimulation (PNS), Vagus Nerve Stimulation (VNS), Carotid Baroreceptor Stimulation (CBS), Cardiac Resynchronization Therapy, Cardiac Rhythm Management, and Injectable Monitoring, etc. Indwelling devices for interfacing with anatomical targets have included those requiring surgical placement (e.g., laminectomy paddle leads for spinal cord stimulation, surface paddles for the brain surface, nerve cuff leads for peripheral nerves) and some which may be placed using a catheter or through the bore of needle (e.g., percutaneous spinal cord stimulation leads or injected intramuscular wire electrodes).

### SUMMARY OF VARIOUS EMBODIMENTS

In accordance with one embodiment, a catheter is provided that is configured for use in neuromodulation therapy. The catheter includes a tubular body defining a lumen that can receive and deliver an implantable therapy electrode. Disposed on the tubular body is at least one directional stimulation feature, which is configured to assist in delivering a directional stimulation field in a desired angular orientation relative to the body of the catheter.

In various embodiments, the directional stimulation feature may be implemented in different ways. For example, it may take the form of a radial aperture extending through the catheter wall, one or more electrodes integrated on the catheter surface, or an aperture that exposes a conductive stylet within the lumen. The directional stimulation feature can also include a plurality of electrodes arranged circumferentially about the catheter, with each electrode being selectively activatable to generate a directional stimulation field, or a plurality of axially staggered apertures along the catheter body.

The catheter may further incorporate features to improve alignment and guidance. In some cases, fiducial markers, keyed handles, or radiopaque indicators are provided to ensure that a permanent electrode delivered through the lumen can be aligned with the established directional stimulation field. The directional field itself may exhibit a primary angular region having a magnitude that is at least 10-20% greater than a non-primary region, and in certain embodiments the field is asymmetric and spans less than 270° around the catheter body.

The catheter can be constructed from a variety of materials and designs. For instance, the tubular body may include a flexible polymer sheath with printed conductive traces forming the directional feature, or metallic electrodes embedded in or affixed to the catheter wall. In some embodiments, a rotatable stylet is positioned within the lumen, with one or more conductive segments alignable with apertures in the tubular body to produce the directional field. Alternatively, the directional feature may be embodied as a multiplexed electrode array operable with a stimulation control unit to selectively activate different subsets of electrodes.

The catheter is compatible with different types of therapy electrodes. In certain examples, the catheter is configured for the delivery of a segmented cylindrical electrode lead, while in other cases it is configured for the delivery of a self-expanding paddle electrode. The catheter may be used to stimulate a wide range of targets, including peripheral nerves, nerve plexuses, neural tissue, physiological tissue, or brain nuclei, using directional stimulation. Specific applications include intraoperative mapping of the hypoglossal nerve, pudendal nerve, or carotid sinus nerve.

A system is also provided for directional neuromodulation therapy, which includes the catheter described above in combination with an implantable therapy electrode that is deliverable through the catheter lumen. In this system, the therapy electrode can be positioned such that its directional field corresponds to the orientation of the directional stimulation field provided by the catheter, thereby enabling efficient alignment and accurate delivery of therapy.

In accordance with another embodiment, a system is provided for delivering neuromodulation therapy. The system includes a directional surgical tool having a lumen and configured to apply, or to assist in applying, a directional stimulation field. The directional stimulation field exhibits a primary field magnitude in a selected direction, which can be used to identify an optimal therapeutic orientation. The system further includes a permanent electrode lead that can be deployed through the lumen of the directional surgical tool in an orientation corresponding to the desirable stimulation direction identified during the procedure.

In some embodiments, the directional surgical tool may include structural or electrical features that provide or assist in directional stimulation. For example, the tool may include a plurality of radial apertures that direct the stimulation field, a plurality of electrodes disposed on its outer surface with a control mechanism to selectively activate different electrodes, or a conductive stylet positioned within the lumen with an exposed portion aligned to an aperture to generate the directional field.

The permanent electrode lead can also take different forms to achieve the desired therapeutic effect. In one embodiment, the lead is a self-expanding paddle electrode that transitions from a compressed state within the lumen to an expanded state once deployed, providing a hemispherical stimulation field. In another embodiment, the lead is a cylindrical design incorporating radially segmented electrodes to deliver directional stimulation.

To ensure that the permanent electrode lead maintains its therapeutic orientation, the system may include alignment features such as fiducial markers, keyed connectors, or mechanical coupling elements that secure the relative orientation of the catheter and the lead during deployment. In addition, the system may further comprise a control system configured to monitor physiological responses in real time and to assist in identifying the most desirable direction for stimulation.

Certain embodiments place emphasis on the self-expanding paddle electrode. The paddle may be biased to expand into a planar configuration when deployed, producing a hemispherical directional stimulation field. This configuration can provide several advantages, including greater energy efficiency by reducing required stimulation amplitudes relative to cylindrical electrodes, selective targeting of on-target nerves while avoiding off-target activation, and improved anti-migration stability by anchoring within tissue following expansion.

Alternatively, or in combination, the permanent electrode lead may comprise a cylindrical lead with multiple radially segmented electrodes arranged to deliver a controlled directional stimulation field, providing additional flexibility for tailoring therapy to patient-specific anatomy and clinical needs.

In accordance with yet another embodiment, illustrative embodiments provide a system having a directional catheter and a permanent electrode lead. The method of use applies a first directional stimulation field in a first orientation from the catheter, then applying a second directional field in a different orientation, and subsequently deploying the permanent electrode lead through the lumen of the catheter in alignment with the desired orientation. In some embodiments, the permanent electrode lead is aligned with the chosen direction using features such as radiopaque markers, visual fiducials, or keyed loading mechanisms that maintain rotational consistency between the surgical tool and the lead. In certain implementations, the first directional field is discontinued once the second, optimal orientation is identified.

A system delivers neuromodulation therapy. The system includes a surgical tool having a lumen and at least one directional stimulation feature, such as electrodes disposed on the outer surface, radial apertures, or a conductive stylet aligned with an aperture, configured to apply or assist in applying a directional field toward a neural target. The system further includes a permanent electrode lead having a directional electrode arrangement and an alignment feature that preserves the orientation of the lead relative to the surgical tool during deployment.

In yet another embodiment, a method delivers neuromodulation therapy. The method includes inserting a surgical tool with a directional stimulation feature proximate to a neural target, applying an asymmetric directional field having a primary field magnitude in a selected orientation, and monitoring one or more physiological responses to determine the most desirable therapeutic direction. A permanent electrode lead is then deployed through the lumen of the surgical tool in that orientation. The method may further involve adjusting the orientation of the directional field prior to deployment and comparing physiological responses to confirm the optimal direction. In some embodiments, the orientation of the surgical tool is recorded relative to an anatomical reference or fiducial marker. The directional field may be defined as having a primary magnitude at least 10% greater in one direction than in any other.

The surgical tool may be embodied as a catheter with radial apertures, an outer surface array of electrodes, or a conductive stylet aligned with one or more apertures. The permanent electrode lead may be a self-expanding paddle electrode that transitions from a compressed state within the lumen to an expanded hemispherical configuration, or a cylindrical lead with radially segmented electrodes for delivering controlled directional fields. Physiological responses used to determine therapy direction may include electromyography, evoked compound action potentials, airway patency, muscle movement, or blood oxygen saturation. In some cases, these responses are automatically analyzed by a control system to identify the optimal therapeutic orientation.

The desirable therapeutic direction is generally identified when stimulation produces a physiological response indicative of a positive therapeutic effect while avoiding adverse responses. After the permanent electrode lead is deployed, the surgical tool is withdrawn, leaving the electrode in place to provide long-term directional neuromodulation therapy.

In illustrative embodiments, a method identifies a desirable stimulation direction and deploying a therapy electrode. The method includes positioning a catheter having a lumen and at least one directional stimulation feature proximate to a neural target. A directional stimulation field is applied from the catheter in a first orientation, and feedback is received that is indicative of whether that orientation corresponds to a desirable therapeutic effect. The orientation of the stimulation field may then be adjusted, and the applying and feedback steps repeated until the desirable direction is identified. Once the optimal orientation is determined, an electrode lead is deployed through the lumen of the catheter in alignment with that direction, after which the catheter is withdrawn while leaving the therapy electrode lead in place to deliver long-term stimulation.

In some illustrative embodiments, the electrode lead deployed through the catheter may be a self-expanding paddle electrode that transitions from a compressed state within the catheter lumen to an expanded paddle configuration when released. The paddle electrode may be biased to expand into a generally planar shape that produces a hemispherical directional stimulation field, and in certain examples may include features that enhance energy efficiency by lowering required stimulation amplitudes relative to cylindrical electrodes, or that improve anchoring and anti-migration stability after expansion. In other embodiments, the electrode lead may take the form of a cylindrical device with radially segmented electrodes configured to deliver controlled directional stimulation. Alignment of the electrode lead with the identified stimulation direction can be facilitated by radiopaque markers, fiducial indicators, or keyed loading mechanisms that maintain rotational consistency between the catheter and the electrode. Feedback used to determine the desirable stimulation direction may include electrical signals recorded from sensors, measurements of mechanical displacement, indicators of field coupling, or perceptible outputs such as visible displacement of a motion element or audible responses associated with the target tissue.

In illustrative embodiments, a method of delivering a neuromodulation therapy electrode includes introducing a catheter into a patient, where the catheter defines a lumen configured to receive the therapy electrode and incorporates at least one directional stimulation feature. The directional stimulation feature is operated to apply, or to assist in applying, a directional stimulation field that enables identification of a desirable stimulation orientation relative to a neural target. Once the optimal orientation is determined, the therapy electrode is advanced through the lumen of the catheter in that same orientation, after which the catheter is withdrawn while the therapy electrode remains implanted to deliver long-term stimulation in the desirable orientation.

In some illustrative embodiments, the feedback used to identify a desirable stimulation orientation may comprise a visible physiological response such as a muscle twitch or movement, or verbal feedback provided directly by the patient.

Additional embodiments relate to the structure of the therapy electrode lead itself. The lead may include a longitudinal body with a substrate and at least one electrode configured to deliver or record an electrical signal. The electrode may be formed with a nonuniform thickness or a discontinuity, and the lead may be designed to transition between a compressed configuration and an extended configuration. The substrate is normally biased toward the extended configuration, such that when the lead is compressed, the electrode is correspondingly compressed, and upon release, the substrate urges the electrode back toward the extended state. In some examples, the electrode includes a thickness ranging from about 2 microns to about 200 microns.

Methods of use are also contemplated in which the electrode lead is directed into a compressed configuration, thereby compressing an electrode having a nonuniform thickness or discontinuity. The electrode is configured to deliver an electrical signal and may have a thickness between about 2 and 200 microns. The method further includes transitioning the lead from the compressed configuration to an extended configuration by relying on the bias of the substrate, which extends the electrode to its operative state.

Illustrative embodiments of the invention are implemented as a computer program product having a computer usable medium with computer readable program code thereon. The computer readable code may be read and utilized by a computer system in accordance with conventional processes.

### BRIEF DESCRIPTION OF THE DRAWINGS

The patent or application file contains at least one drawing executed in color. Copies of this patent with color drawing(s) will be provided by the Patent and Trademark Office upon request and payment of necessary fee.

Those skilled in the art should more fully appreciate advantages of various embodiments of the invention from the following “Description of Illustrative Embodiments,” discussed with reference to the drawings summarized immediately below.

FIGS. 1A-1C schematically show different types of stimulation fields created by electrode geometries in accordance with illustrative embodiments.

FIG. 1D schematically shows views of a percutaneous electrode with ring electrodes delivering an omni-directional stimulation field in accordance with illustrative embodiments.

FIGS. 2A-2B schematically show directional stimulation fields in accordance with illustrative embodiments.

FIG. 3A schematically shows an omnidirectional stimulation field that treats multiple anatomical targets simultaneously in accordance with illustrative embodiments of the invention.

FIG. 3B schematically shows a directional stimulation field treating one target while avoiding other targets in accordance with illustrative embodiments of the invention.

FIG. 4 shows a process of delivering targeted therapy in accordance with illustrative embodiments of the invention.

FIG. 5A schematically shows a percutaneous electrode with segmented electrodes configured to deliver one or more directional stimulation fields in accordance with illustrative embodiments.

FIG. 5B schematically shows a percutaneous self-expanding paddle electrode configured to deliver one or more directional stimulation fields in accordance with illustrative embodiments.

FIG. 6 schematically shows a surgical tool for delivery of directional stimulation fields having a guidewire in accordance with illustrative embodiments.

FIG. 7 schematically shows a surgical tool with apertures configured to assist in the application of a directional stimulation field in accordance with illustrative embodiments.

FIG. 8 schematically shows the surgical tool with aperture features for delivering directional fields accordance with illustrative embodiments.

FIG. 9 schematically shows marker features on a paddle (e.g., self-expanding paddle rolled inside the cannula) and the surgical tool to enable the position and directional field direction to be desirably oriented in accordance with illustrative embodiments.

FIG. 10 schematically shows a canula applying directional fields using electrodes coupled with the cannula in accordance with illustrative embodiments.

FIG. 11 schematically shows a dilator tool that aids in the application of a directional field in accordance with illustrative embodiments.

FIG. 12 shows a method of identifying and delivering a directional field for an optimal therapeutic benefit in accordance with illustrative embodiments.

FIG. 13 schematically shows an example of a system for applying a directional stimulation field with input parameters and example physiology parameters to maximize on-target physiological effects while minimizing off-target physiological effects in accordance with illustrative embodiments.

FIG. 14 schematically shows a negative determination of net therapeutic benefit from physiological observations after a directional stimulation is applied in accordance with illustrative embodiments.

FIG. 15 schematically shows a positive determination of net therapeutic benefit from physiological observations after a directional stimulation is applied in accordance with illustrative embodiments.

FIG. 16 shows a method of identifying a directional field to deploy a self-expanding paddle for net therapeutic benefit in accordance with illustrative embodiments.

FIG. 17 shows an automated method for identifying the directional field orientation which provides a net therapeutic benefit

FIG. 18 schematically shows treatment steps for applying a directional stimulation field for net therapeutic benefit in accordance with illustrative embodiments.

FIG. 19 schematically shows a surgical tool with one or more features to facilitate the application of directional stimulation in accordance with illustrative embodiments.

FIG. 20 schematically shows the surgical tool aids in the application of a directional field to an anatomical target and the un-successful achievement of a net therapeutic effect in accordance with illustrative embodiments.

FIG. 21 schematically shows the surgical tool aids in the application of a directional field to an anatomical target and the successful achievement of a net therapeutic effect in accordance with illustrative embodiments.

FIG. 22 schematically shows the surgical tool aids an implantable electrode in the application of multiple directional fields to an anatomical target and the evaluations of a net therapeutic benefit in accordance with illustrative embodiments.

FIG. 23 schematically shows the self-expanding paddle is aligned with a surgical tool relative to the anatomy to facilitate the application of a directional field in accordance with illustrative embodiments.

FIG. 24 schematically shows the self-expanding paddle is mechanically coupled with a surgical tool and advanced to the desired anatomical target in accordance with illustrative embodiments.

FIG. 25 schematically shows the self-expanding paddle is deployed at the anatomical target to deliver a directional field in the optimal direction to provide a net therapeutic benefit in accordance with illustrative embodiments.

FIG. 26 schematically shows the surgical tool is removed from the indwelling electrode in accordance with illustrative embodiments.

FIG. 27 schematically shows one or more directional fields are delivered to determine and provide a desired net therapeutic benefit in accordance with illustrative embodiments.

FIG. 28 schematically shows selective directional stimulation of the pudendal nerve (on-target) while avoiding unintended activation of the adjacent sciatic nerve (off-target) in accordance with illustrative embodiments.

FIG. 29 schematically shows directional stimulation of the carotid sinus nerve to modulate baroreceptor activity for precise blood pressure regulation while minimizing off-target effects in accordance with illustrative embodiments.

FIG. 30 schematically shows details of controller of the system configured in accordance with illustrative embodiments of the invention.

FIGS. 31A-31B are cross-sectional views schematically showing an electrophysiology lead in accordance with various embodiments.

FIG. 32 schematically shows cross-sectional view of the electrophysiology lead in accordance with various embodiments.

FIG. 33 schematically shows cross-sectional view of substrates of the electrophysiology leads in accordance with various embodiments.

FIGS. 34A-34E schematically show substrate mechanical anchors in accordance with various embodiments.

FIGS. 35A-35D show a sequence for decompressing an implanted lead in accordance with various embodiments.

### DESCRIPTION OF ILLUSTRATIVE EMBODIMENTS

In illustrative embodiments, a method and system for minimally invasive, directional neuromodulation therapy eliminate the need for open surgery while ensuring precise targeting of neural structures. Illustrative embodiments provide a directional mapping workflow using a temporary percutaneous surgical tool (e.g., catheter, cannula, or introducer) capable of generating or aiding a directional stimulation field through integrated electrodes, apertures, or a conductive stylet. This tool allows clinicians to identify a desirable stimulation direction intraoperatively by observing physiological responses (e.g., EMG, airway patency, muscle activation) and then deploys a directional electrode, such as a segmented cylindrical lead or a self-expanding paddle, in that same orientation without additional manipulation. The desirable stimulation direction maximizes on-target therapeutic effect, minimizes off-target effects, and may be confirmed by physiological feedback

Unlike conventional percutaneous leads that deliver omnidirectional fields or paddle leads that require open surgery, illustrative embodiments advantageously combine percutaneous delivery with paddle-like directional benefits, including energy efficiency, reduced off-target activation, and anti-migration stability. The system also incorporates orientation alignment features (e.g., fiducials, keyed handles) to maintain directional consistency between the mapping tool and the implanted electrode. Various embodiments include automated closed-loop algorithms for real-time optimization and multiple catheter configurations (printed electrodes, staggered apertures, or rotatable stylets) to achieve directional stimulation.

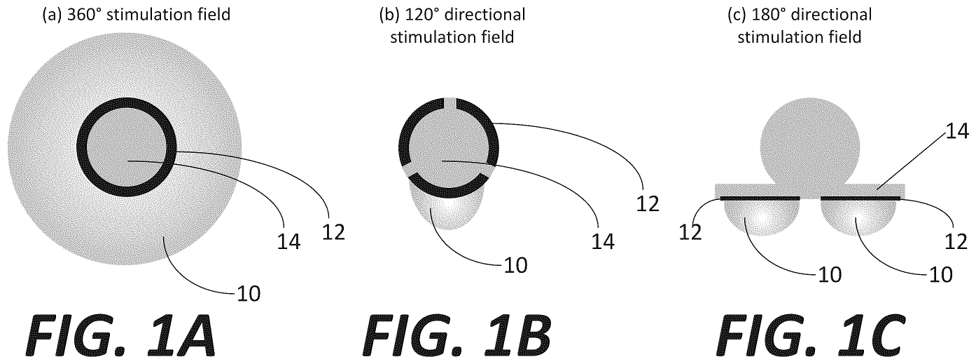

FIG. 1 illustrates representative stimulation field 10 distributions generated by different electrode geometries. Implantable leads 14 typically include surgical-incision-placed or needle-placed leads which have different geometries to provide different treatment types. Percutaneous leads 14 are placed without an open surgical exposure, and they are widely used for stimulating or recording in the spinal cord, brain, peripheral nerves, or other physiological targets. Because percutaneous leads 14 have a tubular or cylindrical geometry (See, for example, FIG. 1D) and utilize ring-shaped cylindrical electrode contacts 12, they may be easily delivered through a catheter, cannula, needle, or delivery sheath. Other percutaneous leads 14 may include barbed wires forming a hook electrode delivered through a needle.

The methods described herein may use surgical-tools (e.g., catheter, needle, guidewire) and indwelling electrodes to deliver desirable neurostimulation therapy for a net positive therapeutic benefit. More specifically, the method delivers indwelling devices which desirably couple directional stimulation fields into anatomical targets (e.g., a peripheral nerve, branches, nucleus, or surrounding muscles) using a directional stimulation field 10. Specifically, illustrative embodiments use a surgical tool to aid and facilitate the application of a directional stimulation therapy field to focus therapy to an anatomical target to maximize therapeutic benefit. The method observes a physiological response to the application of the directional field 10. The method tests one or more directional field distributions to observe, identify, and obtain a desired net therapeutic physiological benefit. The method then removes the surgical tool and uses the indwelling electrode to provide a directional stimulation field to provide the desirable net therapeutic benefit.

The method generally applies to nerve stimulation or neuromodulation where directional field stimulation is used to stimulate or record from neural tissue (e.g. peripheral nerves, spinal cord, brain, or adjacent muscles) Specific embodiments relate to peripheral nerve stimulation and arborization of nerve branches. Other embodiments relate to specific neural anatomical structures (e.g., subthalamic nucleus of the brain, hypoglossal nerve, or pelvic plexus) Using a directional stimulation field, the applied therapy field may be narrowed or spatially oriented to deliver treatment to specific anatomical targets to provide a net therapeutic benefit. A net therapeutic benefit may result if a directional field is used to deliver treatment to a desired target while reducing or preventing the field from being delivered to adjacent anatomies which may produce off-target effects of stimulation and reduce therapy benefits.

Cylindrical leads 14 for neuromodulation generally have a small diameter (e.g., less than 2.5 mm) and typically utilize cylindrical ring electrode contacts 12 for stimulation or recording. Due to the diameter constraints required for needle insertion, the electric field penetration by a cylindrical lead 14 into tissue limits the treatment volume in a medio-lateral direction to less than 2.5 mm. The recording or stimulation field of a cylindrical ring electrode contact 12 is typically a spherical or cylindrical diffused volume, as shown in FIG. 1A. FIG. 1A illustrates a cylindrical lead 14 incorporating a circumferential ring electrode that produces a substantially omnidirectional stimulation field. The generated field extends radially outward in all directions about the lead body to form an approximately 360° spherical or cylindrical distribution. While such omnidirectional fields are suitable for broad activation of surrounding tissue, they undesirably lack selectivity and may stimulate both desired target tissue and adjacent, off-target structures, thereby reducing therapeutic benefit and producing undesirable side effects.

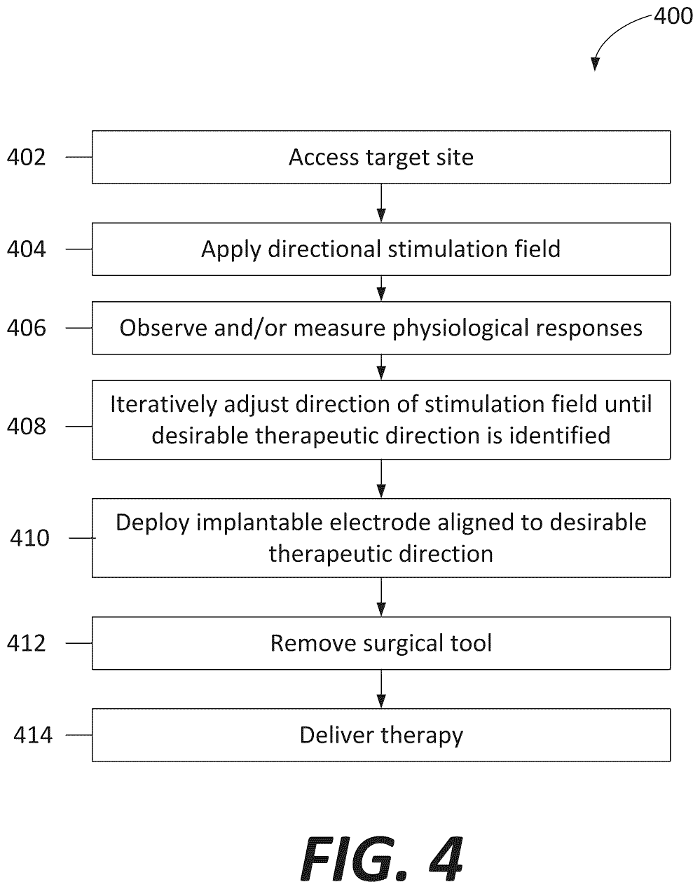

To improve selectivity, a directional cylindrical electrode may be configured with multiple segmented contacts 12 arranged radially around an axis to allow stimulation to be delivered primarily in one or more angular directions, as shown in FIG. 1(b) and FIG. 5(a). When one or more of these segments are activated, the resulting field 10 distribution becomes asymmetric, extending primarily over a restricted angular arc (for example, approximately 90° to 150°, shown here as approximately) 120°. This configuration enables a directional stimulation field 10 to be delivered with at least a 10-20% higher field magnitude in the primary angular region relative to non-primary regions.

The directional stimulation field 10 refers to a stimulation field that is asymmetric in angular distribution around an electrode such that one or more angular regions exhibit a field magnitude greater than other angular regions. In some embodiments, the primary region may correspond to a single angular lobe of enhanced field magnitude relative to adjacent non-preferred regions. In other embodiments, two or more primary regions of similar magnitude may exist, each of which exhibits a field magnitude greater than at least one other region. Thus, a directional field may include one or multiple primary regions, provided that the field distribution is not uniform across all angular directions.

By steering current through selective activation of electrode segments, the locus of maximum field intensity can be shifted circumferentially to align with the intended neural target while reducing activation of adjacent tissue. While directional cylindrical electrodes provide improved directional control compared to omnidirectional electrodes, the small diameter of percutaneous cylindrical leads (typically less than 2.5 mm, and in some embodiments less than 1.5 mm) still limits the stimulation penetration depth and confines the treatable tissue volume to a relatively small cross-sectional area.

FIG. 1C illustrates a paddle electrode geometry in which one surface of the lead carries electrode contacts while the opposite surface is insulated. This configuration produces a hemispherical stimulation field distribution extending directionally away from the active surface, typically spanning approximately 180°. By confining the field to a single hemisphere, the paddle electrode primarily couples stimulation energy to the targeted anatomical region while insulating tissue located behind the electrode. Paddle electrodes therefore exhibit improved energy efficiency, with certain embodiments achieving up to a 50% reduction in stimulation amplitude and a four-fold reduction in energy usage relative to omnidirectional cylindrical electrodes for equivalent therapeutic effect. Additionally, because the paddle lead 14 presents a broader surface area, it can engage a larger volume of neural tissue, such as a peripheral nerve trunk, nerve plexus, or spinal cord segment, while simultaneously localizing stimulation to on-target regions. This directional coupling improves therapy outcomes while reducing off-target effects.

Surface leads 14 (e.g., paddles) are commonly used for diagnosis or treatment of neurological conditions where there is a need to capture physiological signals or deliver stimulation across a large anatomical surface area with greater specificity (e.g., across the full width of the spinal cord and multiple vertebral segments for pain treatment, or over a cortical region of the brain for epilepsy focus identification). Surface paddle leads 14 offer many therapeutic advantages over smaller percutaneous leads, though current paddles require open surgical placement.

A paddle 14 provides a larger surface treatment area, usually with many electrode contacts 12 patterned over the active surface. A surface paddle lead 14 captures a large area for recording or stimulation. Surface leads 14 are typically placed from an open surgical exposure, allowing for suitable visualization of the desired treatment area and enabling the insertion of a larger implant. Paddle leads 14 have a larger treatment area (2 mm×50 mm, 8 mm×50, 50×50 mm) than percutaneous leads which allow for the treatment of complex nerve targets spanning a wider volume including the spinal cord, sciatic nerve, or nerve plexuses.

Surface leads 14 are typically insulated on one side with electrode contacts 12 on the active surface, enabling the stimulation field 10 to be focused in a particular direction, orientation, or distribution. The electrode contacts 12 of a flat paddle 14 provide a directional treatment field with approximately a 180° hemispherical distribution from an insulated substrate (such as in FIG. 1c).

A directional stimulation field 10 is a stimulation field that is asymmetric in direction, generally treating a radial angle less than 270° degrees. FIGS. 1A-1C depict multiple therapeutic treatment fields including a (1) 360° omnidirectional (non-directional) stimulation field, (2) a 120° directional stimulation field, and (3) a 180° directional stimulation field. While many field directional field patterns are possible, these illustrate patterns from common electrode geometries.

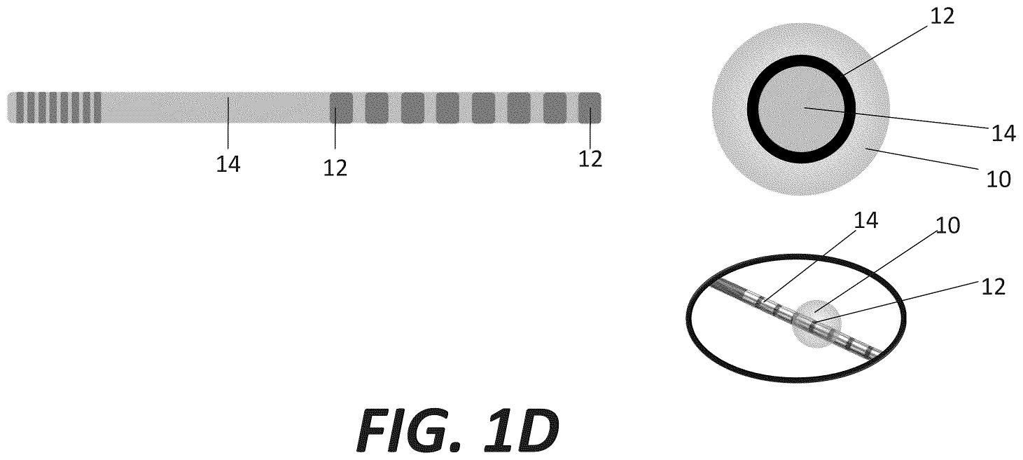

FIGS. 2A-2B schematically show examples of directional stimulation field patterns. A directional stimulation field 10 as shown in FIGS. 2A-2B normally has an asymmetric field distribution, with a primarily stimulation field magnitude in a particular direction. In the primary direction, the stimulation field 10 magnitude (e.g., electrical field intensity/density) is at least 10-20% greater than any other region or direction.

FIG. 2A shows three stimulation regions, e.g., two regions with a stimulation field magnitude A and primary region 10 with a directional field magnitude of 1.2*A. In a similar fashion, a stimulation field 10 may be applied omnidirectionally (FIG. 2B) at a field magnitude A, while overlapping a directional field pattern with a magnitude of 1.2*A. The primary directional field 10 produces a locus with greater therapeutic coupling than the other stimulated regions. Those skilled in the art will recognize that directional stimulation fields may be used to steer current or voltage potentials to change the direction, orientation, or position of the primary field locus or distribution in order to provide an desirable therapeutic response.

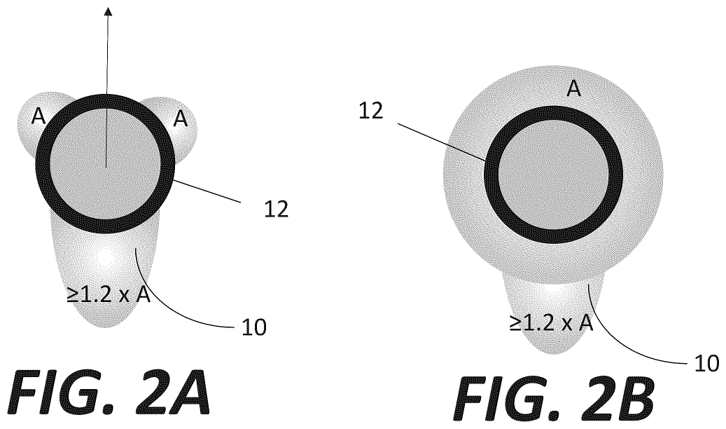

FIG. 3A shows an omnidirectional stimulation field 10 generated by a cylindrical lead positioned adjacent to three peripheral nerve branches PN. Because the applied field 10 is substantially uniform and extends radially in all directions about the electrode, each of the three peripheral nerves PN receives stimulation. In the example shown, the intended therapeutic target is the lower nerve branch (PN Target); however, the omnidirectional 360° field distribution (represented by the dotted line) simultaneously stimulates the two adjacent nerve branches, resulting in off-target activation. Such non-selective stimulation may undesirably produce adverse physiological responses, limit therapeutic efficacy, and reduce overall net benefit of the therapy.

FIG. 3B depicts a directional paddle electrode positioned adjacent to three peripheral nerve branches. In contrast to the omnidirectional configuration of FIG. 3A, the paddle 14 electrode generates a directional stimulation field 10 (represented by the dotted line) that extends substantially hemispherically (approximately) 180° away from the active electrode surface while insulating tissue located behind the paddle. The directional field 10 is desirably coupled to the desired target nerve branch PN Target located below the electrode while minimizing or preventing stimulation of the two adjacent nerve branches. By focusing the stimulation field 10 in a specific angular direction, the paddle 14 electrode provides enhanced selectivity, reduces off-target activation, and improves the delivery of on-target therapy.

Because the stimulation field 10 of the paddle 14 electrode is limited to a hemisphere, the paddle lead 14 can couple energy more efficiently in one direction to a peripheral nerve or spinal cord target than a cylindrical field. This efficiency can, in some cases, result in a 50% reduction in stimulation amplitude and a 4× lower use of stimulus energy. Furthermore, because the stimulation field can be pointed at the target anatomy while insulating the surrounding tissue, paddle leads 14 can often avoid undesirable stimulation of surrounding tissues (e.g., muscles or adjacent nerves) that is a common adverse event with percutaneous leads 14.

An additional benefit of paddle leads 14 is anti-migration properties. Whereas cylindrical leads 14 must be smooth to enable needle-based delivery, they are also free to move, slide, and piston in the tissue. By contrast, paddle leads 14 have a larger volume and geometry which more strongly anchors within the biological tissue. Paddles 14 provide a migration-resistant and more stable stimulation interface to a peripheral nerve or spinal cord.

Despite these advantages, paddle leads 14 necessitate an open surgical incision for placement under general anesthesia which carries a greater associated risk of surgical complications and post-surgical site pain.

### Peripheral Nerve Branches

Neuromodulation of peripheral nerves has typically required the use of an open incision and surgical dissection to apply a nerve cuff electrode circumferentially around the nerve to deliver therapy. The process of surgically wrapping the nerve cuff electrode typically relies upon identifying the nerve, placing the cuff, and testing the impact of stimulation on the nerve during an open incision procedure. In conventional surgical procedures, the nerve is identified visually or using electrical stimulation (e.g., using a Neural Integrity Monitoring System or other stimulator). The desired nerve, or the desired branches of the nerve can be tested and/or surgically separated or isolated to determine the desirable branches for placement of the stimulating nerve cuff. This process of surgically isolating nerve branches is particularly important for anatomies where nerves may distribute into multiple discrete branches, arborizations, or plexuses. By selectively isolating nerve branches, stimulation can be delivered to provide a therapeutic effect of one or more discrete branch(es) while also avoiding undesired branch(es) which produce off-target effects. Those skilled in the art will recognize complex nerve anatomies include, but are not limited to, the carotid sinus nerve (CSN), the hypoglossal nerve (HGN), pelvic plexus, sympathetic chain, ansa cervicalis, and others.

Peripheral nerves often bifurcate into multiple branches in an arborization arrangement, particularly as the nerve approaches the end organ (e.g., muscle, organ, bladder, etc.) For example, the HGN branches into the genioglossus, the HGN forms 3D arborization of fibers and branches. In a similar fashion, other peripheral nerves have similar branching including the glossopharyngeal, carotid sinus, pudendal, etc., Because the arborized branches of the nerve may correspond to on-target or off-target effects, the branches must be selected individually or in a programmatic fashion to desirably deliver therapy.

Those skilled in the art may recognize that the use of multi-contact electrode adjacent to a peripheral nerve, arborization, or complex nerve structure may provide the opportunity to localize a directional stimulation field to one region without delivering stimulation field to another area. Directional electrodes which pin-point stimulation in a particular direction with multiple locations while insulating other directions can be used to focus and orient stimulation desirably towards a nerve trunk or its arborized branches. For example, use of novel features of an electrode (e.g., multiple contacts or directionally-orientated electrodes) may allow one to deliver a directional stimulation field to treat therapeutic nerve branches while avoiding off-target branches. A correct stimulation field can improve the accuracy and effectiveness of the therapy.

FIG. 3A depicts the cross-section of an omnidirectional stimulation field applied between three peripheral nerve branches. When a uniform 360° field is applied, each of three peripheral nerves receives stimulation. As per the figure, the desired peripheral nerve target was located on the bottom, but all of the peripheral nerves were stimulated when only stimulation of the bottom target peripheral nerve was desired. In FIG. 3B, a directional paddle electrode 14 is deployed adjacent to three peripheral nerve branches. The directional field 10 focuses stimulation on the peripheral nerve target while insulating the peripheral nerves behind the electrode from stimulation. In this manner the 180° directional field distribution improves the delivery of on-target therapy versus off-target side-effects.

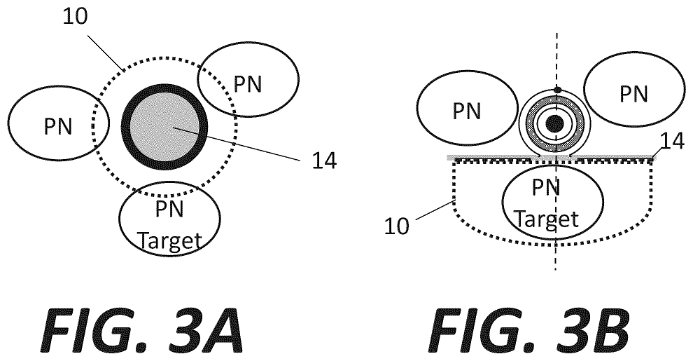

FIG. 4 shows a process of delivering targeted therapy in accordance with illustrative embodiments of the invention. It should be noted that this method is substantially simplified from a longer process that may normally be used. Accordingly, the method shown in FIG. 4 may have many other steps that those skilled in the art likely would use. In addition, some of the steps may be performed in a different order than that shown, or at the same time. Furthermore, some of these steps may be optional in some embodiments. Accordingly, the process 400 is merely exemplary of one process in accordance with illustrative embodiments of the invention. Those skilled in the art therefore can modify the process as appropriate.

Illustrative embodiments provide a method of inserting, advancing, orienting, and optimizing a directional therapeutic stimulation field of neurological tissue for a desired net therapeutic benefit. The method of delivering a directional stimulation field advantageously: (1) ensures that on-target physiological effects are maximized and off-target effects are minimized, and (2) allows the physician to rapidly determine the desirable stimulation field direction and implant an indwelling electrode to deliver a directional stimulation field for long-term net therapeutic benefit.

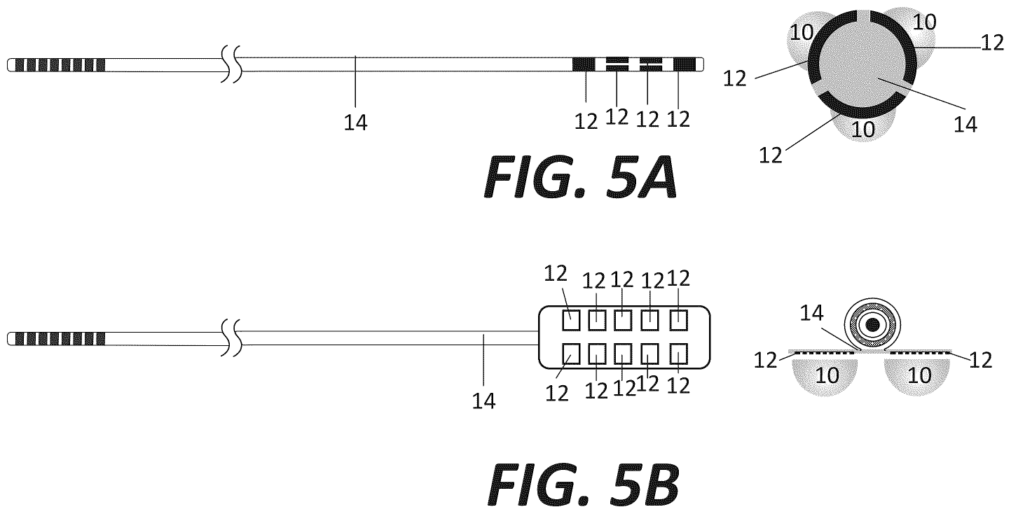

The method begins at step 402 by introducing a surgical system including a surgical tool (e.g., surgical tool 20 in FIG. 6) to access the desired anatomical region for therapy. The surgical tool may include, without limitation, a catheter, needle, cannula, introducer, guidewire, dilator, or stylet. The tool is advanced through the patient's tissue to a position adjacent to a target neural structure, such as a peripheral nerve, nerve branch, nerve plexus, spinal cord region, or brain nucleus. The tool may be guided into position using anatomical landmarks, imaging techniques (e.g., fluoroscopy, ultrasound, CT, or MRI), or fiducial markers associated with the electrode or surgical tool. By positioning the surgical tool near the intended target, the method enables delivery of a directional stimulation field in proximity to the anatomy of interest while minimizing the need for extensive surgical exposure.

At step 404, the method applies a directional stimulation field 10. The directional stimulation field 10 is applied intraoperatively through the surgical tool or through a partially-deployed indwelling electrode. In some embodiments, one or more stimulation electrodes may be integrated with the surgical tool, such as on the outer surface of a catheter or cannula, on a guidewire, or on a conductive stylet positioned within the tool. In other embodiments, a stimulating element may be advanced within the tool, or a partially-deployed paddle or cylindrical electrode may be energized to deliver a test field. In various embodiments, the stimulation field is configured to be asymmetric in angular distribution, such that one or more primary stimulation regions exhibit a field magnitude that is greater than surrounding secondary regions. In certain embodiments, the primary region has a field intensity at least 10-20% higher than secondary regions, for example producing a magnitude of approximately 1.2× A relative to a baseline field magnitude A. This directional field is oriented toward the suspected target anatomy, enabling localized testing of on-target versus off-target therapeutic effects. By delivering a controlled directional stimulation field intraoperatively, the method facilitates evaluation of the therapeutic response before final implantation of the indwelling electrode.

In illustrative embodiments, the catheter may apply or assist in applying a directional stimulation field. When the catheter itself applies the field, the catheter body incorporates active stimulating structures. For example, electrodes may be printed, deposited, or otherwise integrated on the catheter wall, such that energization of the electrodes produces a stimulation field that is asymmetric in angular distribution. In other embodiments, circumferentially distributed electrode segments are positioned around the catheter body and selectively activated to steer the field in a chosen direction. In still other examples, a portion of the catheter wall may be fabricated from a conductive material while the remainder of the wall is insulated, such that stimulation energy preferentially couples in a single angular orientation.

In alternative embodiments, the catheter does not itself generate the stimulation field but instead assists in applying the field produced by another stimulating element, such as a stylet, introducer, or indwelling electrode placed within the lumen. In one embodiment, the catheter wall includes one or more apertures or windows that expose the stimulating element inside the lumen, thereby directing the field outward through the opening in a defined orientation. In another embodiment, the catheter incorporates dielectric or shielding materials along portions of its circumference, blocking current spread in some directions while allowing coupling in others. In further embodiments, the catheter includes staggered apertures or partial circumferential windows such that a conductive stylet or the permanent electrode (e.g., the paddle electrode 14) within the lumen can be rotated to selectively align with different windows, enabling directional stimulation without requiring repositioning of the entire catheter. In yet another example, the catheter assists by providing fiducials, keyed handles, or radiopaque markers that maintain the angular orientation between the mapping field and the subsequently deployed therapy electrode.

Collectively, these embodiments are referred to herein as including a directional stimulation feature. As used in this application, a directional stimulation feature encompasses any structure or element of the catheter that generates, shapes, or enables the delivery of a directional stimulation field. This definition expressly covers features that are on, within, or defined by the catheter body itself, including but not limited to surface electrodes, embedded conductors, apertures, shielding regions, stylet interfaces, and marker-based alignment features. By defining the catheter broadly in terms of a directional stimulation feature, illustrative embodiments both catheters that actively apply stimulation and catheters that assist in directing stimulation provided by another component.

At step 406, after delivery of the directional stimulation field, the method includes observing and measuring one or more physiological responses to stimulation in order to distinguish therapeutic on-target effects from undesired off-target effects. In some embodiments, the therapeutic response may include airway opening, tongue protrusion, muscle recruitment, reduction in pain, modulation of heart rate, or changes in inflammatory biomarkers. In other embodiments, the therapeutic response may be detected using quantitative physiological measurements such as electromyography (EMG), evoked compound action potentials (ECAPs), sensory perception thresholds, plethysmography, endoscopy, or blood oxygenation levels. These feedback mechanisms allow the clinician to verify that stimulation delivered through the primary stimulation region is producing the desired physiological effect, while identifying any secondary regions that may activate adjacent, non-target tissues. By observing the relative presence or absence of therapeutic benefit across different stimulation regions, the method enables accurate determination of the initial effectiveness of the directional stimulation field prior to final implantation of the electrode.

At step 408, the method iteratively adjusts the orientation or configuration of the directional stimulation field (e.g., the primary stimulation field) until desirable therapeutic direction is identified. In some embodiments, the surgical tool may be rotated, translated, or otherwise repositioned to alter the angular orientation of the stimulation field relative to the target anatomy. In other embodiments, different electrode contacts may be selectively activated, alone or in combination, to generate one or more primary stimulation regions oriented in different directions. By systematically varying the stimulation field, the clinician may compare on-target responses and off-target effects across multiple configurations. In certain embodiments, a primary stimulation region exhibiting a field magnitude at least 10-20% greater than adjacent secondary regions (e.g., 1.2×A relative to a baseline field magnitude A) is identified as the optimal configuration when it consistently produces the desired physiological response with minimal off-target activation. Through this iterative adjustment process, the method enables identification of the directional stimulation field orientation that yields the maximum net therapeutic benefit prior to implantation of the indwelling electrode.

At step 410, the method proceeds with implantation of the indwelling electrode in the determined orientation after the optimal directional stimulation field has been identified. In various embodiments, a cylindrical segmented lead is advanced along the path of the surgical tool and positioned such that the primary stimulation region aligns with the anatomical target. In another embodiment, a self-expanding paddle electrode is deployed from within a cannula or sheath, unfolding into a preconfigured shape that provides a hemispherical stimulation field directed toward the target tissue. In certain embodiments, radiopaque markers, fiducial features, or orientation indicators on the electrode body or delivery tool may be used to confirm alignment of the primary stimulation region relative to the anatomy.

In certain embodiments, a self-expanding paddle electrode is compressed and rolled within the lumen of a cannula or introducer sheath during delivery. Upon deployment, the paddle expands into a pre-configured geometry that provides a hemispherical or otherwise directional stimulation field. To ensure that the expanded paddle is correctly aligned with the desired anatomical target, the system incorporates orientation and marker features that link the angular position of the cannula to the final orientation of the expanded electrode.

In one embodiment, the paddle electrode includes one or more marker features 26, such as radiopaque fiducials, surface notches, or patterned indicia, disposed along the electrode body or substrate. These markers are registered with corresponding features on the cannula, such as keyed slots, alignment tabs, or radiopaque bands, so that the relative angular orientation between the paddle and the cannula is fixed during delivery. As a result, when the physician rotates or positions the cannula to generate an optimal intraoperative directional stimulation field, the subsequent expansion of the paddle electrode will reproduce that same field orientation.

The integration of marker features provides significant procedural advantages. During intraoperative mapping, the cannula itself may be used to deliver a directional test field through apertures, surface electrodes, or a conductive stylet. Once the physician identifies the stimulation direction that yields the best therapeutic response, the cannula is held in that position. When the self-expanding paddle is advanced and released, its orientation relative to the cannula ensures that its active electrode surface is directed toward the intended target. Because the paddle expands into a known angular alignment dictated by the cannula and the markers, no further rotation, repositioning, or repeated testing is required after deployment.

In certain embodiments, the marker features may be visible under fluoroscopy, ultrasound, or other imaging modalities, allowing confirmation of electrode orientation relative to anatomical landmarks. In other embodiments, tactile or keyed coupling between the paddle and cannula ensures mechanical alignment even without imaging. In all cases, the cooperation of marker features on the paddle and cannula enables precise and reproducible delivery of a self-expanding electrode in the same orientation that was identified during mapping, thereby preserving therapeutic selectivity, improving surgical efficiency, and reducing the risk of off-target stimulation.

By securing the indwelling electrode in the identified orientation, the method ensures that long-term stimulation will consistently deliver energy into the primary stimulation region while minimizing activation of adjacent secondary regions. While the preferred embodiment uses electrical stimulation to excite the biological tissue, other stimulation methods including ultrasonic or optical means are envisioned.

In various embodiments, the directional neuromodulation system disclosed herein may incorporate features of self-expanding electrode leads, such as those described in connection with the electrophysiology lead system described in U.S. patent application Ser. No. 18/647,887, incorporated herein by reference in its entirety. In particular, because the present system delivers therapy electrodes through a temporary surgical tool (e.g., catheter, cannula, introducer sheath), the expandable lead architectures are well-suited for use in this minimally invasive context. The expandable lead may be inserted into the lumen of the mapping tool in a compressed configuration and, upon deployment, may self-expand to form a directional electrode surface oriented in the same direction identified during intraoperative mapping.

In some embodiments, the longitudinal body, winged substrates, and electrodes may be employed to achieve the benefits of directional field generation after catheter-based delivery. For example, the expandable substrate can increase the effective surface area contacting the target neural tissue, while the electrodes mounted on the substrate may be aligned to generate a preferred stimulation field. By combining the mapping workflow described herein with the expandable electrode features, clinicians may first identify the most effective stimulation orientation using a temporary mapping tool and then deploy a self-expanding directional lead in that same orientation, ensuring precise and consistent therapy.

In further embodiments, anchoring mechanisms, bending zones, urging layers, and geometric guides from the lead system may be applied to the current system to enhance deployment, reduce migration, and maintain orientation. For example, edge features or hole features integrated into the expandable substrate may resist longitudinal or rotational displacement after the lead is expanded within the anatomy. Geometric guides may facilitate smooth compression of the lead during insertion into the catheter and controlled re-expansion during deployment. These features are particularly advantageous when delivering electrodes through narrow percutaneous access routes, as they maintain structural reliability and directional consistency without requiring open surgical exposure.

Accordingly, the present application contemplates embodiments where the directional mapping tool and the self-expanding electrode lead are functionally integrated: the mapping tool identifies the optimal directional orientation, and the expandable lead is subsequently deployed in that same orientation, leveraging the prior system's compressible/expandable design to achieve stable, efficient, and minimally invasive directional neuromodulation therapy.

At step 412, after the indwelling electrode has been positioned and oriented to align the primary stimulation region with the desired anatomical target, the surgical tool is withdrawn from the patient while leaving the electrode implanted in situ. In some embodiments, the surgical tool may be temporarily removed and reintroduced for repositioning or confirmation of electrode orientation, but in the final stage the tool is fully removed to eliminate unnecessary hardware and reduce the risk of tissue irritation or infection. The indwelling electrode remains anchored within the biological tissue in the optimized position, maintaining directional coupling of stimulation through its primary stimulation region while insulating secondary regions. By removing the surgical tool and leaving only the therapeutic electrode in place, the method transitions from intraoperative testing and optimization to long-term therapy delivery.

At step 414, the implanted electrode is used to deliver long-term neuromodulation therapy through its primary stimulation region. In some embodiments, the electrode is connected to an implantable pulse generator that supplies electrical stimulation in accordance with programmed therapy parameters. In other embodiments, stimulation may be adjusted dynamically through physician programming, patient-controlled inputs, or closed-loop feedback algorithms that monitor physiological signals such as electromyography, evoked compound action potentials, respiratory airflow, or blood oxygenation. By maintaining stimulation through the primary region while minimizing activation of secondary regions, the indwelling electrode provides consistent and durable therapeutic benefit. Over time, the method enables optimization of therapy delivery for treatment of conditions such as obstructive sleep apnea, chronic pain, cardiovascular disorders, or pelvic dysfunction, while reducing energy consumption, improving selectivity, and limiting adverse off-target effects.

The process 400 then comes to an end.

It should be understood that in various embodiments the method uses one or more surgical tools (e.g. guidewire, introducer, needle, cannula, sheath, dilator, electrode) to aid in the delivery of a directional stimulation field and the indwelling electrode. In some embodiments, the surgical tool may include a guidewire, which is used to guide the indwelling electrode to the target along a desired path and assist with the delivery of a directional stimulation field. In some other embodiments, the surgical tool may include of a cannula which is used to pass inside an indwelling electrode to the desired target.

In various embodiments, the application of the directional stimulation field 10 is aided by the presence of the surgical tool 20. The surgical tool 20, may include for example, a canula or catheter. In one embodiment, a guidewire 24 may mechanically guide a directional electrode 14 (e.g., paddle or cylindrical) to a desired target and facilitate the application of a directional stimulation field 10. The guidewire 24 may also perform the stimulation or may rely on the indwelling electrode 14 to perform stimulation. In another embodiment, a cannula 20 is used to deliver an indwelling electrode 14 and facilitate the application of directional stimulation field 10. The cannula itself may contain stimulating electrodes for delivering a directional field, or the cannula may contain guiding features (e.g., apertures) which restrict the stimulation field to a particular orientation, direction, or region.

In another embodiment, an inner stimulating component (e.g., dilator core, indwelling electrode) may contain stimulating surfaces which provide a directional stimulation field (FIG. 6). The stimulation field may be provided directly through the catheter or cannula (e.g. stimulation through a mesh/perforated catheter) or through stimulation may be directed through apertures or other features in the catheter or cannula. In all cases, the surgical tool is used to facilitate the application of a directional stimulation field.

The method then identifies and determines a desirable directional stimulation field configuration for a net therapeutic benefit. For example, one directional stimulation field configuration may be applied and a physiological response to stimulation may be observed (e.g., airway opening, changes in heart-rate, reduction in pain, decreased inflammatory biomarkers). Desirable therapy may be evaluated using other physiological function biomarkers (e.g., motor recruitment, electromyography, sensory perception threshold feedback, heart rate change, inflammatory markers, evoked compound action potentials, etc.) Additional directional stimulation field configurations may be tested until a desired therapeutic benefit is observed. The stimulation field may be adjusted spatially to maximize the physiological therapy dose while minimizing any off-target and unwanted physiological responses (e.g. pain, heart rate, etc.). Once the desired benefit is determined, the desirable directional stimulation field may be used to inform the final positioning of the indwelling device (e.g. electrode lead). The indwelling device is then delivered to the tissue and used to apply a directional stimulation field resulting in the desired net therapeutic benefit.

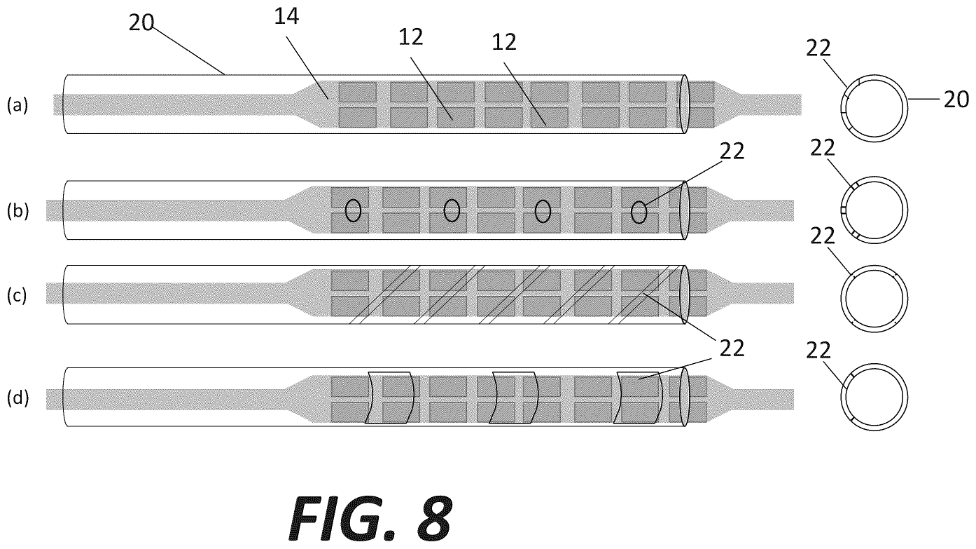

In some embodiments, the method employs a cylindrical segmented directional lead (FIG. 5A) using the surgical tool to apply a directional stimulation field. In a some other embodiments, a self-expanding paddle electrode (FIG. 5B) may be used to deliver a directional stimulation field. The self-expanding paddle may be passed directly along a guidewire or within a delivery cannula to the desired target. The self-expanding paddle lead 14 may unfold from a cannula to a pre-configured shape at the desired therapy site. Marker features on the lead (e.g. body, paddle, radiopaque) and surgical tool may enable the directional field orientation to be known with respect to one another or with respect to anatomical landmarks. (FIG. 9)

After the paddle is fully-expanded, it provides a wider treatment area (e.g., 3-4×wider than a delivery catheter tool diameter). A paddle also utilizes surface electrode contacts which provide directional (hemispherical) field coupling and energy efficiency benefits. Thus, the energy, directional field, and anti-migration benefits of a paddle lead is realized in illustrative embodiments using a self-expanding device, avoiding the need for surgery or general anesthesia to implant.

After the indwelling electrode is implanted and the desirable therapeutic benefit is confirmed, the surgical tool is removed. In other embodiments the tool may be temporarily removed and replaced, but the tool remains necessary for the delivery of a directional stimulation field. After the surgical tool is removed, the indwelling electrode is used to provide a directional stimulation field to achieve a net therapeutic benefit in the person.

To deliver a directional field using an indwelling implant for a net therapeutic benefit to a patient, multiple surgical tools and implanted lead features may be utilized.

The delivery of a directional stimulation field at the time of implant, in one embodiment may be accomplished through the use of a catheter introducer/cannula 20. In some embodiments, the catheter 20 introducer may incorporate stimulation electrodes that are temporarily used during the procedure. The catheter 20 sheath may contain one or more directional electrodes on the surface. These electrodes can be activated by a wired connection to a stimulator during the procedure. In another embodiment, one or more electrodes positioned radially on the catheter sheath may provide selective intraoperative stimulation in two or more directions, radiating from the center. The catheter 20 in conjunction with an introducer may also contain a handle that can be rotated to select the different electrodes and assist the user in quickly identifying the most effective direction of stimulation without rotating the entire catheter sheath. Further, the electrodes may also be positioned in staggered formation axially along the length. In some embodiments, each of the sheath's built-in directional electrodes are wired to a port that connect into a pulse generator that can multiplex and select the desired electrode as part of a control system.

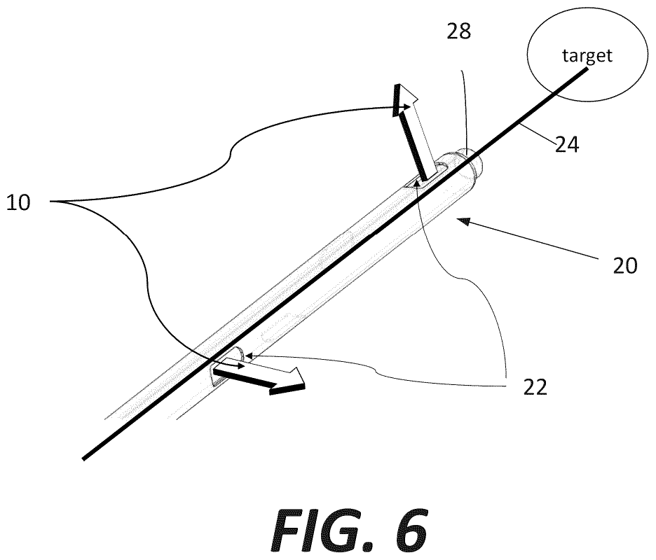

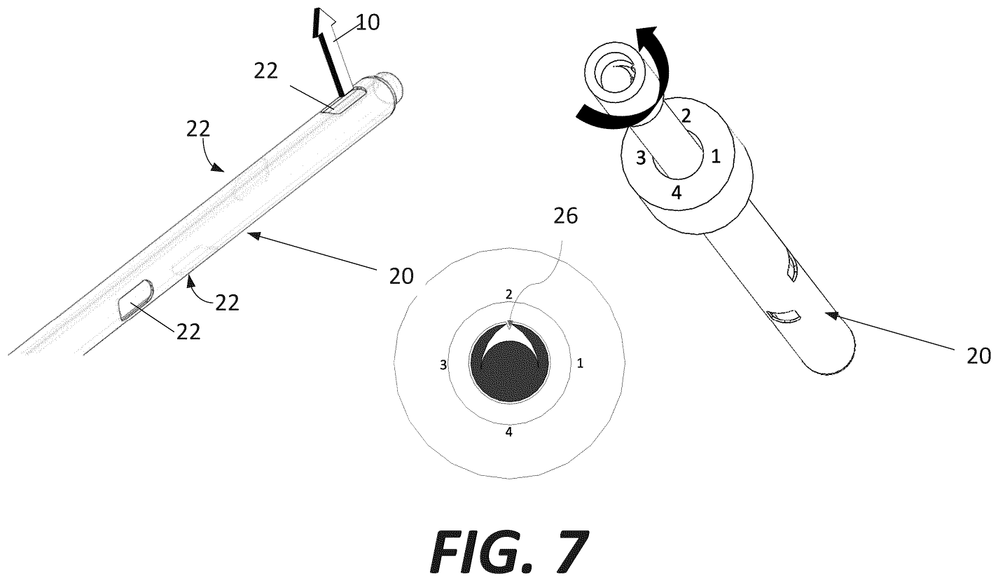

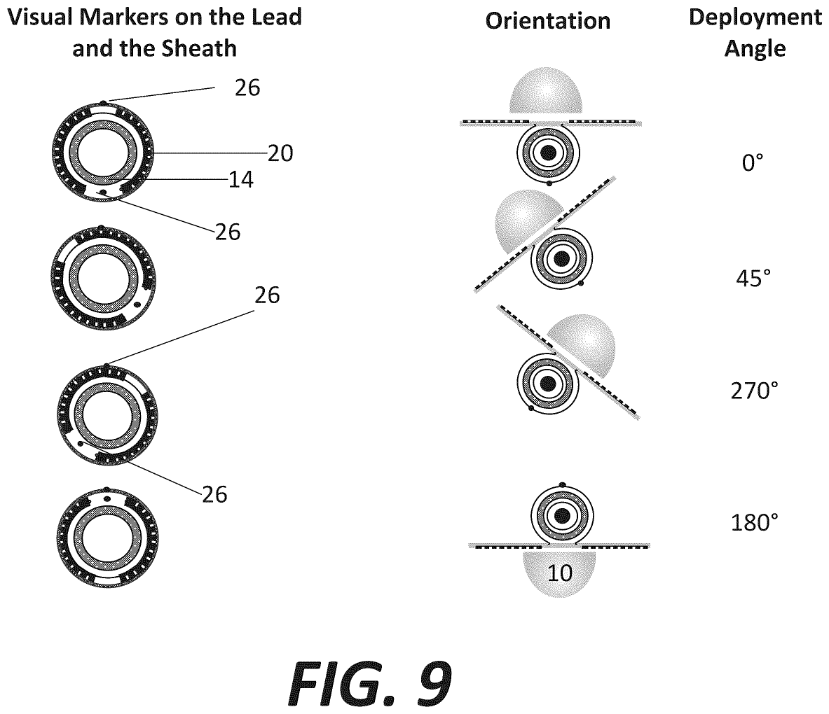

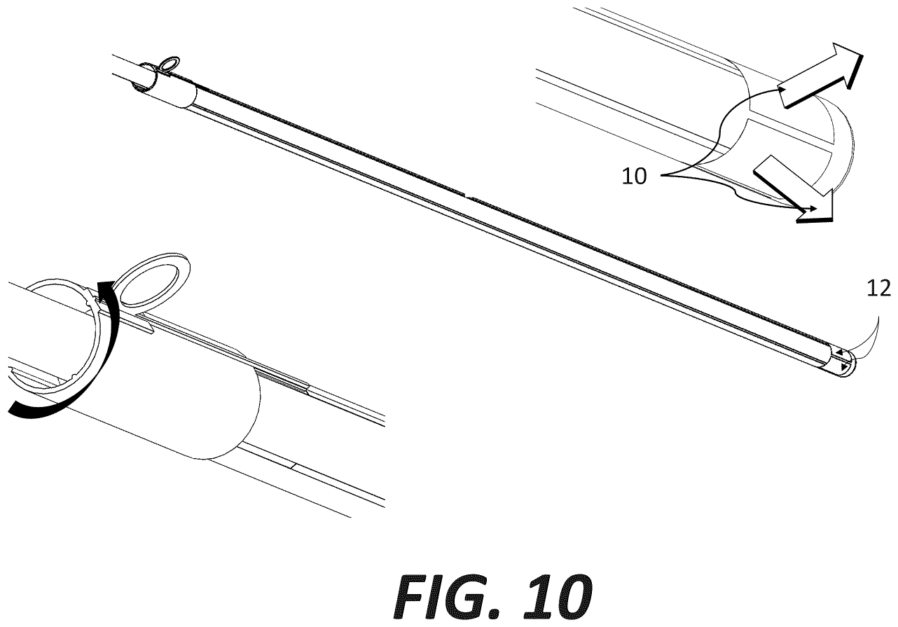

In another embodiment, the catheter 20 may work in conjunction with a stimulating element within the catheter to deliver a directional stimulation field. For example, the indwelling electrode (e.g., cylindrical or self-expanding paddle) within the catheter may serve as a stimulating source. In another embodiment, a conductive guidewire or conductive introducer may be applied within the catheter to serve as a stimulating source. (FIG. 10, FIG. 11). The catheter may also contain one or more aperture cutouts that exposes the one or more stimulating element surfaces within the catheter (FIG. 7). The apertures allow for the stimulation field to be directed through the apertures. The direction of the field may be changed by using multiple apertures with different orientations, or by rotating the aperture mechanically relative to the tissue. In another embodiment, the catheter may have a plurality of apertures that allow for simultaneous stimulation of two or more electrodes to generate bipolar local field stimulation rather than limitation of monopolar (FIG. 8). In some embodiments, the apertures may be staggered so that the various levels of the electrodes on the lead can be selected by rotating a handle on the proximal portion of the Introducer catheter. This may assist in the rigidity and structure of the sheath, rather than having multiple windows or large apertures that could lead to buckling during insertion. Staggered apertures direct mechanical strain into different radial planes providing superior column buckle load force.

FIG. 6 illustrates an embodiment of a directional introducer catheter 20 configured to both generate a directional stimulation field intraoperatively and to preserve orientation for deployment of a self-expanding paddle lead. The catheter 20 includes an elongate tubular body defining a central lumen sized to receive an implantable electrode lead. The catheter 20 further incorporates apertures 22 extending radially through the catheter wall, each aperture configured to direct stimulation energy toward a localized region of adjacent tissue. In the illustrated embodiment, one aperture 22 is positioned near the distal tip of the catheter while another aperture 22 is positioned along a more proximal region of the shaft, though it shall be appreciated that the longitudinal placement of apertures may vary or be aligned at the same axial position depending on the desired stimulation pattern.

During intraoperative use, the catheter 20 may contain or house a stimulating element, such as a conductive stylet or core 24, that generates a stimulation field. When energized, the stylet 24 emits current throughout its length; however, the apertures 22 restrict the emission path, causing the stimulation to be delivered directionally through the openings. In this manner, a directional stimulation field is produced despite energizing the entire stylet, allowing the physician to identify the optimal angular orientation for therapy. Alternative embodiments may include electrodes printed on the outer surface of the catheter 20, wires embedded in the wall of the catheter with exposed segments at defined angular locations, or combinations thereof. Each of these embodiments is capable of producing or permitting a directional field sufficient for intraoperative mapping.

In illustrative embodiments, the catheter used for directional mapping may incorporate a variety of stimulation features during the intraoperative phase. For example, the catheter may include printed electrodes, a flexible circuit, or wires integrated into the catheter wall. In other embodiments, a conductive stylet may be positioned down the center of the catheter lumen to act as a stimulation source. Still further, the catheter lumen itself may receive a stimulating element such as a permanent lead that is ultimately deployed, including but not limited to a rolled paddle electrode, a cylindrical ring-based lead, or a segmented directional electrode. In such embodiments, the permanent lead functions both as the mapping element during intraoperative stimulation and as the long-term therapy delivery device once the catheter is removed.

In various embodiments, the ability to use either integrated catheter electrodes or a permanent lead inserted within the catheter lumen ensures that the system is agnostic to the particular stimulation source. During directional mapping, the catheter assists in orienting the stimulation field and identifying the optimal angular direction for therapeutic effect. Once the correct orientation is found, the catheter is withdrawn, leaving the permanent electrode in situ in the identified orientation. This workflow allows clinicians to achieve precise directional targeting without having to rotate, reposition, or otherwise manipulate the permanent lead after deployment.

This approach provides a significant advantage over conventional systems that rely on omnidirectional cylindrical leads or require open surgical placement of paddle electrodes. The disclosed catheter facilitates directional stimulation without requiring repeated axial movement of the catheter or repositioning of the internal electrode to reach the target orientation. Instead, the mapping and therapeutic directionality are identified seamlessly in one process, and once optimized, the catheter is retracted, leaving the permanent electrode properly oriented for long-term therapy.

After a preferred stimulation direction is identified, the lead 14 (e.g., the self-expanding lead shown in FIGS. 31A-1B, 35C-35D) is advanced through the catheter lumen. Retraction of the catheter 20 relative to the lead permits the substrate 120 and wings 125 of the lead to expand outward into the extended configuration. Because the catheter 20 preserves orientation through fiducial markings, keyed handles, or radiopaque alignment features, the expanded paddle surface of the lead 14 deploys in the same angular orientation that was confirmed using the directional apertures 22. This ensures that the therapeutic configuration selected intraoperatively is faithfully maintained during chronic implantation without requiring subsequent repositioning or rotation of the lead.

In another embodiment, the catheter may have an introducer stylet that acts as a conductive metallic core 28 (FIG. 6). The metal stylet can act as the main conductor for stimulation (e.g. monopolar). The tip of the core may be exposed in tissue to deliver local monopolar stimulation. The introducer sheath may also have narrow partial circumferential windows cut along it its length. These windows expose the metal core 28 to deliver directional stimulation in a direction normal to the catheter. In some embodiments, the stylet may be partially coated with a dielectric (e.g. PTFE) such that as the stylet is rotated independently inside of the introducer, a non-coated portion of the stylet lines up with one of the introducer's window to deliver selective stimulation through a chosen window. For instance, this embodiment allows a user to discriminately select between omnidirectional introducer tip stimulation and directional stimulation via introducer window that has no overlap between the two. In another embodiment, the stylet may have a segment of its surface that is isolated and provides a separate electric conductor to deliver directional stimulation that enables local bipolar stimulation between the tip and the window.

In various embodiments, the catheter includes both a guidewire 24 for positioning and advancement, as well as a conductive metal core blunt tip 28 that protrudes slightly beyond the distal end of the catheter. The guidewire may be used in a conventional manner to navigate the catheter to the target anatomical location, while the blunt conductive tip serves as a stimulating electrode capable of delivering test stimulation in a directional manner.

The inclusion of the blunt conductive tip 28 enables the catheter itself to serve a dual role: first as a navigational instrument, and second as a diagnostic electrode for field testing. The exposed metal core tip may be energized to apply a directional field to adjacent neural structures, allowing the physician to assess physiological responses prior to permanent electrode placement. Once an optimal directional orientation is determined, the catheter 20 can be used as a delivery channel for advancing a permanent therapy electrode along the same trajectory, while the temporary blunt tip and guidewire are retracted.

This dual-element configuration provides increased precision during implantation by combining structural guidance with immediate feedback on electrode orientation. In certain embodiments, the conductive tip may be radiopaque or otherwise marked to facilitate visualization and alignment under fluoroscopy or other imaging modalities.

FIG. 6 further illustrates embodiments of the catheter 20 designed for intraoperative mapping and subsequent delivery of a permanent therapy electrode. In some embodiments, the catheter 20 is configured to receive a rigid conductive core 52 that provides both mechanical rigidity and directional stimulation capability. Without the core 52, the catheter 20 would lack the structural stiffness necessary for advancement through soft tissue, functioning much like a flexible straw. With the core 52 inserted, however, the catheter 20 can be safely advanced toward the neural target while maintaining directional control. The core 52 may terminate in a semi-blunt conductive tip 54, which facilitates advancement through soft tissue planes without sharp cutting or risk of perforation.

During mapping, the catheter 20, in combination with the conductive core 52, is capable of generating a directional stimulation field through one or more apertures 56. This allows the physician to identify an optimal orientation for therapeutic stimulation by observing physiological responses such as electromyography (EMG) activity, muscle recruitment, or changes in airway patency. Once the preferred orientation has been identified, the conductive core 52 may be withdrawn, leaving the catheter 20 in situ as a delivery conduit. A permanent therapy electrode 58, such as a self-expanding paddle, segmented cylindrical lead, or other directional electrode, may then be advanced through the lumen of the catheter 20 and deployed in the same orientation identified during the mapping process. After electrode placement, the catheter 20 is withdrawn, leaving the therapy electrode adjacent the neural target.

This workflow provides a significant advantage over conventional approaches, where a physician would be required to reposition, rotate, or otherwise manipulate a stiff introducer or electrode to approximate the previously determined “best” orientation. Rotational manipulation of rigid introducers often results in undesirable displacement, such as movement from one neural branch to another. In contrast, the catheter 20 of FIG. 6 directly assists in both mapping and orientation alignment, thereby eliminating the need for repeated direction finding or complex manipulations once the target direction has been identified. In practice, the procedure reduces to a streamlined sequence: (1) insertion with directional mapping, (2) withdrawal of the core, (3) delivery of the permanent therapy electrode, and (4) removal of the catheter. The physician thus performs a single orientation-finding step, after which the permanent therapy electrode is deployed in the desired alignment without additional testing or adjustment.

FIG. 7 illustrates the directional catheter system 20 used to both identify an optimal stimulation direction and deliver a permanent therapy electrode in that same orientation. In operation, the catheter 20 is first introduced into the patient proximate to a target neural structure. The catheter 20 incorporates directional stimulation features, such as apertures, surface electrodes, or an internal conductive stylet, as described above with respect to FIG. 6. These features allow the catheter 20 to generate or assist in generating a directional stimulation field. During intraoperative use, the physician may apply stimulation through the catheter 20 and observe physiological responses such as electromyography (EMG), muscle activation, or airway patency, thereby identifying the orientation that produces a desirable therapeutic effect.

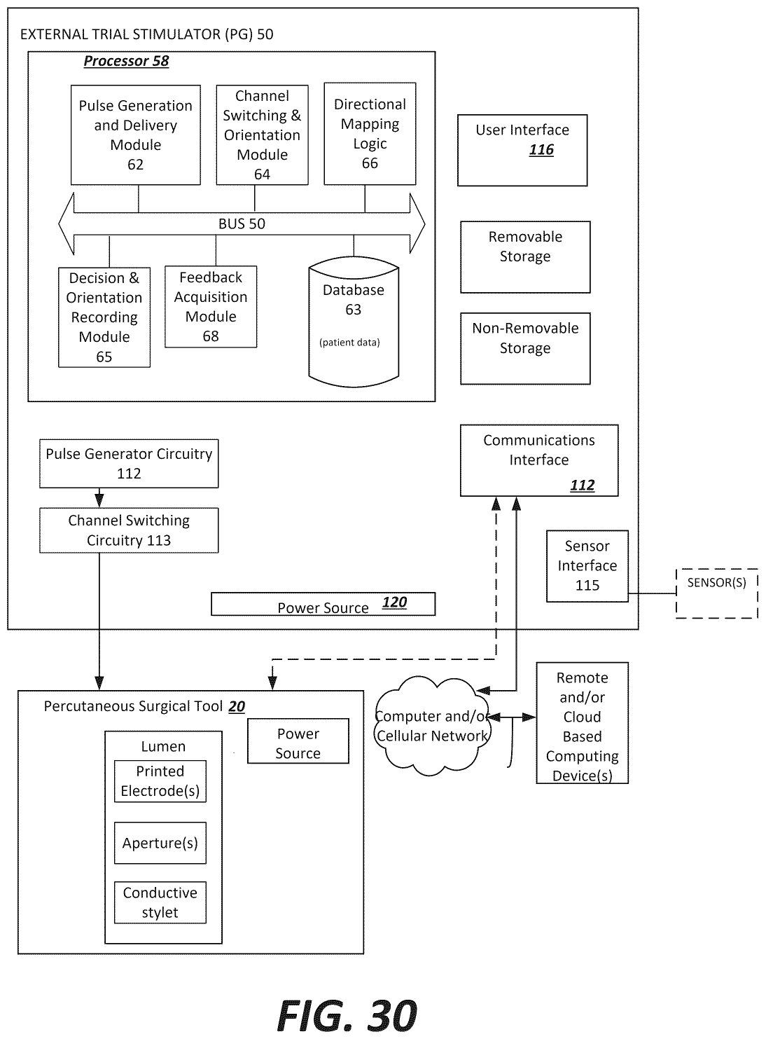

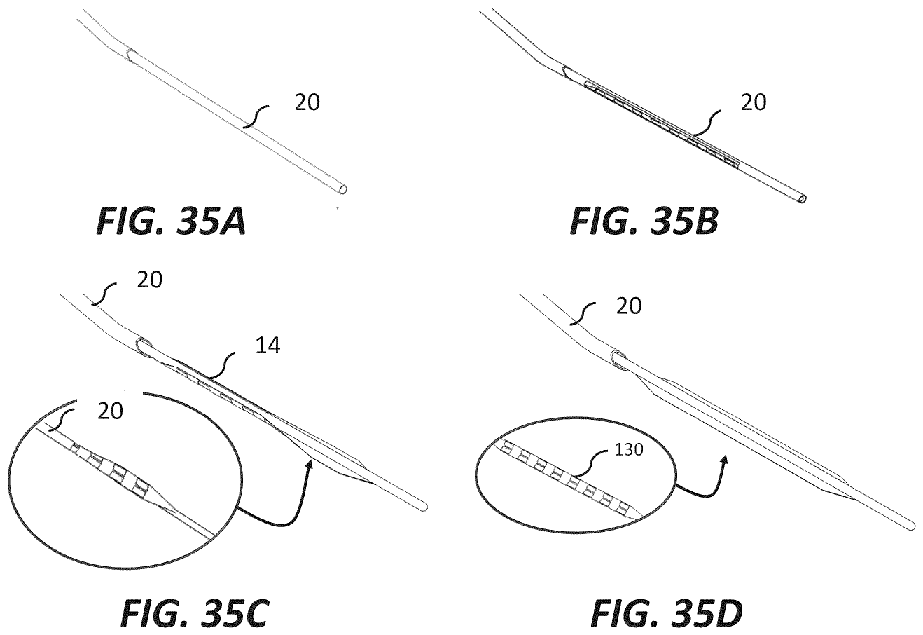

After the optimal orientation is identified, a therapy electrode 14 is advanced through the lumen of the catheter 20 and positioned at the treatment site. The therapy electrode may be, for example, a segmented cylindrical lead or a self-expanding paddle lead as described in FIGS. 31A-31B and 35C-35D. The catheter 20 thereby serves as a surgical tool that establishes orientation and as a delivery conduit for the permanent implant. After the therapy electrode 14 is positioned in the selected orientation, the catheter 20 is retracted and removed, leaving the therapy electrode in place.

A particular advantage of the embodiment shown in FIG. 7 is that the therapy electrode 14 is automatically deployed in the same orientation as was identified by the directional catheter 20, without requiring further manipulation or rotation. By integrating mapping and delivery functions into a single tool, the system ensures that the therapeutic configuration determined during mapping is preserved during chronic implantation. This reduces procedural complexity and eliminates the need for iterative repositioning or surgical manipulation that is often required with conventional directional electrodes.

In this way, FIG. 7 demonstrates a workflow in which the catheter 20 “points the way” by identifying a preferred stimulation direction, the therapy electrode 14 is deployed in that same direction, and the catheter 20 is withdrawn, leaving the electrode in its intended position. This seamless handoff between the mapping tool and the permanent implant ensures that the entire system “works in concert,” simplifying the procedure for the physician and improving reliability of therapeutic outcomes.

In various embodiments embodiment, the catheter 20 may be temporarily filled with a solid conductive core 52 that terminates in a semi-blunt tip 54. The semi-blunt tip 54 is not sharpened like a cutting needle, but instead is shaped to allow the catheter 20 to be advanced through soft tissue in a controlled manner without piercing or cutting. This arrangement allows the catheter 20 to serve as both a mapping introducer and a placement guide.

During the procedure, the conductive core 52 provides directional stimulation through one or more apertures or electrode windows 56 of the catheter 20. The physician may use this arrangement to map directional fields, observe patient responses, and identify the optimal stimulation orientation. Once the preferred orientation has been determined, the conductive core 52 can be withdrawn, leaving the lumen of the catheter 20 available to receive a permanent therapy electrode 58. The permanent electrode 58 is delivered through the bore of the catheter 20 and deployed in substantially the same orientation as identified during mapping. The catheter 20 is then removed, leaving the permanent electrode 58 in place.

By employing the semi-blunt tip 54, the catheter 20 can be advanced safely into target tissue regions while minimizing the risk of perforation. The combination of directional mapping capability and introducer-like structural features ensures that the mapping and therapeutic delivery steps occur in sequence and in alignment, thereby eliminating the need for subsequent manipulation or rotation of the permanent electrode once deployed.

FIG. 8 illustrates various embodiments of aperture configurations formed along the wall of a directional catheter 20 to permit delivery of a stimulation field through the catheter body. These apertures expose an underlying stimulating element, such as a conductive stylet, internal electrode, or indwelling leadso that electrical energy can be directed outward toward surrounding tissue.

In the embodiment of FIG. 8A, the catheter 20 includes a series of rectangular apertures 22 aligned axially along the shaft. Each aperture 30A provides a localized opening through which stimulation energy can be delivered. Selective activation of individual apertures may provide a directional mapping function in which different angular orientations are sequentially energized to identify a preferred therapeutic direction.

In the embodiment of FIG. 8B, the catheter 20 incorporates a series of circular apertures 22 spaced axially along the shaft. Circular apertures may simplify manufacturing, reduce stress concentration points, and provide uniform emission windows while still enabling directionally targeted stimulation.

In the embodiment of FIG. 8C, the catheter 20 features oblique slits 22 oriented at an angle relative to the longitudinal axis of the shaft. Such slits provide elongated emission regions that can direct stimulation fields at a broader angle, potentially covering multiple adjacent neural structures while maintaining directional selectivity.

In the embodiment of FIG. 8D, the catheter 20 includes curved apertures 22 extending across the catheter wall. Curved apertures may conform to the geometry of the underlying stimulating element and provide a greater circumferential coverage, thereby allowing for complex stimulation field shaping when combined with adjacent apertures.

It should be appreciated that the aperture geometry is not limited to rectangular, circular, oblique, or curved shapes as illustrated. Other forms, such as polygonal, staggered, or irregular apertures, may also be employed depending on desired electrical, mechanical, or manufacturing characteristics. The essential feature of these embodiments is that the apertures permit directional stimulation through the catheter wall, enabling intraoperative mapping of optimal stimulation orientations prior to deployment of a permanent therapy electrode.

FIG. 9 illustrates embodiments of a marker alignment system configured to ensure consistent orientation between a temporary directional catheter 20 and a permanent therapy electrode 14, such as a self-expanding paddle lead. As described previously, the catheter 20 is first used intraoperatively to generate or assist in generating a directional stimulation field, enabling the physician to determine an optimal angular orientation relative to the target neural structure. To preserve this orientation during implantation of the permanent therapy electrode 14, the catheter 20 and the electrode 14 are provided with corresponding alignment markers 26.

In the illustrated embodiment, the catheter 20 includes catheter markers 26 positioned circumferentially along its outer surface, while the therapy electrode 14 includes lead markers 26 positioned along the longitudinal body or substrate of the electrode. By aligning the catheter markers 26 with the lead markers 26 prior to advancement of the electrode through the catheter lumen, the system ensures that the permanent electrode deploys in the same orientation that was identified as therapeutically desirable during the mapping stage.

The markers 26 may take a variety of forms, including visual markings, radiopaque markers visible under fluoroscopy, echogenic markers visible under ultrasound, or mechanical alignment features such as keyed protrusions or grooves. The markers may be placed at the proximal end of the catheter 20 to assist the user in aligning components outside the patient's body, or may be placed at distal or intermediate regions for confirmation of orientation during imaging. In some embodiments, the therapy electrode 14 itself may serve as a marker by incorporating radiopaque or echogenic materials in its electrode 14 surfaces or along its substrate edges.

By providing corresponding marker systems, FIG. 9 ensures that once an optimal directional field is identified by the catheter 20, the permanent therapy electrode 14 can be delivered in that same angular orientation without requiring rotation, repositioning, or surgical manipulation. This seamless alignment workflow allows the physician to “map north” with the catheter, then “deploy north” with the permanent lead, thereby simplifying the procedure and reducing opportunities for error or off-target stimulation.

FIG. 10 illustrates the directional catheter system 20 having a series of directional electrodes 12 that are arranged in a linear formation along the catheter shaft. Each electrode 12 is configured to deliver stimulation energy outward from the catheter wall in a specific angular direction relative to the catheter axis.

In operation, the catheter 20 may selectively activate one or more of the electrodes 32 through connection to an external pulse generator or multiplexing controller. By energizing a particular electrode in the series, a stimulation field is generated in the corresponding angular direction (see arrows, FIG. 10). In this manner, the system permits directional steering of the stimulation field without requiring rotation of the catheter itself. For example, the physician may activate a first electrode to stimulate in one direction, deactivate the first electrode, and then activate a second electrode positioned at a different circumferential location to stimulate in another direction.

This embodiment allows rapid intraoperative mapping of stimulation directions by toggling between electrodes rather than mechanically repositioning the catheter. After a preferred stimulation direction is identified, the catheter 20 serves as a reference orientation guide for the delivery of a permanent therapy electrode 14, such as a segmented cylindrical lead or self-expanding paddle. As with the embodiments of FIGS. 6-9, the catheter may incorporate alignment markers to ensure that the permanent therapy electrode is deployed in the same angular orientation as was determined to be therapeutically beneficial during mapping.

By integrating multiple directional electrodes into the catheter wall and enabling selective activation, the embodiment of FIG. 10 provides a practical and efficient workflow for intraoperative mapping and therapy alignment. This configuration also reflects certain commercial implementations in which directional electrode arrays are printed, embedded, or otherwise integrated into the catheter sheath to provide robust and repeatable directional stimulation fields.

FIG. 11 illustrates various embodiments of the catheter configured to provide directional stimulation through apertures in its wall. The dilator 40 may contain one or more electrodes used to deliver a directional stimulation field. In these embodiments, the catheter includes one or more stimulating elements, such as printed electrodes disposed on the catheter surface or a conductive core disposed within the catheter lumen. The catheter further includes apertures 22 formed in its wall, through which electrical energy from the stimulating element is delivered. In this manner, the catheter itself provides a directional stimulation field during intraoperative mapping.

As shown in FIG. 11, the catheter 20 includes a series of axially spaced apertures 22 aligned along one side of its shaft. When the internal conductive core 28 is energized, current exits through the apertures 22, producing stimulation fields directed radially outward from the catheter. By mechanically rotating the catheter about its longitudinal axis, the apertures 22 may be oriented toward different anatomical regions, enabling the physician to steer the stimulation field without repositioning the distal tip of the catheter.

The catheter may include paired apertures 22 disposed circumferentially or axially relative to one another. Stimulation may be delivered between a pair of electrodes associated with different rows of apertures, producing a shaped field extending between the paired apertures. Such configurations allow the catheter to generate a localized field that targets a specific neural structure. Multiple pairs of apertures may be distributed around the catheter's 20 circumference, allowing selective steering of the therapeutic field by activating different aperture pairs.

In various embodiments, the catheter integrates both stimulating elements and directional apertures, enabling the same device to deliver a directional stimulation field for intraoperative mapping. Once the optimal angular orientation is identified using the catheter, a permanent therapy electrode, such as a segmented cylindrical lead or a self-expanding paddle lead, may be advanced through the catheter lumen and deployed in the identified orientation after the catheter is withdrawn.

In various embodiments, the catheter 20 may configured for a rotational mapping method. In this approach, the catheter 20 includes a plurality of apertures 22 arranged about its circumference, each aperture 70 being in communication with a conductive core 28 or electrode structure within the catheter. When electrical energy is applied, the stimulation field preferentially exits through the exposed aperture 22, thereby creating a directional field that may be rotated about the longitudinal axis of the catheter 20. By selectively energizing one or more apertures, or by physically rotating the catheter 20 so that a particular aperture is directed toward the target neural structure, the physician may sequentially test multiple orientations.

In use, the catheter 20 is first advanced toward the neural target, optionally over a conductive blunt core as described in connection with FIG. 6. A directional field is then generated through one or more apertures 70, and the physician observes the resulting physiological response, such as electromyography (EMG) activity, muscle contraction, or airway patency. The catheter 20 may be rotated incrementally until an orientation is achieved that maximizes therapeutic benefit while minimizing off-target effects. Once the optimal orientation has been identified, the conductive core 72 is removed, and a permanent therapy electrode, such as a segmented cylindrical lead or self-expanding paddle, is delivered through the catheter 20 lumen. The permanent electrode is thereby aligned in the same orientation that was identified during the rotational mapping step. The catheter 20 is then withdrawn, leaving the therapy electrode in place.

This rotational method may be considered in contrast with other embodiments (e.g., FIGS. 8-10) in which the catheter 20 inherently provides directional fields without the need for rotation. In both approaches, however, the catheter 20 directly assists in intraoperative directional mapping, preserves the identified orientation, and ensures that the permanent electrode is deployed in the desired direction without requiring repeated or redundant testing.

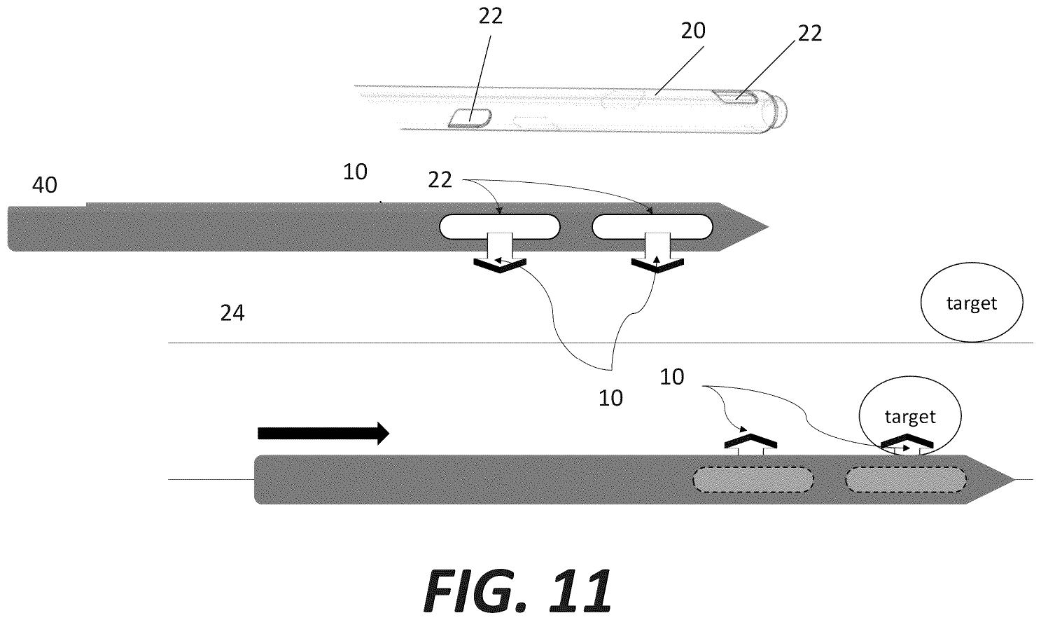

FIG. 12 illustrates a flowchart of a method 1200 for delivering neuromodulation therapy using the catheter to identify and preserve an optimal directional stimulation field. It should be noted that this method is substantially simplified from a longer process that may normally be used. Accordingly, the method shown in FIG. 12 may have many other steps that those skilled in the art likely would use. In addition, some of the steps may be performed in a different order than that shown, or at the same time. Furthermore, some of these steps may be optional in some embodiments. Accordingly, the process 1200 is merely exemplary of one process in accordance with illustrative embodiments of the invention. Those skilled in the art therefore can modify the process as appropriate.

The method begins with operation 1202, in which the catheter is inserted into the patient proximate to a target neural structure. In operation 1204, the catheter is advanced to a desired anatomical location, where it is configured to apply one or more directional stimulation fields through its integrated electrodes, apertures, or conductive stylet.

In operation 1206, the physician applies a directional field through the catheter and, at step 1208, observes a physiological response such as electromyography (EMG), muscle activation, airway patency, or other neural or muscular feedback. In operation 1210, the orientation of the directional field may be changed, for example by activating a different electrode, exposing a different aperture, or rotating the catheter. The physician then compares the physiological responses across orientations to determine which field provides the best therapeutic benefit relative to off-target effects.

In operation 1212, the physician selects the optimal directional field for therapy based on the comparative responses. In practice, this typically involves testing multiple directional fields to ensure that the chosen orientation is optimal, although in some cases the desired response may be observed on the first attempt. In operation 1214, the catheter, which has served as a temporary mapping tool, is removed while maintaining the identified orientation.

Finally, in operation 1216, a permanent therapy electrode, such as a segmented cylindrical lead or a self-expanding paddle lead, is delivered in the orientation corresponding to the selected directional field. The permanent electrode is then used to provide long-term stimulation therapy in the mapped orientation without requiring further manipulation or repositioning.

The method of FIG. 12 highlights the procedural workflow enabled by the catheter: (1) insertion, (2) directional field generation, (3) physiological response observation, (4) orientation adjustment and comparison, (5) selection of an optimal directional field, and (6) delivery of therapy using that orientation. By combining mapping and delivery into a single system, the method simplifies the surgical procedure and ensures that the therapeutic benefits identified intraoperatively are preserved during chronic therapy.

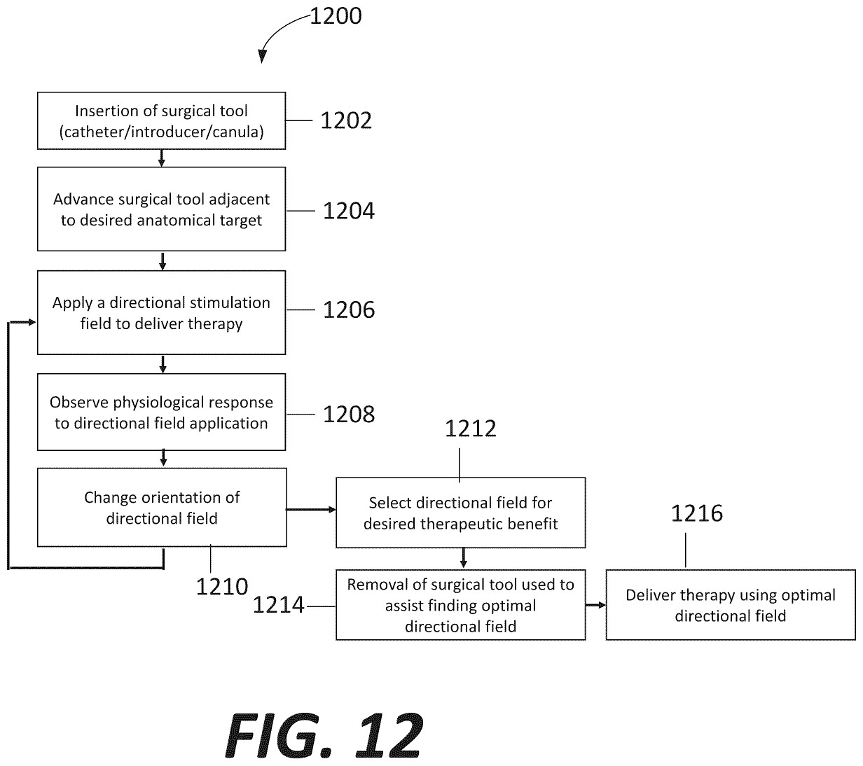

FIG. 13 illustrates an embodiment of a stimulation and feedback control system 1300 configured to operate the catheter and utilize physiological feedback to optimize delivery of a directional stimulation field. The system includes a stimulation and feedback controller 1310 having a processor 1312, stimulation generator 1314, and multi-channel stimulation output 1316. The controller is configured to deliver stimulation to the catheter, which in turn applies energy to stimulating electrodes with directionality positioned adjacent to neural tissue.

In operation, stimulation is applied via the catheter 20 in one or more angular orientations. The resulting physiological responses are monitored through a plurality of feedback sensors, such as electromyography (EMG) sensors 1330, oxygen saturation (SpO₂) sensors 1340, or other sensors placed on or near relevant anatomy (e.g., chest wall, finger, or oral airway). These signals are routed into input modules 1350 and processed by the controller 1310. The processor 1312 compares responses across different directional fields to determine which electrode orientation produces the most beneficial therapeutic effect, such as airway patency or muscle activation, while minimizing off-target responses such as airway collapse or unwanted muscle contraction.

The controller may also drive a display 1360 to provide real-time visualization of stimulation parameters and physiological feedback, enabling the physician to select or confirm the optimal directional field intraoperatively. A stimulation trigger 1370 may synchronize stimulation delivery with physiological events (e.g., respiration cycles) to further refine therapy.

As an illustrative example, in an upper-airway stimulation application, EMG signals may be collected from a muscle that collapses the airway and from a muscle that opens the airway. By comparing responses across directional fields generated by the catheter, the controller may determine which orientation preferentially activates airway-opening musculature while minimizing activation of airway-collapsing musculature. The selected orientation is then used to guide permanent electrode deployment, ensuring that long-term therapy replicates the optimal mapping outcome.

Thus, the embodiment of FIG. 13 demonstrates how the catheter may be integrated with a feedback-based control system to provide closed-loop optimization of directional stimulation fields, combining intraoperative mapping, physiological monitoring, and long-term therapy alignment into a single streamlined workflow.

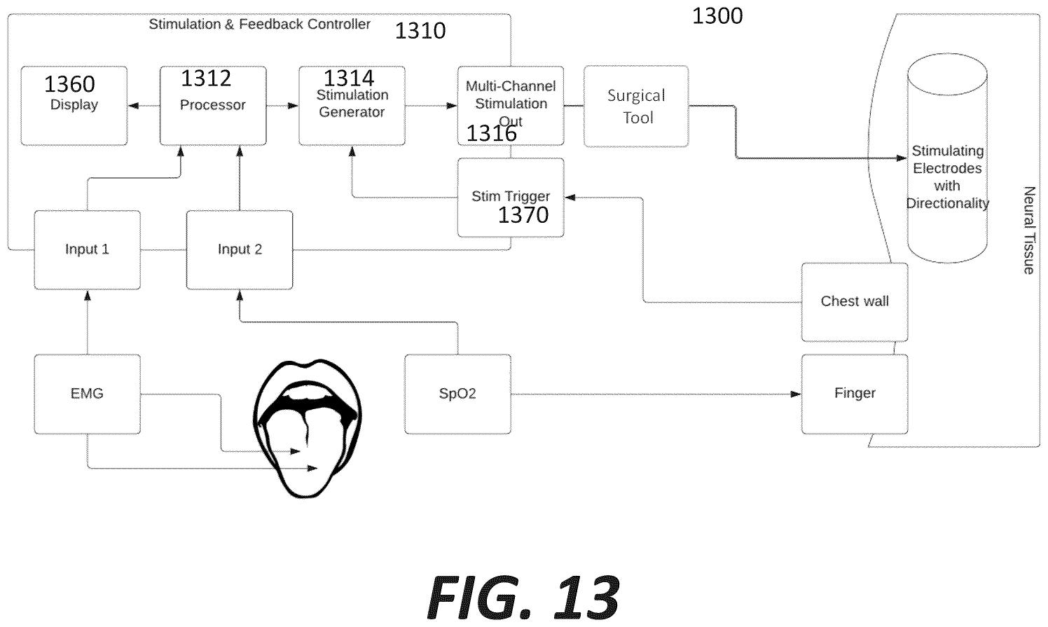

FIG. 14 illustrates an embodiment demonstrating the importance of orientation when using the catheter to deliver a directional stimulation field. In this example, the catheter 20 is positioned such that its directional field B is oriented improperly relative to the target neural structure. As a result, stimulation produces both on-target activation of a desired muscle group and off-target activation of an undesired muscle group.

Electromyography (EMG) signals are recorded from multiple anatomical sites, such as muscles of the tongue and upper airway. The EMG traces shown at the right demonstrate that both an on-target EMG response (indicating beneficial activation) and an off-target EMG response (indicating adverse activation) are present simultaneously. Although each signal individually may appear therapeutically valid, their combination results in summation of conflicting effects, which reduces the net therapeutic benefit and may even exacerbate the clinical condition (e.g., airway collapse instead of airway opening).

The catheter 20 advantageously enables the ability to map and compare multiple directional fields prior to permanent electrode implantation. By testing stimulation in different orientations and recording corresponding EMG responses, the physician may distinguish between orientations that produce pure on-target activation versus those that yield a mixture of on-target and off-target effects. The catheter thereby enables intraoperative identification of the optimal orientation, e.g., one in which the on-target EMG response is maximized and off-target responses are minimized or eliminated.

Following such mapping, the permanent therapy electrode may be deployed through the catheter in the identified optimal orientation, ensuring that the final implant provides therapeutic benefit without the undesirable summation effects illustrated in FIG. 14.

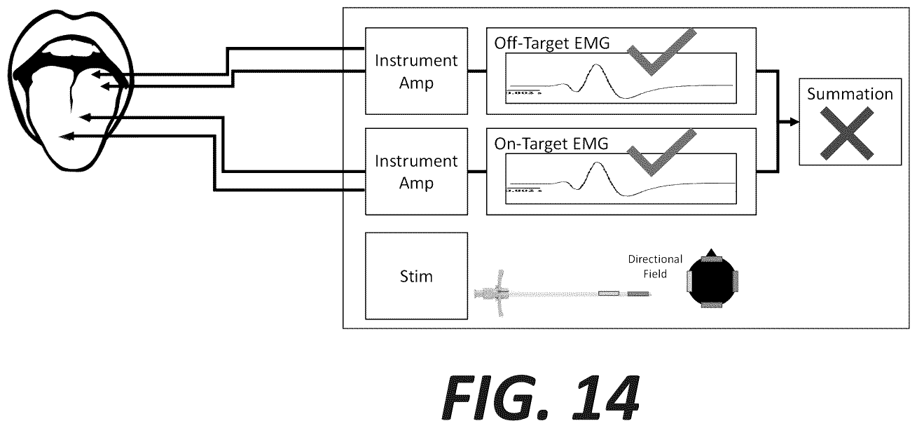

FIG. 15 illustrates an embodiment in which the catheter 20 is oriented to produce a purely on-target directional field, yielding the desired therapeutic effect. In this case, the stimulation field is directed toward the intended neural structure, while avoiding adjacent off-target tissue. As a result, the recorded on-target EMG response shows strong, consistent activation, whereas the off-target EMG recording shows no measurable response.

This selective activation ensures that the therapeutic effect (e.g., opening of the upper airway through activation of a target tongue muscle) is achieved without simultaneously producing adverse or counterproductive effects, such as activation of muscles that contribute to airway collapse. The absence of off-target EMG signals confirms the precision of the catheter's directional mapping capability.

By comparing the results of FIGS. 14 and 15, it can be seen that the surgical tool (e.g., the catheter 20) allows clinicians to distinguish between orientations that produce mixed or conflicting responses and orientations that produce selective, beneficial activation. Once the optimal orientation is identified intraoperatively using the catheter, the permanent therapy electrode may be deployed in the same orientation to provide consistent therapeutic benefit during chronic use.

FIGS. 14 and 15 together illustrate the importance of intraoperative directional mapping using the catheter 20. As shown in FIG. 14, when the catheter is oriented improperly, stimulation produces both on-target EMG activity and off-target EMG activity. This combination leads to a summation of conflicting effects, reducing the net therapeutic benefit and in some cases introducing adverse outcomes.

In contrast, FIG. 15 demonstrates the catheter oriented in the optimal direction. Here, stimulation produces a robust on-target EMG response while eliminating any detectable off-target activity. The absence of off-target responses confirms that the directional field is confined to the desired neural structure, providing a clear and unambiguous therapeutic effect.

Together, these figures illustrate the workflow enabled by the catheter: clinicians may test multiple orientations, compare the physiological responses, and identify the configuration that maximizes therapeutic efficacy while minimizing side effects. Once the optimal orientation is established, a permanent therapy electrode may be delivered through the catheter and deployed in the same orientation, thereby ensuring that the beneficial mapping result is preserved during long-term therapy.

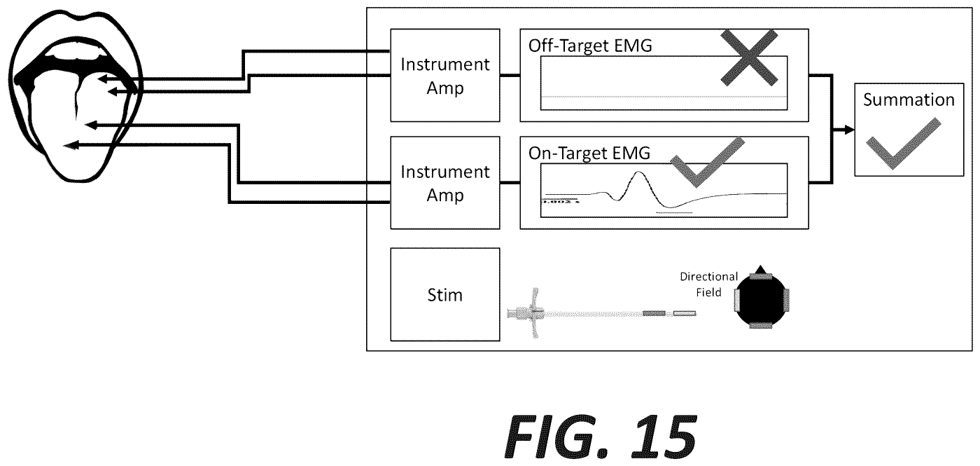

FIG. 16 illustrates a process of a method for deploying a self-expanding directional electrode following intraoperative mapping with the catheter 20. It should be noted that this method is substantially simplified from a longer process that may normally be used. Accordingly, the method shown in FIG. 16 may have many other steps that those skilled in the art likely would use. In addition, some of the steps may be performed in a different order than that shown, or at the same time. Furthermore, some of these steps may be optional in some embodiments. Accordingly, the process is merely exemplary of one process in accordance with illustrative embodiments of the invention. Those skilled in the art therefore can modify the process as appropriate.

The process may begin by inserting the catheter 20 into the patient and applying one or more directional stimulation fields 10 as described above. The stimulation fields are sequentially activated and their corresponding physiological responses are observed, allowing the physician to identify an optimal stimulation orientation relative to the target neural structure.

After the optimal orientation is identified, the method proceeds by aligning a self-expanding paddle electrode 14 with the orientation established by the catheter 20. This alignment may be accomplished in a variety of ways, including but not limited to: fiducial markers on both the catheter and the paddle lead, keyed coupling features that mechanically constrain the orientation, or orientation-maintaining engagement features between the catheter lumen and the lead. In this way, the self-expanding paddle is deployed in the same angular orientation that was determined to be therapeutically optimal during the mapping procedure.

Following alignment, the self-expanding paddle lead is advanced through the lumen of the catheter and deployed at the target location. Upon deployment, the paddle expands to its extended configuration, thereby presenting its electrodes in the identified orientation. The catheter is then withdrawn, leaving the paddle in place to deliver long-term directional stimulation therapy.

This method provides the advantage of preserving the mapping result without requiring additional manipulation, rotation, or repositioning of the permanent electrode. By ensuring that the self-expanding paddle opens in the same direction that produced the optimal intraoperative response, the therapy electrode is immediately effective, reduces procedure time, and minimizes the risk of off-target stimulation.

In some embodiments of the method illustrated in FIG. 16, additional features are provided to ensure that the self-expanding paddle electrode maintains the mapped orientation as it is deployed from the catheter 20. Because the paddle transitions from a compressed configuration inside the catheter to an expanded configuration at the target tissue, it is desirable to confirm that the orientation identified during mapping is preserved after deployment.

To this end, the paddle may include orientation-preserving features that prevent rotation as it is advanced through the catheter. For example, the paddle may be keyed to the catheter lumen such that it cannot rotate relative to the catheter while being delivered. In other embodiments, radiopaque markers or other fiducials are disposed on both the catheter and the paddle electrode to allow fluoroscopic confirmation that the deployed paddle has expanded in the intended orientation.

Additionally, the correct orientation may be confirmed functionally by applying stimulation after deployment and comparing the observed physiological response to that identified during mapping. For example, once the paddle has expanded, the physician may briefly stimulate through the paddle's electrodes to confirm that the desired on-target activation is achieved without off-target activation.

These features provide confidence that the self-expanding paddle electrode remains aligned with the optimal stimulation direction determined during mapping, thereby reducing the risk of orientation drift during delivery and ensuring that the therapeutic benefit identified intraoperatively is preserved in chronic use.

In some embodiments, the size of the paddle electrode itself may be varied to achieve a desired balance between coverage and precision. A relatively large paddle may provide increased surface area and stability, but may also increase the likelihood of off-target activation if placed near multiple neural structures. Conversely, a smaller paddle may provide more selective activation at the expense of reduced anchoring surface or coverage area.

Intraoperatively, selectivity is often optimized through electrode programming, for example, by activating different combinations of electrodes on a single paddle lead to direct the stimulation field with greater precision. However, in certain cases where electrode programming alone cannot sufficiently limit off-target responses, alternative paddle geometries or sizes may be employed. This allows a physician to select between multiple paddle models depending on the patient's anatomy and therapeutic goals.

In various embodiments, the disclosed system is configured to maximize on-target therapeutic effects while minimizing off-target responses. For example, in the case of hypoglossal nerve (HGN) stimulation for obstructive sleep apnea, it may be desirable to activate tongue protrusor muscles that open the airway while avoiding activation of retrusor muscles that would undesirably close the airway. In other embodiments directed to pain therapy, the system may be configured to stimulate a nerve trunk or branch associated with analgesia while avoiding stimulation of adjacent muscle fibers that could cause discomfort or involuntary contraction. Off-target effects may be minimized by maintaining stimulation at or below a sub-perception or sub-threshold level, where no measurable physiological response is observed. For instance, a sub-perception threshold in electromyography (EMG) corresponds to the absence of detectable EMG activity, while a sub-perception threshold in visual observation corresponds to the absence of any observable twitch, movement, or change in muscle tone. By selectively steering stimulation fields to achieve robust on-target activation while maintaining sub-threshold activity in non-target regions, the system provides a higher therapeutic benefit with fewer side effects.

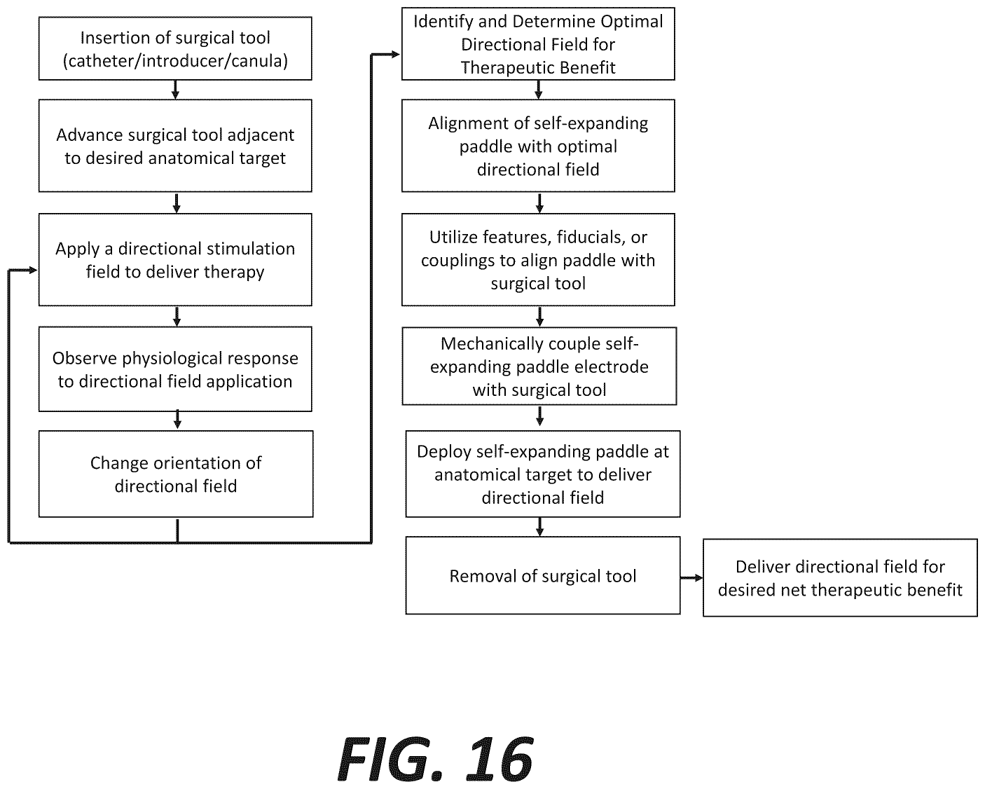

FIG. 17 illustrates a flowchart of an automated method of performing directional mapping and therapy electrode deployment using the catheter 20 in conjunction with a stimulation and feedback controller. It should be noted that this method is substantially simplified from a longer process that may normally be used. Accordingly, the method shown in FIG. 17 may have many other steps that those skilled in the art likely would use. In addition, some of the steps may be performed in a different order than that shown, or at the same time. Furthermore, some of these steps may be optional in some embodiments. Accordingly, the process is merely exemplary of one process in accordance with illustrative embodiments of the invention. Those skilled in the art therefore can modify the process as appropriate.

In various embodiments, the catheter is inserted and advanced to a target neural location under ultrasound guidance, and initial stimulation is delivered through the catheter's built-in electrode array. The system then automatically measures compound muscle action potentials (CMAPs) or other physiological signals such as electromyography (EMG) responses, and evaluates whether the resulting activity corresponds to on-target or off-target effects.

If the response is insufficient or produces off-target effects, the controller may automatically select another electrode or electrode pair within the catheter array and repeat stimulation. By sweeping across amplitudes, directions, and electrode combinations, the controller identifies the configuration that produces a robust on-target response with minimal or no off-target effects. Once an optimal orientation and electrode location are identified, the system records the alignment so that a permanent self-expanding paddle electrode may be deployed in the same orientation.

In certain embodiments, the controller may also incorporate algorithmic confirmation steps, such as applying bipolar stimulation to fine-tune the proximity field, or using a secondary confirmation source (e.g., SpO₂ or airflow monitoring) to verify that visual off-target effects are minimized. Following confirmation, the permanent paddle is mechanically coupled to the catheter, aligned with the recorded orientation, and deployed such that the directional therapy electrode delivers stimulation in the optimal orientation identified by the automated mapping workflow.

This embodiment demonstrates how the catheter 20 may be used in concert with computational tools to simplify intraoperative workflow, reduce operator dependency, and ensure that directional mapping results are translated consistently into permanent therapy.

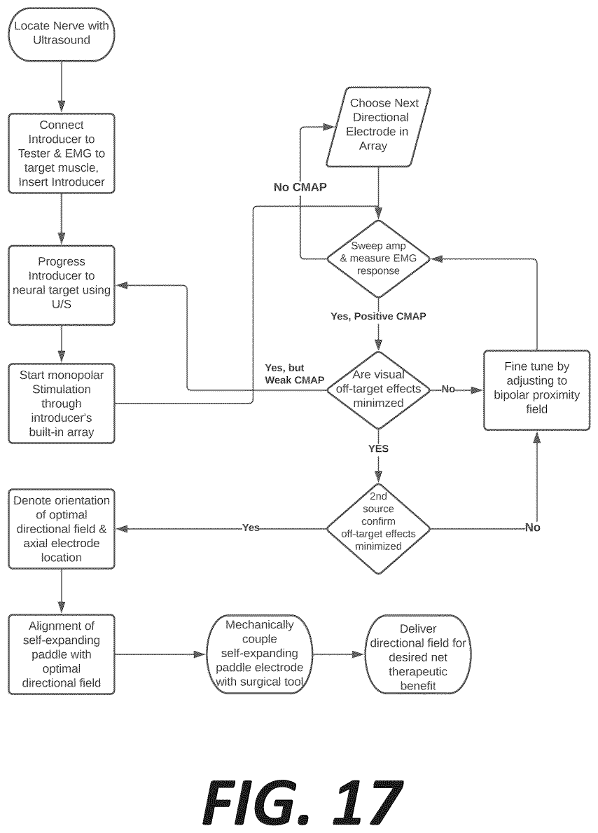

FIG. 18 illustrates anatomical examples where directional mapping is advantageous for therapeutic targeting. The catheter 20 is to be advanced proximate to a neural target, such as branches of the hypoglossal nerve adjacent to tongue musculature. The catheter may include distal electrodes or apertures for applying directional stimulation fields toward nearby tissue. By adjusting the orientation of the catheter relative to the neural anatomy, a physician may identify directions that preferentially activate on-target structures while minimizing activation of off-target structures. This figure highlights the clinical relevance of directionality in neuromodulation therapy, particularly for applications in which nearby motor branches can produce undesirable side effects if activated.

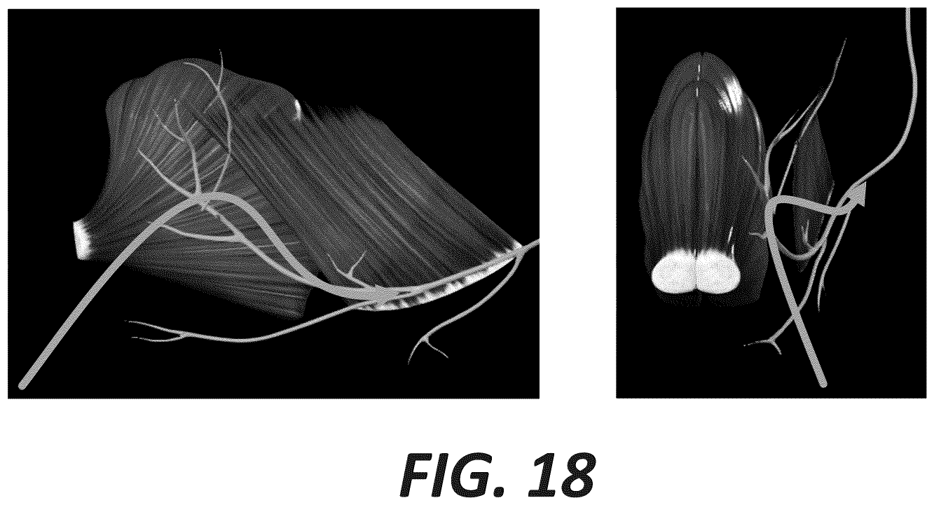

FIGS. 19-21 depict an exemplary workflow in which the catheter 20 aids in identifying the optimal directional field for therapy and subsequently delivers a self-expanding paddle electrode into the same trajectory.

FIGS. 18-21 illustrate representative treatment steps for applying a directional stimulation field to achieve a net therapeutic benefit, shown here in the context of a hypoglossal nerve stimulation embodiment. As shown in FIG. 18, the patient is positioned and prepared for treatment. In FIG. 19, a surgical tool is introduced with the goal of delivering directional stimulation to a selected nerve branch to produce a therapeutic response such as tongue protrusion. The surgical tool 20 includes one or more features configured to facilitate directional stimulation, such as integrated electrodes, apertures, or orientation fiducials.

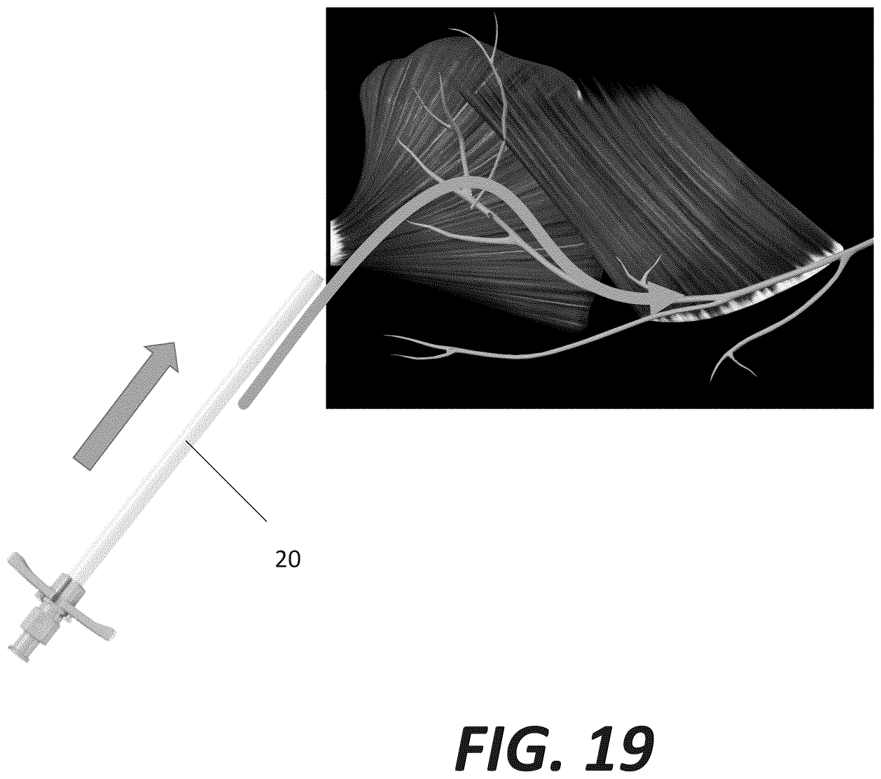

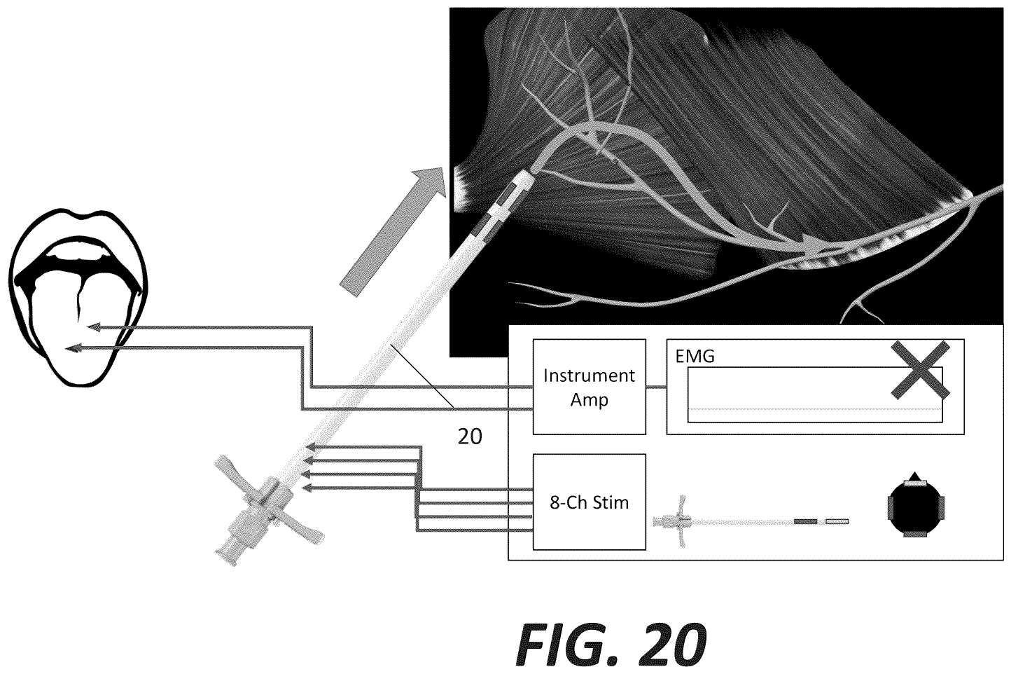

As shown in FIG. 20, in some cases the directional field applied by the surgical tool may fail to achieve a net therapeutic effect. For example, stimulation may recruit off-target muscles or fail to generate sufficient protrusion of the tongue to open the airway. In contrast, FIG. 21 illustrates the scenario in which the surgical tool successfully applies a directional stimulation field to the target anatomy, resulting in a desirable therapeutic effect. In this case, the directional field produces activation of the protrusor muscles while avoiding retrusor activity, thereby opening the airway and achieving the intended treatment benefit. Collectively, FIGS. 18-21 demonstrate how the disclosed system aids in applying and adjusting directional fields to identify an orientation that maximizes on-target therapeutic effects while minimizing or eliminating off-target effects.

Upon release, the paddle expands laterally, anchoring itself against surrounding tissue and maintaining the chosen stimulation direction. The catheter may then be withdrawn, leaving the paddle electrode in place to deliver long-term therapeutic stimulation. This sequence ensures that the directional therapy electrode is deployed in precisely the same angular orientation and axial location as determined during the intraoperative mapping phase. In certain embodiments, fiducials, keyed handles, or radiopaque markers may be used to preserve directional alignment between the catheter and the implanted paddle electrode.

Together, FIGS. 18-21 emphasize the integration of anatomical targeting, directional field mapping, and permanent lead deployment. By unifying these steps, the disclosed system enables minimally invasive delivery of paddle-like directional therapy without the need for open surgery or subsequent device manipulation.

FIG. 20 illustrates an embodiment in which the catheter 20 is equipped with a set of directional stimulation electrodes or apertures 72 near its distal tip. These electrodes may be selectively energized to generate localized test fields oriented in different angular directions. During mapping, the catheter is positioned adjacent to the neural target, and stimulation is delivered sequentially through different electrodes or electrode combinations. The physician observes physiological responses such as EMG activity or functional movement to identify the electrode orientation that produces a desirable therapeutic response while minimizing off-target activation.

In this embodiment, the catheter 20 serves as both a mapping tool and a delivery conduit. After the optimal directional orientation has been identified (e.g. in FIG. 21), the catheter is held in place to preserve both its angular orientation and axial position relative to the neural tissue. A self-expanding paddle electrode 14 is then advanced through the lumen of the catheter until it reaches the distal opening. The paddle electrode is oriented such that its active electrode array faces in the same angular direction (“north”) as the optimal mapping electrode identified during the initial testing phase.

The catheter 20 also provides for axial alignment. The catheter's distal electrode array provides a reference length (e.g., spanning a defined portion of the distal catheter). By advancing the paddle electrode until it occupies the same axial region that was tested, the physician ensures that the implanted paddle covers at least the same tissue volume that produced favorable mapping responses. In some embodiments, the paddle electrode may span a slightly greater axial distance than the mapping array, thereby ensuring robust coverage of the mapped region. Once positioned, the catheter is withdrawn while leaving the paddle electrode deployed at the optimal site.

This approach allows the permanent self-expanding paddle electrode to reproduce both the angular orientation and the axial position of the temporary directional fields generated by the catheter. In certain embodiments, fiducial markers, keyed insertion handles, or radiopaque bands may be used to confirm that the electrode is maintained in the mapped orientation throughout deployment. By combining directional angular control with precise axial positioning, the system ensures that the permanent electrode delivers therapeutic stimulation that faithfully reproduces the optimal field determined during intraoperative mapping.

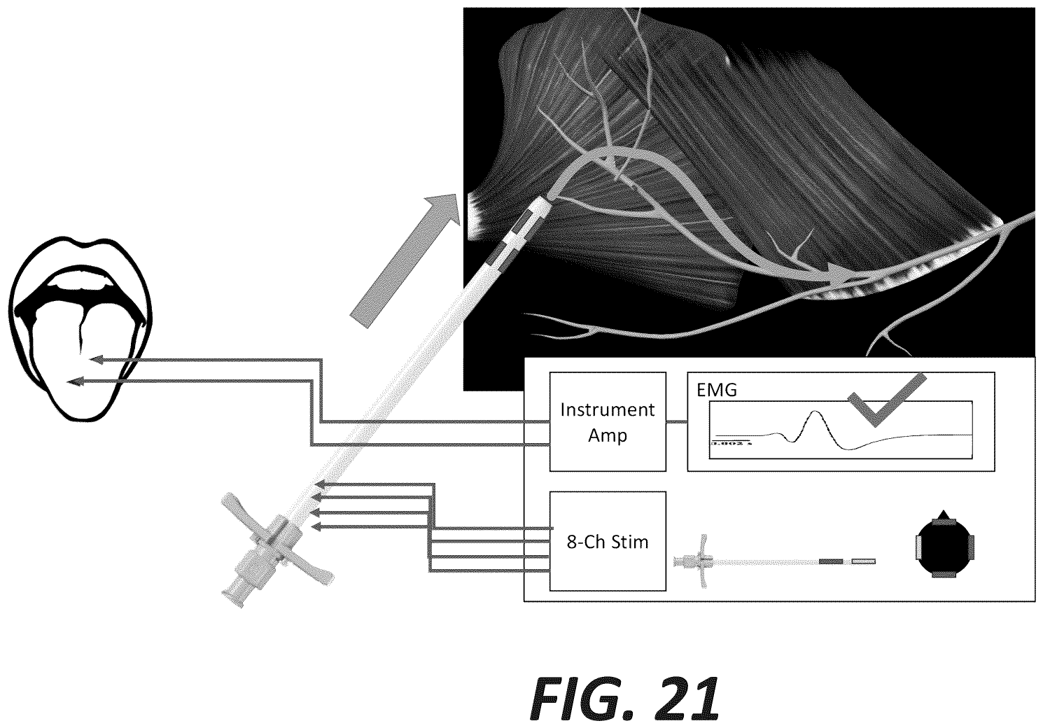

FIG. 22 illustrates an example of an intraoperative test report generated during use of the catheter 20. In this embodiment, the catheter's directional stimulation electrodes or apertures are sequentially activated while the physician monitors physiological responses to determine which orientation provides the most favorable therapeutic effect. The report may be displayed in real time on a surgical console or recorded for later reference, providing a clear visualization of both successful and unsuccessful electrode orientations.

As shown in FIG. 22, each row corresponds to a different directional configuration of the catheter 20. In the left-hand column, visual indicators (e.g., green checkmarks or red crosses) summarize whether the tested configuration yielded a desirable physiological response. The adjacent schematic views illustrate the catheter 20 with the selected directional electrode highlighted. The figure shows the corresponding orientation map, confirming which directional field was applied and whether it was therapeutically beneficial.

This reporting process allows the physician to rapidly compare multiple electrode orientations and converge on the optimal configuration. Importantly, once the most effective orientation is identified (the desired orientation, also referred as to the optimal orientation), the catheter 20 maintains its angular orientation in situ, ensuring that the subsequently deployed permanent electrode (e.g., a cylindrical segmented lead or self-expanding paddle electrode) is delivered in the same directional alignment. By preserving this orientation continuity, the system eliminates the need for repeated repositioning or rotation of the permanent lead after deployment, thereby reducing procedure time and minimizing patient risk.

In certain embodiments, the intraoperative test report may be integrated into the control system described above with reference to FIGS. 12, 13, and 30. The processor may automatically record the EMG or functional response associated with each directional electrode, assign pass/fail indicators, and highlight the orientation yielding the best therapeutic window. The report may further include axial position information, confirming not only which angular direction is optimal but also which longitudinal segment of the catheter's electrode array corresponds to the therapeutic site.

Thus, FIG. 22 represents one embodiment in which the catheter 20 not only aids in intraoperative mapping but also produces a structured output that guides final electrode deployment. This integration of mapping, reporting, and delivery within a single surgical tool further distinguishes the system from conventional introducers that serve only as passive conduits.

It should be apparent that in various embodiments, the catheter 20 facilitates both delivery of a therapy electrode 14 and intraoperative identification of an optimal stimulation orientation. The catheter 20 includes an elongate tubular body defining a lumen sized to receive a variety of implantable neuromodulation electrodes, including segmented cylindrical leads or self-expanding paddle electrodes 14. The tubular body further incorporates one or more directional stimulation features configured to generate, assist, or permit delivery of a directional stimulation field through the catheter wall. Such features may include apertures extending radially through the catheter wall, electrodes integrated or printed on the catheter's outer surface, or a conductive stylet or introducer core disposed within the lumen. In some embodiments, a plurality of electrodes or apertures are arranged circumferentially or axially along the catheter wall, allowing selective activation to deliver stimulation fields in different angular orientations.

Orientation consistency between the catheter and the therapy electrode may be preserved through fiducial markers 26, keyed handles, or radiopaque indicators integrated into the catheter body. These orientation features ensure that once a directional field is identified intraoperatively, the indwelling electrode delivered through the lumen will deploy in the same angular orientation, thereby eliminating the need for subsequent rotation or repeated stimulation mapping. The catheter may also incorporate staggered apertures, multiplexed electrode arrays, or rotatable stylets to provide multiple modes of directional stimulation and to reduce the mechanical strain that could otherwise occur in catheters with large continuous openings.

Unlike conventional catheters that serve only as passive delivery conduits, illustrative embodiments of the catheter 20 actively participate in intraoperative mapping by producing or aiding in the production of a directional stimulation field. This capability enables a physician to determine the most therapeutically beneficial orientation before implantation of the permanent electrode. In contrast, traditional procedures require placement of a directional electrode lead followed by iterative repositioning, rotation, or withdrawal/reinsertion of the catheter to confirm stimulation effects, steps that introduce error, prolong surgery, and increase the likelihood of off-target effects.

The catheter 20 may be used to generate a directional field through apertures 22, surface electrodes 12, or an internal conductive stylet 28 represents a significant departure from prior introducer designs, which typically lack integrated stimulation features. By combining delivery and mapping functions into a single tool, the present catheter reduces procedural complexity and provides physicians with immediate feedback on nerve selectivity and therapeutic efficacy.

The directional catheter 20 provides several advantages over prior art devices. First, it enables minimally invasive placement of electrodes with paddle-like directional benefits, avoiding the need for open surgical incisions traditionally required for paddle lead placement. Second, the catheter improves energy efficiency and therapeutic selectivity by allowing fields to be steered toward a target anatomy while avoiding adjacent off-target nerves or muscles. This reduces adverse side effects such as unwanted muscle contraction, pain, or airway closure.

Importantly, once the catheter identifies the preferred stimulation orientation, the therapy electrode is deployed in the same orientation without the need for further rotation or manipulation. This eliminates the trial-and-error workflow common to existing procedures, in which a physician must repeatedly rotate or reposition the electrode after deployment to achieve the desired physiological response. The one-step alignment ensures that the therapeutic configuration identified intraoperatively is preserved during long-term use, saving surgical time, reducing procedural complexity, and minimizing patient risk.

Finally, orientation alignment features (e.g., fiducials, keyed handles, radiopaque markers) and alternative embodiments (e.g., staggered apertures, printed electrodes, rotatable stylets) give the system flexibility in both manufacturing and clinical practice, while maintaining the inventive step of combining delivery and directional mapping in a single catheter.

In illustrative embodiments, the mapping system is configured to deliver a self-expanding lead 14 (FIGS. 31A-31B, 32, 33, 34A-34E, 35A-35D) into the same anatomical orientation identified by a temporary directional mapping tool. The self-expanding lead 14 includes a longitudinal body 110 and a substrate 120 that transitions between a compressed delivery configuration (FIG. 31A) and an expanded operational configuration (FIG. 31B). In the compressed configuration, the wings 125 of the substrate 120 are folded or curled about the longitudinal body 110 to minimize cross-sectional profile, enabling percutaneous introduction through a lumen of the catheter 20. Upon removal of the catheter 20, the normal bias of the substrate 120 urges the wings 125 outward into the extended configuration, thereby increasing surface area and exposing a plurality of therapy electrodes 130 toward a target tissue. While housed within the catheter 20, the wings 125 of the substrate 120 are biased inward toward the longitudinal body 110, thereby reducing the cross-sectional dimensions of the lead for percutaneous delivery.

After the catheter 20 is positioned proximate to a target neural tissue and an optimal stimulation orientation is identified using the catheter's directional stimulation features, the lead 14 is advanced distally within the lumen. Retraction of the catheter 20 relative to the lead 14 then permits the wings 125 to transition into the extended configuration (FIG. 31B), where the therapy electrodes 130 are oriented toward the mapped stimulation direction. Because the catheter 20 incorporates fiducials, keyed handles, or radiopaque indicators, the orientation of the lead 14 within the lumen is preserved, ensuring that the expanded paddle surface is deployed in the same angular orientation that was identified intraoperatively.

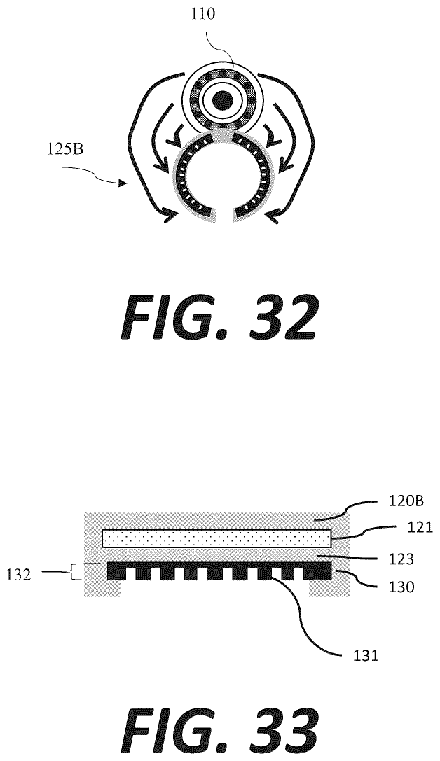

As shown in FIG. 33, the substrate 120 may incorporate an embedded urging layer 121, such as a shape-memory alloy or elastic polymer, which provides restorative force to bias the wings 125 toward the extended configuration. In some embodiments, the electrodes 130 are embedded within the substrate 120 to ensure consistent directional field orientation once deployed. The expanded geometry permits a paddle-like surface area while preserving minimally invasive delivery through a catheter, cannula, or introducer sheath. When released from the catheter 20, this urging layer 121 facilitates reliable self-expansion of the electrode surface without requiring physician manipulation. In some embodiments, the catheter 20 may further include aperture cutouts aligned with the electrodes 130 such that stimulation fields may be generated both during mapping and during deployment.

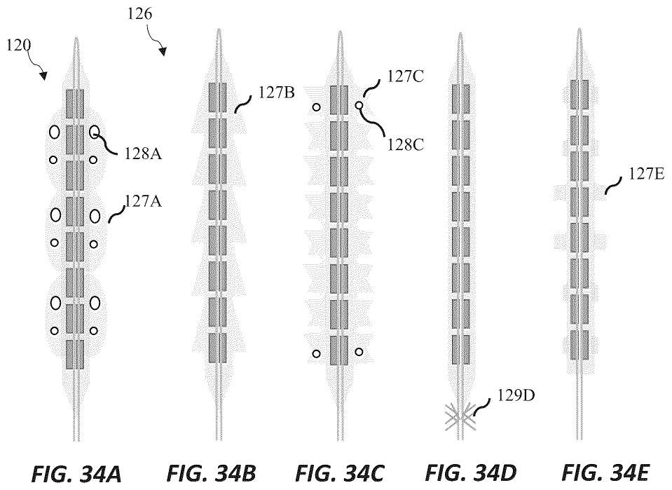

FIGS. 34A-34E illustrate anchor configurations 126 usable with the self-expanding lead 14. Such anchors may include edge features 127, hole features 128, or tines 129 that mechanically couple the lead 14 to surrounding tissue. By resisting longitudinal or latitudinal migration, the anchors 126 help maintain the therapeutic orientation that was identified intraoperatively during the mapping process. For example, a sawtooth edge feature 127B (FIG. 34B) may prevent retrograde displacement of the lead following deployment, thereby stabilizing the electrode surface relative to the mapped target nerve. These anchors may advantageously maintain the therapeutic orientation identified with the catheter 20, providing long-term stability of the stimulation field.

FIGS. 35A-35D schematically show one example of the deployment process. In operation, the self-expanding lead 14 is first housed in a lumen of the catheter 20 positioned proximate to the target tissue. After directional mapping identifies a desirable orientation, the catheter 20 is retracted, allowing the wings 125 of the substrate 120 to expand into the extended configuration (FIGS. 35C-35D). In the expanded state, electrodes 130 are directed toward the target neural structure in the same orientation confirmed during the mapping stage, enabling directional neuromodulation without further manipulation or open surgical access.

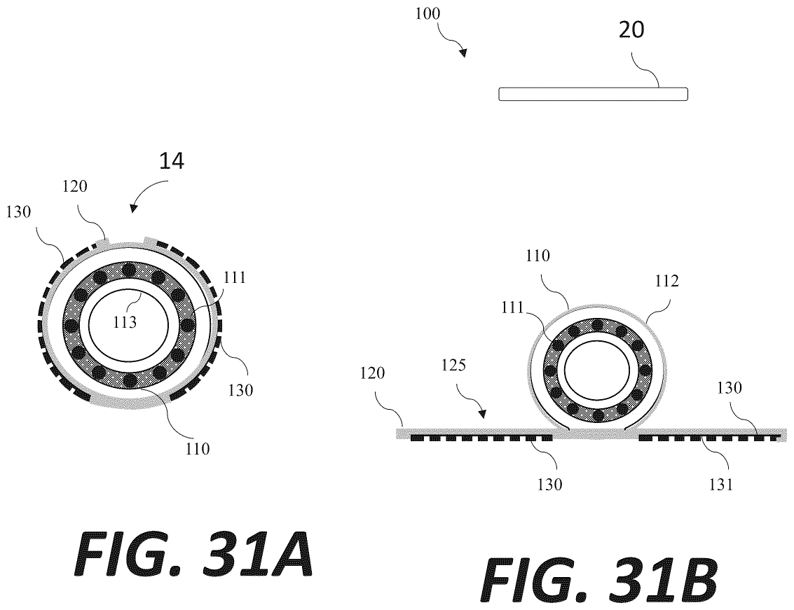

In some embodiments, the expanded paddle geometry may be configured to partially or fully encircle the target tissue, as shown in FIG. 32. This encircling wing configuration 125B provides enhanced coupling with peripheral nerves and can further focus stimulation fields in an inward radial direction. Such embodiments combine the energy efficiency and directional specificity of paddle electrodes with the minimally invasive percutaneous delivery facilitated by the mapping workflow.

In this way, the directional catheter 20 operates not only as a mapping tool but also as a delivery conduit for the self-expanding lead 14, preserving alignment between the temporary directional stimulation field and the final therapeutic electrode. This combination permits paddle-like directional therapy to be delivered percutaneously, eliminating the need for open surgical access.

### Exemplar Nerve Targets

The method of using a surgical tool to assist with the delivery of a directional stimulation field has application for multiple peripheral nerve targets that provide a therapeutic benefit for multiple diseases.

### Hypoglossal Nerve Stimulation

The HGN is a cranial nerve that controls the movement of the tongue. Disorders that affect the HGN can result in difficulty with speech, swallowing, and other functions. In some cases, direct access to the HGN is necessary for diagnostic or therapeutic purposes.

Illustrative embodiments provide a therapy method for optimal treatment of the HGN nerve using a directional stimulation field. More specifically, a directional stimulation field may be used to target and control specific tongue protrusor movement to open the airways to assist with breathing (e.g. in obstructive sleep apnea and sleep disordered breathing) while avoiding branches which activate tongue retrusors, which close the airways. The directional stimulation field must be identified and optimized for the indwelling therapy to be effective.

Current Limited Access to HGN. Traditionally, accessing the HGN has required surgical incision and open surgical field to apply a nerve-cuff electrode. The application of a nerve cuff often requires surgical dissection of the appropriate nerve branch (e.g., medial and lateral fibers must be separated to reach net positive effect), while only fitting the nerve-cuff around the branch that opens the airway (while avoiding branches that close the airway). The surgical procedure is known to have risks and complications.

Hypoglossal Anatomy. The HGN's complex branch anatomy begins at the hindbrain's motor nuclei, where the protrusor and retrusor cell bodies extend their axons to the specific protrusor muscle and retrusor muscles. When addressing upper airway obstructive sleep apnea, the focus lies on the protrusor muscles, which is known as the medial branches. By stimulating the protrusor branches of the HGN, a neural drive is provided to the tongue muscles. This neural activation helps in preserving the proper shape and position of the tongue, effectively preventing the tongue from obstructing the airway. Alternatively, if the nerve cuff contains lateral branches that activate retrusor muscles, then stimulation of the nerve may cause a push and pull effect. When both retrusor and protrusor muscles are simultaneously activated, their combined effect can limit the ability to displace the tongue away from the airway. This phenomenon is commonly known as a net effect with a goal of net positive unhindered protrusion of the stiffened tongue. Thus, the important process of separating the nerve branches surgically and identifying the protrusor and retrusor branches of the hypoglossal takes time and may injure the nerve. The nerve anatomy varies from subject to subject which can complicate the procedure and increase the time needed to dissect a nerve.

Therapy Effect of Stimulation. In a 3-dimensional space of peripheral nerve stimulation there are adjacent muscles and nerves that may provide on-target or off-target therapeutic effects. It is therefore important to identify the optimal direction of stimulation to maximize the benefit of electrical stimulation. For example, anatomical variations of the HGN can vary such that the critical nerve branches or segments required for tongue protrusion may not branch away from the nerve as expected. In 19% of patients, the transverse and vertical muscles can be easily identified as the uppermost portion of the distal HGN and the breakpoint between retractors and protrusors is easily identified. Whereas, in 44% of patients the transverse and vertical muscles are very close to the oblique genioglossus and horizontal genioglossus, and all superior branches are insinuated within a monolithic main medial branch of the HGN. This necessitates advanced physiological testing and measurement to identify the functional breakpoint of the protrusors vs retrusors. Finally, in 37% of patients, the transverse and vertical muscles are visually hidden. To ensure the correct minimally invasive deployment of a percutaneous lead with directional electrodes, there must be measurable on-target and off-target effects.

Off-Target and On-Target Effects from Adjacent HGN Nerve Branches. Multiple muscle groups are innervated by the HGN including the medial branch protrusor muscle fibers: medial inferior longitudinal, transverse, vertical, genioglossus oblique, and genioglossus horizontal. The HGN also innervates lateral branch retrusor muscle fibers that must also be avoided to prevent closing the airways: superior longitudinal, styloglossus, hyoglossus, lateral inferior and longitudinal. Additional muscles that are not innervated by the HGN are the geniohyoid (branch of C1), the anterior belly of the digastric, and the mylohyoid.

TABLE 1
Muscle fibers linked to the HGN and each fiber's associated
involvement in airway patency and tongue movement

| Muscle Fiber | Contribution | Nerve Origin | Action |
|---|---|---|---|
| Transverse | Protrusion | HGN-medial | Narrows tongue |
| Vertical | Protrusion | HGN-medial | Flattens tongue |
| Genioglossus | Protrusion | HGN-medial | Pulls tongue body |
| Oblique | | | downward |
| Genioglossus | Protrusion | HGN-medial | Pulls tongue forward |
| Horizontal | | | |
| Geniohyoid | Protrusion | C1 | Pulls hyoid toward chin |
| Styloglossus | Retrusion | HGN-lateral | Retracts and elevates |
| | | | tongue |
| Hyoglossus | Retrusion | HGN-lateral | Retracts and depresses |
| | | | tongue |
| Medial Inferior | N/A | HGN-medial | Shortens tongue and |
| Longitudinal | | | curls tip inferiorly |
| Superior | N/A | HGN-lateral | Shortens tongue and |
| Longitudinal | | | curls tip superiorly |
| Lateral Inferior | N/A | HGN-lateral | Shortens tongue and |
| longitudinal | | | curls tip inferiorly |
| Anterior belly | N/A | V3 | Elevates hyoid and |
| of digastric | | | depresses mandible |
| Mylohyoid | N/A | V3 | Elevates hyoid and |
| | | | tongue |

Those skilled in the art will understand that the HGN arborizations are known to provide therapeutic and anti-therapeutic effect by controlling the tongue in a positive therapeutic effect (protrusion) while also opposite of the desired therapeutic effect (retrusion). Intraoperative testing with electrical stimulation demands there to be primary and secondary feedback mechanisms to verify that the correct branch of the HGN is electrically isolated.
An example use-case is positively affecting Obstructive sleep apnea with on-target effects such as tongue protrusion, muscle stimulation, or airway patency obstruction clearing:

TABLE 2
On-Target/Off-targets for apnea

| Positive apnea-related effects | | Negative apnea-related effects | |
|---|---|---|---|
| 1. | Tongue Protrusion | 1. | Tongue retrusion |
| 2. | AHI reduction | 2. | Airway collapse |
| 3. | SpO2 | 3. | Loss of tongue shape |
| 4. | Chest wall movement | | |
| 5. | Tongue stiffening/firmness | | |

In various embodiments, practitioners may use an EMG readout connected to the tongue, or an endoscope to visualize the tongue protrusion vs retrusion, or a plethysmograph that can see maximum blood-oxygen levels that relate to the breathing efficacy while dormant (FIG. 14, FIG. 15). The EMG readout may be observed during orientation of the catheter, electrode, or self-expanding electrode.

Surgical Access to HGN. The HGN is typically accessed surgically by making a 4-6 cm (1.6-2.5 in) incision along a natural skin crease from 3-4 cm (1.2-1.6 in) below the right edge of mandible. The submandibular gland is retracted cephalad. The digastric muscle is identified, and carefully dissected in the submandibular triangle to identify the HGN near the hyoglossus muscle. The majority of fibers within the human HGN spread extensively between the posterior and anterior borders of the hyoglossus muscle. This anatomical transformation from a predominantly single-fiber nerve to a multifaceted branching structure makes the hyoglossus muscle a pivotal point for anatomical targeting. It plays a crucial role from the initial incision planning to the identification of individual branches of the polyfascicular distal nerve. This identification helps determine which branches of the HGN will be included in or excluded from the stimulation cuff electrode.

### E.g., Method of Optimal HNS for Airway Opening

Stimulation is activated and nerve target effects are monitored through tongue EMG, tongue force, tongue protrusion, tongue movement (accelerometer), twitch visualization, endoscopy, neuromonitoring, or direct patient feedback. The directionality of the sheath's exposed electrode can be observed by locating the fiducials. In some embodiments, the tongue is connected to an EMG to observe a positive Compound Motor Action Potential (CMAP) response. If the CMAP is weak or intermittent, the electrodes can be rotated or translated to achieve more optimal coupling. Stimulation may also be evaluated in a closed-loop fashion using Evoked Compound Action Potentials (ECAPs). In some embodiments, intraoperative testing may include an endoscopic camera that is used to visualize the tongue's direction of contraction during stimulation. If the genioglossus muscle of the tongue is generating EMG signals, but is not protruding, then the stimulating directional electrodes may need to be adjusted rotationally or axially to stimulate the correct arborization branch of the HGN (such that protrusors and retrusors are not cancelling one another). When adjusted correctly, the protrusor EMG should be positive and without retrusor EMG. Also, tongue visualization should confirm protrusion instead of retraction with concomitant airway opening. In other embodiments, the blood-oxygen levels may be monitored to validate airway patency while testing electrode placement. In other embodiments, airway opening may be measured through a pneumotachometer or modified PAP by evaluating air volume or pharyngeal critical closing pressure (Pcrit).

### Other Peripheral and Complex Nerve Targets

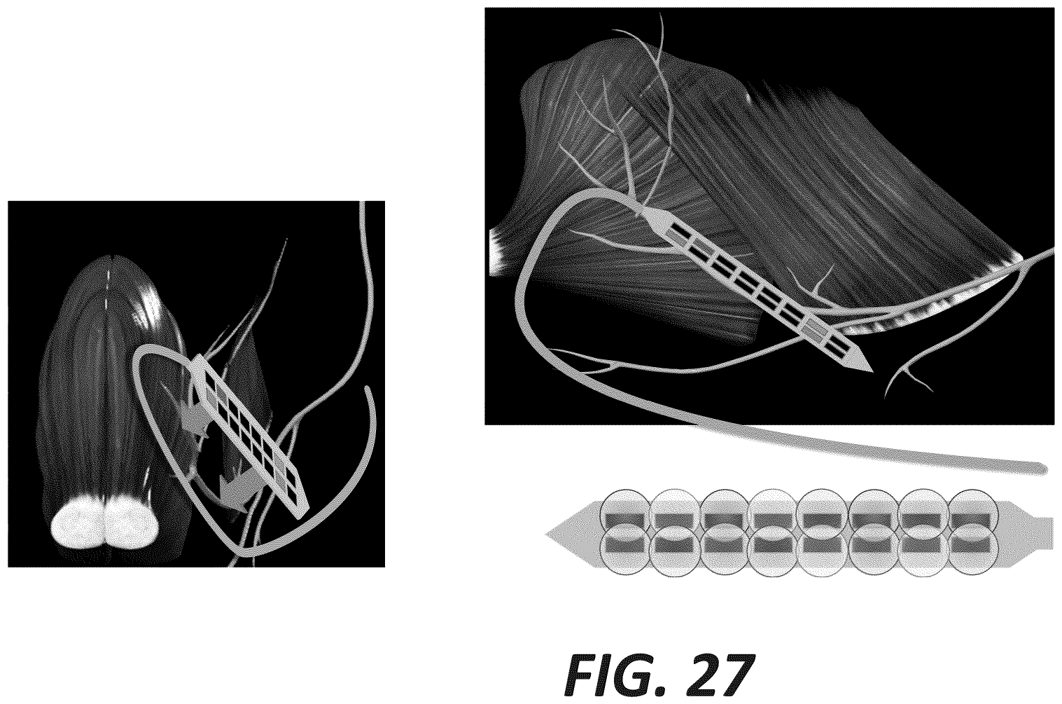

Stimulating the pudendal nerve (FIG. 28) with a directional stimulation field involves targeting specific nerve fibers to improve motor and sensory functions in the pelvic region. By using focused, directional currents, clinicians can selectively activate areas responsible for urinary, bowel, and sexual functions without affecting nearby tissues. This technique is valuable in treating conditions like urinary incontinence, pelvic pain, and erectile dysfunction. It also allows for more precise control over the intensity and localization of stimulation, reducing side effects. Directional stimulation offers a promising approach for enhancing outcomes in neuromodulation therapies for pelvic disorders.

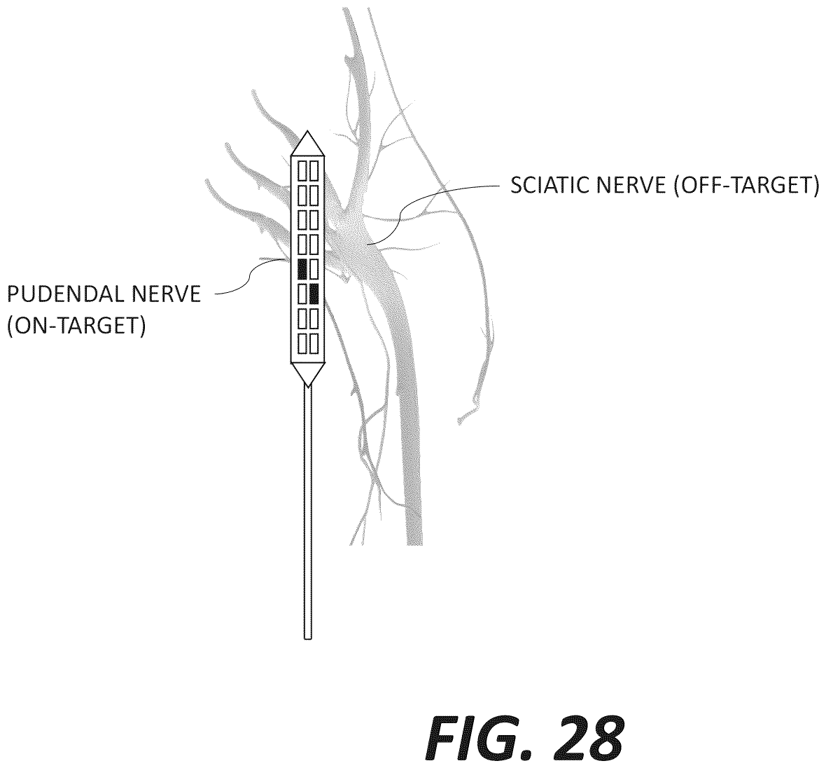

Stimulating the carotid sinus nerve (FIG. 29) with a directional stimulation field focuses on modulating baroreceptor activity to regulate blood pressure. This technique targets specific nerve fibers that control cardiovascular responses, allowing for precise regulation of blood pressure without influencing surrounding tissues. It has therapeutic potential for treating conditions like hypertension and heart failure by improving autonomic control over heart rate and vascular tone. Directional stimulation offers more refined control compared to traditional methods, minimizing unwanted effects such as bradycardia or hypotension. As a result, it could enhance the efficacy and safety of neuromodulation in cardiovascular therapy.

### Automated Algorithms for Optimum Therapy Coupling

In some embodiments, the feedback and orientation process are automated by a control system (FIG. 13) that automatically determines the correct deployment orientation of a lead with directional electrodes. In some embodiments, the automated control system may monitor one or more EMG signals or other biofeedback signals that contain a threshold or envelope detector to automatically verify on-target versus off-target effects (FIG. 17).

In some embodiments, the process of identifying an optimal stimulation orientation is automated by a control system that integrates with the surgical tool and therapy electrode. As illustrated in FIG. 13, the system may include a processor, memory, and signal analysis circuitry configured to monitor biofeedback signals during intraoperative stimulation. The control system evaluates these signals in real time to determine whether a stimulation field is producing a therapeutic effect, an off-target effect, or a combination of both. By automating this evaluation, the system reduces reliance on subjective physician interpretation and accelerates the workflow of mapping and electrode deployment.

The automated system may monitor one or more electromyography (EMG) signals recorded from target and non-target muscles. In certain embodiments, the EMG traces are analyzed using threshold detection, envelope detection, or frequency-domain analysis to distinguish between desired activations (e.g., protrusor muscle contraction) and undesirable activations (e.g., retrusor muscle contraction). When the system determines that a stimulation configuration yields an EMG response meeting a predefined therapeutic threshold without exceeding an off-target envelope, the configuration is classified as optimal. In other embodiments, the system may combine EMG analysis with additional physiological signals, such as plethysmography, respiratory airflow, blood oxygenation, or evoked compound action potentials (ECAPs), thereby providing a multimodal assessment of therapy efficacy.

As an example, during intraoperative mapping of the hypoglossal nerve, a stimulation field is applied through a catheter with directional electrodes. EMG signals are simultaneously recorded from both the genioglossus (tongue protrusor) and the styloglossus (tongue retrusor). The automated system is programmed with a predefined therapeutic threshold:
On-target condition (therapeutic): a genioglossus EMG amplitude exceeding 200 μV sustained for at least 50 ms.
Off-target condition (undesirable): a styloglossus EMG amplitude exceeding 100 μV.

When the stimulation field oriented in the “north” direction produces a genioglossus EMG response of 250 μV with no measurable retrusor activity above 50 μV, the system classifies this as meeting the therapeutic threshold. Conversely, when the field is oriented “east” and produces 280 μV protrusor activity but also 180 μV retrusor activity, the configuration is rejected because it fails the off-target exclusion criterion.

The control system may iterate through different directional stimulation fields by selectively energizing electrodes, activating apertures, or mechanically rotating a stylet or cannula. For each orientation, the feedback data are captured and compared against stored criteria. The algorithm may then automatically select the stimulation orientation that produces the highest therapeutic benefit with the lowest off-target activity. Once an optimal orientation is identified, the system may lock this configuration, trigger deployment of the therapy electrode, or provide visual or audible cues to the physician confirming the orientation for implantation.

In one example, a catheter may incorporate multiple electrode segments distributed circumferentially around its outer surface. For instance, four discrete electrodes may be positioned at 0°, 90°, 180°, and 270° around the catheter wall. During intraoperative mapping, the system may sequentially energize each electrode segment while recording corresponding physiological responses, such as electromyography (EMG) activity. By comparing the therapeutic and off-target responses produced by each energized segment, the system or physician may identify the angular orientation that delivers the most desirable stimulation. This selective electrode energization allows the catheter itself to apply a directional stimulation field without requiring mechanical rotation.

In another example, the catheter or cannula may include one or more apertures formed in its sidewall. A conductive stylet or electrode within the lumen may emit current, but the field is directed outward through the aperture, thereby restricting stimulation to a defined angular region. Multiple apertures may be staggered or offset axially along the catheter wall, with each aperture selectively activated by a switch or multiplexing circuit. For example, activation of a superior aperture produces a stimulation field oriented upward, whereas activation of a lateral aperture directs the field sideways. Cycling between apertures allows the operator or automated system to rapidly evaluate multiple field orientations without repositioning the catheter.

In a further example, the catheter may incorporate a partially insulated stylet having a conductive window or exposed region along one side. When the stylet is rotated within the lumen, the conductive window changes angular alignment relative to the surrounding anatomy, redirecting the stimulation field in discrete increments. In one embodiment, the stylet may be rotated by 45° or 90° steps, with EMG or other physiological responses recorded at each angular orientation. This technique allows fine mapping of on-target and off-target responses using a single stimulating element, while leveraging the catheter's lumen and insulation to confine and steer the directional stimulation field.

This automated feedback and orientation process provides several advantages. First, it standardizes intraoperative mapping across operators, reducing variability due to physician experience or interpretation. Second, it minimizes procedure time by rapidly converging on an optimal orientation without iterative manual adjustments. Third, it enhances precision by using quantitative biofeedback measures that are less prone to error than subjective assessments. Importantly, the automation allows the final implanted electrode to be confidently aligned with the orientation identified by the mapping tool, ensuring consistent therapeutic benefit while reducing the need for post-deployment repositioning.

Illustrative embodiments provide a method of applying a directional stimulation field and the determination of the optimal stimulation field aided by a surgical tool for delivering therapy. The method uses a directional stimulation field near a nerve or nerve branch, applies electrical stimulation to an electrode, and observes or measures the therapeutic response to stimulation (e.g., tongue movement distance or force, tongue protrusion, retractor, airway opening, tongue electromyography (EMG), muscle movement, etc.). After the response is observed, the directional stimulation field may be altered until an optimal therapeutic response is obtained (FIG. 12). This iterative method of applying a directional field aided by a surgical tool and optimizing the effect of stimulation for maximum therapeutic benefit, and application of a directional field by the indwelling implant is the method of this patent. Critical to the method of delivering a directional stimulation field is the use of, at the time of implant, a surgical tool (e.g., catheter, cannula, guidewire, etc.) that facilitates orienting the direction of the stimulation field in a desired direction.

Some embodiments of the method describe the direction of the stimulation field being altered or changed by activating one or more stimulation electrodes or by mechanical rotation of a stimulating element relative to the biological tissue.

In illustrative embodiments, a method of delivering a directional neuromodulation therapy electrode utilizes a cannula having one or more apertures and fiducial features that enable controlled rotation and orientation of a directional stimulation field during implantation. As illustrated in FIG. 18, the patient may be positioned in a suitable manner, such as a supine position with the head slightly turned to one side, and the skin overlying the target anatomy is prepared in a sterile fashion. A primary feedback monitoring system, such as electromyography (EMG) electrodes positioned on protrusor and retractor muscles of the tongue, is connected and observed during the procedure. Secondary feedback systems may be used concurrently, such as a plethysmograph to measure blood oxygenation, to provide additional indicators of therapeutic efficacy.

Using image guidance, such as ultrasound or fluoroscopy, the surgeon identifies the target nerve arborization and maps out a percutaneous approach. A cannula incorporating directional apertures is advanced into the tissue and placed adjacent to the target neural structure under imaging control. A lead with directional electrodes may be inserted into the lumen of the cannula for intraoperative testing. Fiducial markers associated with the cannula allow the angular orientation of the apertures, and therefore the applied stimulation field, to be tracked relative to anatomical landmarks.

After the cannula is in place, intraoperative testing begins by connecting the electrodes to a pulse generator. The directional stimulation field is delivered through the apertures of the cannula and monitored in real time. The surgeon evaluates stimulation efficacy by observing the feedback systems, which may include EMG traces of protrusor and retrusor muscles, changes in tongue shape or movement, tongue force, surface EMG from suprahyoid and masseter muscles, visible twitches, verbal feedback from a the patient, neuromonitoring data, or airway-related measurements such as air volume or pharyngeal critical closing pressure (Pcrit). As shown in FIGS. 20 and 21, these responses allow the operator to determine whether stimulation produces desired on-target effects (e.g., tongue protrusion and airway patency) or undesirable off-target effects (e.g., tongue retrusion and airway collapse).

If off-target effects are detected, the cannula is rotated, advanced, or retracted to fine-tune the directional stimulation field. The process of iteratively rotating the cannula while monitoring the corresponding feedback is illustrated in FIG. 22. This iterative adjustment continues until a directional field is identified that maximizes on-target effects and minimizes off-target activation. In some embodiments, a stimulation feedback controller may be employed to automate the selection of electrodes and directional orientation based on the monitored physiological responses.

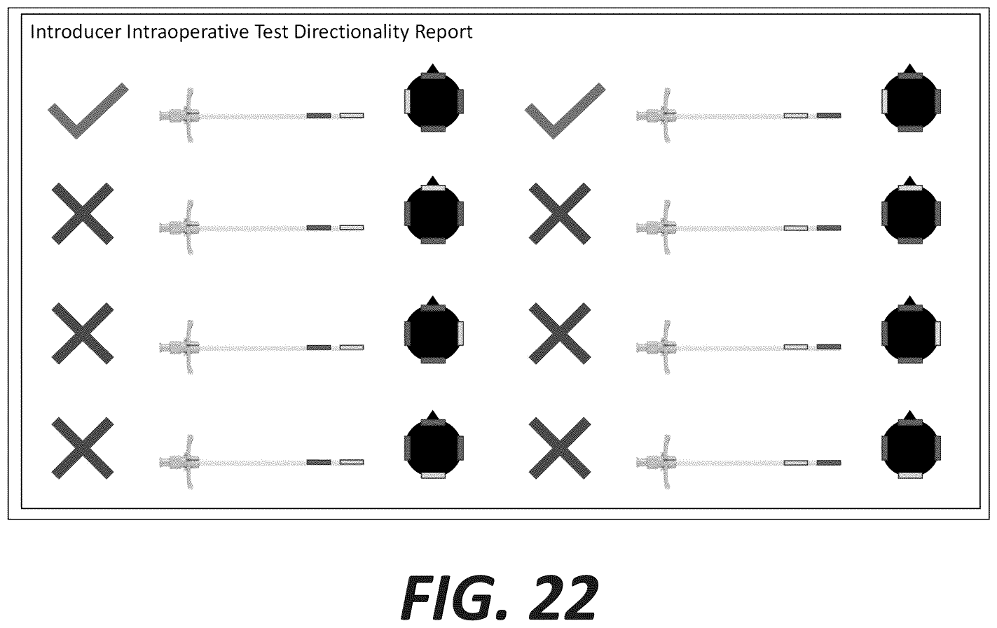

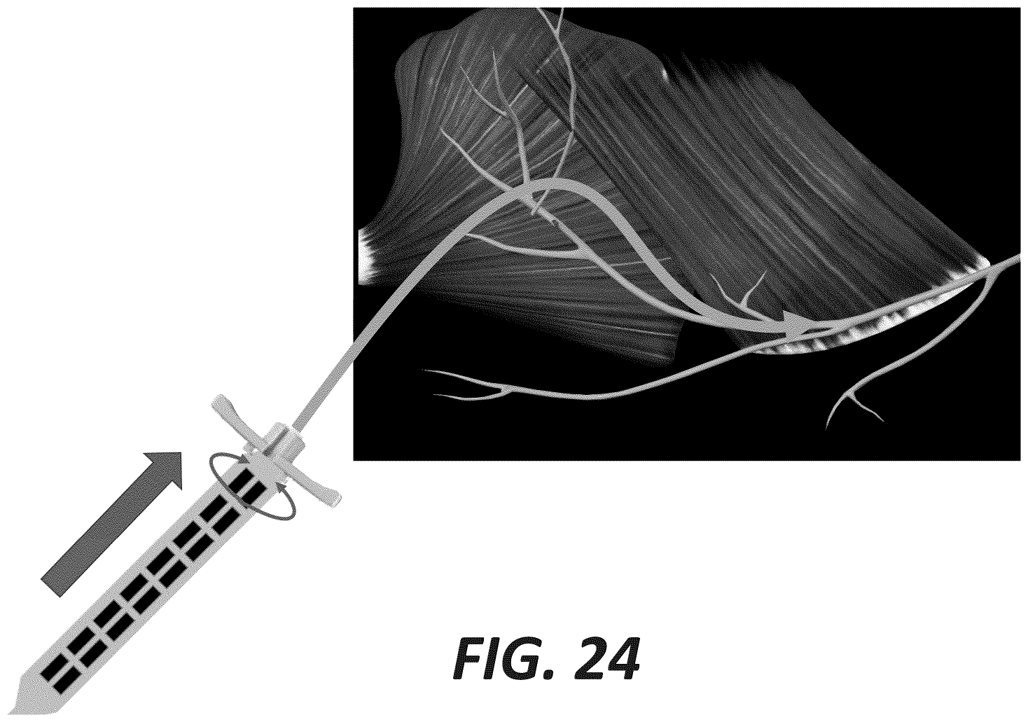

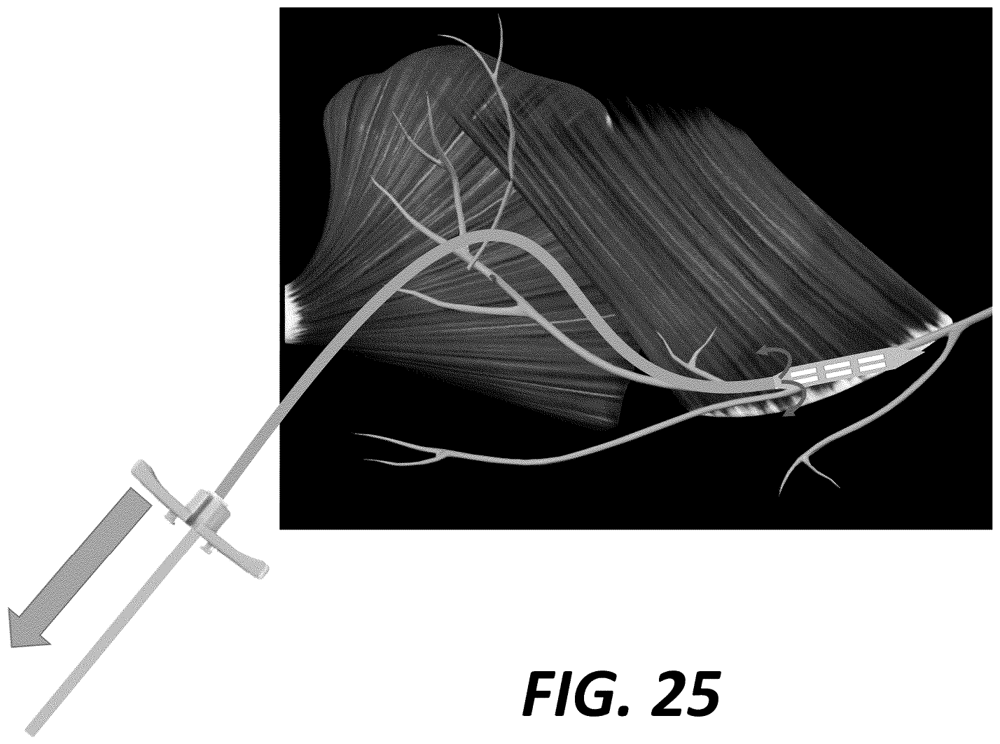

Once the optimal stimulation direction is determined, the therapy lead is rotated within the cannula so that its directional electrodes or self-expanding paddle wings align with the identified orientation. As depicted in FIG. 23, the lead is then deployed in the confirmed orientation, ensuring its primary stimulation region is directed toward the therapeutic target. Following deployment, the cannula is withdrawn (FIG. 26), leaving the lead anchored in place (FIG. 25). The expanded paddle or electrode wings provide stable coupling to the tissue, maximize directional field coverage, and reduce stimulation of off-target regions.

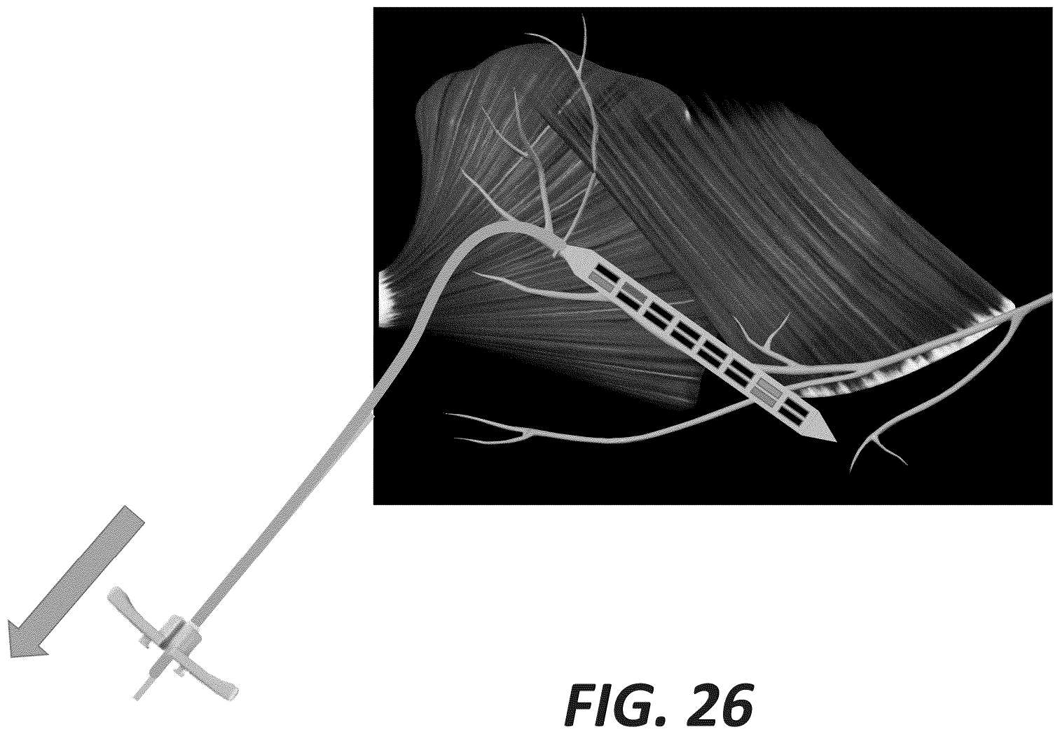

The lead may be retested intraoperatively to confirm delivery of the desired physiological response (FIG. 27). If necessary, the lead can be adjusted through small rotations, axial translations, or manipulations to further optimize the directional field. For temporary trialing, the lead may be secured externally at the skin surface, while for permanent implantation the lead is anchored to fascia and connected to an implanted pulse generator through the incision site or via tunneling to a secondary pocket. The surgical sites are then dressed and bandaged to complete the procedure. This method advantageously allows physicians to identify the optimal stimulation orientation before final electrode deployment, using the cannula as both a delivery conduit and a directional mapping tool. The combination of apertures for shaping the field and fiducials for orientation tracking ensures that the directional field observed intraoperatively corresponds precisely to the final electrode orientation. As a result, the need for repeated post-deployment electrode rotation is eliminated, surgical time is reduced, and therapeutic selectivity is improved. Furthermore, because the cannula permits percutaneous access and orientation mapping, the method avoids the morbidity of open surgical paddle lead placement while achieving paddle-like directional benefits, including energy efficiency, stable anchoring, and reduced off-target stimulation.

In some embodiments, the patient is positioned appropriately for neuromodulation therapy, such as in a supine position with the head turned slightly to the side, as shown in FIG. 18. The skin overlying the target nerve is cleaned and sterilized, and feedback systems are established. A primary feedback system, such as an electromyography (EMG) electrode, is connected to a target muscle and displayed in real time. A secondary feedback system, such as a second EMG channel or a plethysmograph, may also be applied to evaluate broader physiological responses and confirm therapeutic benefit.

Using image guidance, such as ultrasound or fluoroscopy, the operator identifies the arborization of the target nerve and maps out a percutaneous pathway. A cannula containing one or more integrated directional electrodes on its outer circumference is then advanced into the target area adjacent to the nerve. The cannula is coupled to an external pulse generator, and stimulation is initiated. The applied directional stimulation field is tracked through fiducial markers integrated with the cannula, which indicate the orientation of the field relative to the surrounding anatomy.

During intraoperative testing, the therapeutic efficacy of stimulation is evaluated using the feedback systems. As shown in FIGS. 20 and 21, the surgeon may observe protrusor and retrusor EMG activity, surface EMG of suprahyoid or masseter muscles, twitch visualization, neuromonitoring, patient-reported feedback, or other physiological cues to distinguish on-target from off-target effects. If the recorded effects are suboptimal, alternative electrode combinations may be selected, or the cannula may be rotated to adjust the orientation of the directional field, as demonstrated in FIG. 22. This iterative process continues until the optimal orientation is determined, maximizing on-target effects and minimizing off-target recruitment.

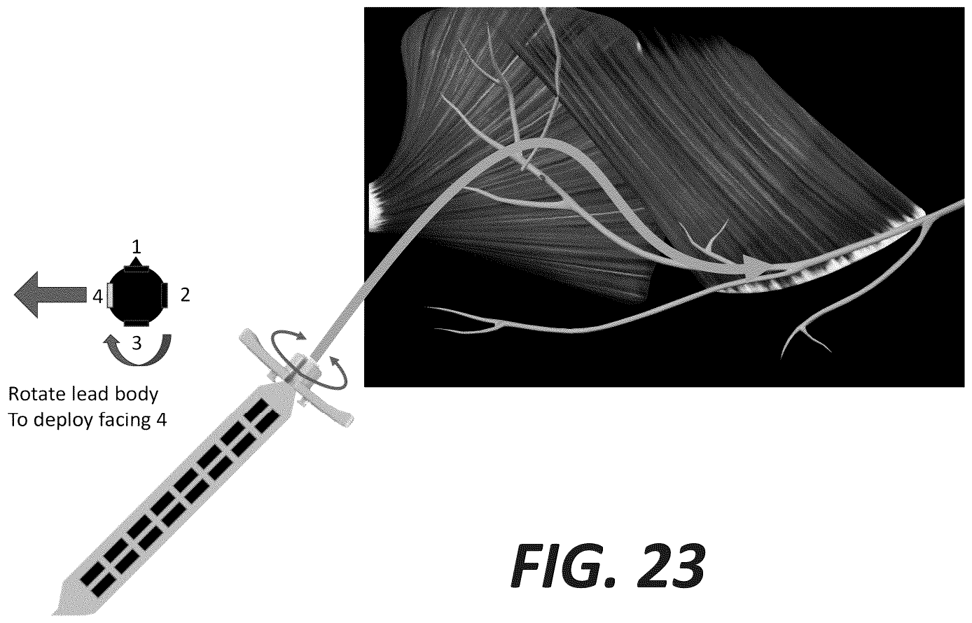

After the ideal directional stimulation field is identified, a permanent therapy electrode is prepared for deployment. In one embodiment, a self-expanding paddle lead with directional electrodes is oriented according to the results of the mapping procedure, ensuring that the deployed paddle directs energy toward the desired nerve while insulating off-target tissue, as depicted in FIG. 23. The therapy lead is mechanically coupled with the cannula and advanced into the target anatomical location, as illustrated in FIG. 24. Following deployment, the cannula is withdrawn from the patient (FIG. 26), leaving the lead implanted in situ with its directional field fully deployed (FIG. 25).

To confirm efficacy, the implanted lead may be tested intraoperatively to validate that it delivers the desired stimulation pattern and therapeutic muscle recruitment, while minimizing off-target activation, as shown in FIG. 27. If necessary, the lead may be adjusted through minor rotations, axial translations, or other manipulations to optimize the field orientation. For temporary trialing, the lead may be secured externally at the skin surface. For permanent implantation, the lead is anchored to nearby fascia using sutures or a dedicated anchoring mechanism. Finally, the lead is connected to an implanted pulse generator, either at the first incision site or through tunneling to a secondary incision, after which the surgical sites are dressed and bandaged to complete the procedure.

In illustrative embodiments, the electrophysiology lead system comprises a lead including a longitudinal body, a substrate, and an electrode. The longitudinal body may house one or more conductors for transmitting electrical signals between an external source and the electrode. The electrode is configured to deliver or record electrical signals from target tissue such as neural or cardiac structures. The electrode may include a nonuniform thickness or a discontinuity, enabling it to compress with the substrate during delivery and to expand reliably during deployment. In certain embodiments, the electrode includes a thickness between approximately 2 microns and 200 microns, permitting sufficient flexibility to withstand repeated compression while also maintaining structural robustness for long-term implantation.

In some embodiments, the nonuniform thickness is realized by an indentation extending partially through the electrode. The indentation provides a localized region of reduced stiffness that facilitates bending during compression into a catheter or sheath. In other embodiments, the discontinuity may comprise one or more voids extending completely through the thickness of the electrode. Such voids may be circular, elongated, staggered, or patterned to achieve a desired flexural response. Both indentations and voids may function as bending zones, reducing mechanical strain on the electrode during transition between compressed and extended configurations.

The substrate supporting the electrode is normally biased toward the extended configuration. This bias may be achieved using materials or structures that exhibit elasticity, super-elasticity, shape-memory behavior, thermal or phase-change responses, or polymeric/volumetric expansion. For example, a nitinol urging layer embedded in the substrate may provide shape-memory expansion, or a polymer such as polyurethane may provide elastic rebound. In this way, when the compressive restraint of the catheter or sheath is removed, the substrate urges the electrode outward, establishing a stable and predictable extended configuration.

To aid insertion and controlled compression, the substrate may include geometric guides. A distal guide, for example, may taper the substrate to direct it into the compressed configuration as it enters a lumen. A proximal guide may serve a similar function when retracted into the lumen for repositioning or removal. These guides may be integral polymer tapers, metallic spines, or cut patterns that facilitate uniform folding and minimize kinking.

In some embodiments, the lead includes a plurality of electrodes distributed along the substrate. When the lead is deployed into the extended configuration, these electrodes may be oriented in the same direction to generate a preferred stimulation field. Such alignment enhances directional control, reduces the required energy for stimulation, and minimizes activation of non-target tissues. Alternatively, in multi-wing configurations, electrodes may be arranged to encircle a nerve or direct fields along multiple vectors.

The substrate may further incorporate one or more anchors configured to couple the lead to tissue. Anchors may include edge features such as sawtooth ridges, curved flanges, or rectangular protrusions, each configured to resist migration in one or more directions. Additional anchors may include perforations or hole features that permit tissue ingrowth, or tines positioned on the longitudinal body to stabilize the implant against displacement.

To facilitate imaging and surgical navigation, radiopaque markers may be coupled to the substrate, the longitudinal body, or the electrodes themselves. These markers enable visualization of the lead's orientation and position during fluoroscopy, radiography, or ultrasound. In some embodiments, radiopaque markers are placed at the proximal and distal ends of the substrate to confirm that the lead has successfully transitioned to its extended configuration.

In various embodiments, a method of electrically coupling a lead to neural tissue includes directing the lead into a compressed configuration. During this step, the electrode, including its nonuniform thickness or discontinuity, flexes into a reduced cross-sectional dimension for delivery through a lumen. Once the lead is positioned adjacent the target tissue, the method transitions the lead to an extended configuration by removing the compressive force of the lumen. The normal bias of the substrate urges the electrode outward, presenting a larger surface area to the target and orienting electrodes in a preferred direction.

Guides incorporated into the substrate may direct the lead into the compressed configuration as it enters the lumen, ensuring controlled folding. Anchors on the substrate may then mechanically couple the extended lead to the biological tissue, preventing migration during patient movement. In some embodiments, radiopaque markers are used intraoperatively to confirm alignment and orientation of the lead before final deployment.

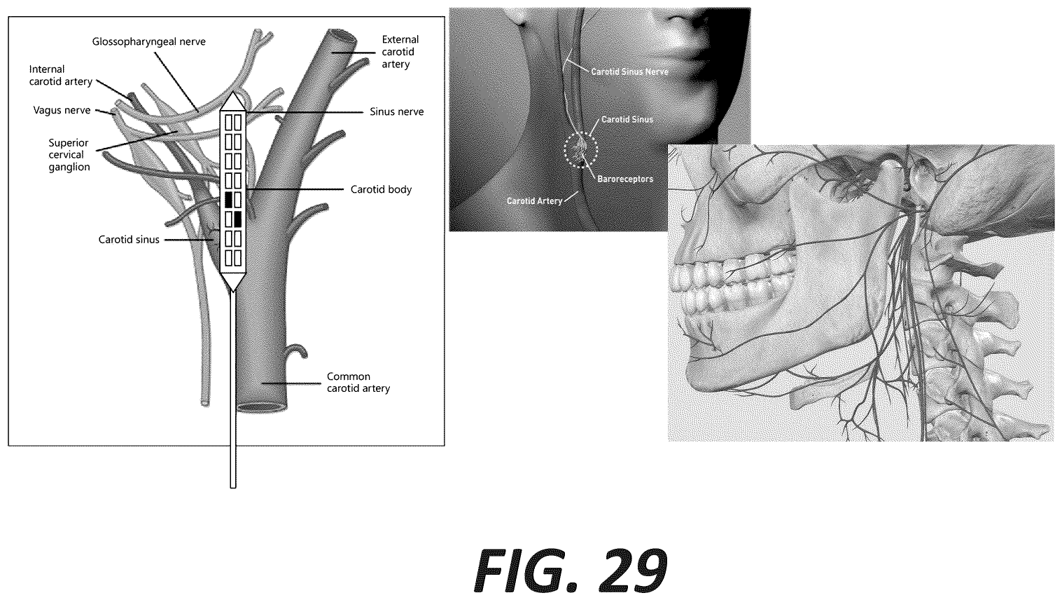

FIG. 30 schematically shows details of controller of the system configured in accordance with illustrative embodiments of the invention. Each of these components is operatively connected by any conventional interconnect mechanism. FIG. 30 simply shows a bus communicating each the components. Those skilled in the art should understand that this generalized representation can be modified to include other conventional direct or indirect connections. Accordingly, discussion of a bus is not intended to limit various embodiments.

Indeed, it should be noted that FIG. 30 only schematically shows each of these components. Those skilled in the art should understand that each of these components can be implemented in a variety of conventional manners, such as by using hardware, software, or a combination of hardware and software, across one or more other functional components. For example, the directional mapping logic (discussed in detail below) may be implemented using a plurality of microprocessors executing firmware. As another example, the feedback acquisition module may be implemented using one or more application specific integrated circuits (i.e., “ASICs”) and related software, or a combination of ASICs, discrete electronic components (e.g., integrated circuits), and microprocessors. Accordingly, the representation of the directional mapping logic and other components in a single box of FIG. 30 is for simplicity purposes only. In fact, in some embodiments, the power controller of FIG. 30 is distributed across a plurality of different components, not necessarily within the same housing or chassis.

It should be reiterated that the representation of FIG. 30 is a significantly simplified representation of the controller. Those skilled in the art should understand that such a device has other physical and/or functional components, such as central processing units, other packet processing modules, and short-term memory. Accordingly, this discussion is not intended to suggest that FIG. 30 represents all of the elements of the controller. In fact, much of what was said here with regard to FIG. 30 can also be applied to components of the system. Additionally, one or more of the components shown in FIG. 30 are optional in various embodiments.

FIG. 30 shows an external trial stimulator 50 operatively coupled to the surgical tool 20. However, it should be understood that in some embodiments, the processor may be coupled within the housing of the surgical tool (e.g., in a handle). Accordingly, discussion of modules within the external trial stimulator is also intended to include discussion of embodiments where the processing modules and other components of FIG. 30 are positioned within the surgical tool.

The external trial stimulator 50 generates controlled electrical pulses which, when delivered through the electrodes, apertures, or conductive stylet of the catheter, give rise to directional stimulation fields within the surrounding tissue. These stimulation fields are oriented based on the configuration of the catheter hardware and the selection of active channels by the stimulator.

In various embodiments, the catheter is connected to an external trial stimulator (also referred to herein as a pulse generator) that provides stimulation pulses to the electrodes or stylet of the catheter. The external trial stimulator includes field generator circuitry, channel switching circuitry, and directional mapping logic, while the catheter provides the physical interface for delivering directional stimulation fields to the patient.

In illustrative embodiments, the external trial stimulator includes a pulse generation and delivery module 62 configured to generate stimulation pulses with controlled amplitude, frequency, pulse width, and duty cycle. This module provides output signals to the electrodes, apertures, or conductive stylet of the catheter, thereby creating directional stimulation fields during intraoperative testing. The pulse generation and delivery module is capable of operating in monopolar, bipolar, or multipolar configurations, allowing a range of stimulation field geometries to be applied for mapping physiological responses and identifying a desirable therapeutic orientation.

The pulse generator module 62 may further include output multiplexing or current steering circuitry configured to route stimulation pulses to different directional stimulation features. In this way, the same stimulation waveform can be applied to multiple angular orientations, allowing the system to compare physiological responses across different field directions. The module therefore serves as the primary interface between the system's programmable stimulation parameters and the physical electrodes or conductive surfaces located on or within the catheter.

Communication between the pulse generator module 62 and other functional modules may be accomplished through a digital control bus or analog switching network. For example, the pulse generator module may be operatively coupled to a Channel Switching Module 64, which determines which electrode segment, aperture, or stylet conductor receives the active stimulation.

In some embodiments, the pulse generator module 62 may further incorporate safeguards to limit maximum delivered current or voltage, ensuring patient safety during intraoperative mapping. It may also include diagnostic channels that confirm electrode impedance, ensuring that a selected directional stimulation feature is functioning correctly before therapy is delivered. By coordinating closely with switching, feedback, and control modules, the pulse generator module enables the catheter to apply precise, orientation-specific stimulation fields and plays a central role in both mapping and therapy optimization workflows.

The stimulation system may further include a channel switching and orientation control module 64 for selecting which catheter electrode, aperture, or stylet region is energized at a given time. The module includes electronic switching circuitry, such as multiplexers, that enable rapid cycling through individual or combined electrode configurations. By iteratively cycling through directional options, the channel switching and orientation control module allows the clinician or system controller to compare on-target and off-target effects. In certain embodiments, this module interfaces with keyed or fiducial features of the catheter, thereby maintaining correspondence between the stimulation orientation and the physical angular orientation of the catheter.

In various embodiments, the channel switching & orientation control module 64 is configured to determine which of multiple directional stimulation features is energized during a mapping or therapy procedure. The module may be integrated into the catheter handle, housed in an external controller, or distributed between the two. It serves as the interface that selectively connects the output of the pulse generator module to one or more stimulation pathways, such as segmented electrodes disposed circumferentially around the catheter, apertures formed in the sheath, or conductive portions of a stylet located within the lumen.

The channel switching & orientation control module 64 may be implemented using multiplexing circuits, micro-relays, or solid-state switching elements that allow the system to cycle stimulation sequentially or simultaneously across multiple angular directions. For example, when four electrode segments are arranged at 0°, 90°, 180°, and 270° around the catheter body, the module may activate each electrode in turn while recording corresponding physiological responses. Similarly, when the catheter contains multiple apertures or stylet conductors, the module can selectively energize each pathway to produce directional stimulation fields at different orientations. By iteratively cycling through available options, the module enables the system to map on-target and off-target responses across orientations in a controlled and repeatable manner.

Communication between the channel switching & orientation control module and other components is coordinated by the system's control logic. The pulse generator module provides stimulation waveforms, which are routed through the switching module to a selected electrode or aperture. Simultaneously, a Feedback Processing Module monitors physiological responses, such as electromyography (EMG) activity or plethysmography, to determine whether the active orientation produces a therapeutic benefit. Based on this feedback, the control logic may instruct the channel switching & orientation control module to select a new electrode configuration, rotate through available channels, or lock onto the pathway corresponding to the optimal orientation.

In certain embodiments, the module may also integrate with fiducial tracking systems or handle-based encoders that record the angular position of the catheter. This integration allows the orientation of the selected stimulation pathway to be correlated with the physical position of the catheter in situ. By combining selective channel activation with positional awareness, the channel switching & orientation control module provides precise control over directional stimulation and ensures that the final implanted electrode can be aligned to replicate the orientation identified during intraoperative mapping.

In certain embodiments, the catheter system incorporates a Directional Mapping Logic or Intelligence Module 66 configured to manage the process of identifying an optimal/desirable stimulation orientation. This module provides the decision-making functionality that governs when to switch between directional stimulation features, how to compare the resulting physiological responses, and how to select the configuration that provides the greatest therapeutic benefit with the least adverse effect. The module may be implemented as embedded logic in the catheter handle, as firmware or software within an external controller, or distributed between both.

This module 66 records or displays the selected orientation so that the permanent electrode can be deployed in alignment with the identified field direction. In some embodiments, the decision and orientation recording module also stores the stimulation configuration in memory for subsequent programming into the implantable pulse generator (IPG), ensuring continuity between intraoperative mapping and long-term therapy delivery.

The directional mapping logic module receives input from the Feedback Processing Module 68, which may supply electromyography (EMG) signals, plethysmography data, respiratory airflow, blood oxygenation, or other physiological indicators of therapy efficacy. Based on these signals, the logic evaluates whether the currently energized directional stimulation feature meets predetermined criteria, such as exceeding an EMG amplitude threshold in a target muscle while maintaining sub-threshold activity in off-target muscles. If the criteria are not met, the module directs the Channel Switching & Orientation Control Module to activate a new electrode, aperture, or stylet pathway. In this manner, the module iteratively cycles through available directional options until an orientation is identified that produces a net positive therapeutic effect.

In more advanced embodiments, the directional mapping logic is configured as a closed-loop, automated process. Here, the module continuously receives real-time biofeedback, applies algorithms such as threshold detection, envelope detection, or statistical comparison, and automatically controls channel selection without requiring manual input from the physician. After an optimal orientation is found, the module may instruct the catheter to lock onto that configuration, record the fiducial orientation, and signal that the permanent therapy electrode should be deployed in alignment with the identified direction. This automation reduces procedural time, minimizes operator subjectivity, and improves the reproducibility of directional neuromodulation therapy.

The directional mapping logic module communicates bidirectionally with both the Pulse Generator Circuitry 112 and the Channel Switching & Orientation Control Circuitry 113. It may request new stimulation pulses at defined amplitudes or frequencies, instruct which stimulation pathway to energize, and receive confirmation of activation. At the same time, it monitors feedback data streams to compare therapy outcomes across orientations. By coordinating stimulation delivery, channel selection, and physiological evaluation, the module enables precise determination of the desirable stimulation orientation in a streamlined and automated fashion.

In various embodiments, the feedback acquisition module 68 id configured to collect physiological signals from the patient during delivery of directional stimulation. This module serves as the primary interface between the patient's physiological responses and the control logic that determines the optimal orientation of the stimulation field. The feedback acquisition module may be integrated into the catheter assembly, housed in an external controller, or distributed across both.

The feedback acquisition module 68 may collect signals from one or more physiological sensors. In some embodiments, electromyography (EMG) electrodes are placed on protrusor muscles such as the genioglossus to detect desired on-target activity, and on retrusor muscles such as the styloglossus or hyoglossus to detect off-target activation. In other embodiments, plethysmography sensors may be used to measure blood-oxygen saturation (SpO₂), airflow sensors may detect respiratory volume or flow, or pressure sensors may monitor airway patency. Additional feedback may be derived from surface EMG on associated muscle groups, from neuromonitoring systems, or from direct patient input such as verbal reports of sensation or discomfort.

Signals received by the feedback acquisition module 68 may be digitized, amplified, or filtered before transmission to the Directional Mapping Logic Module 66. For example, EMG signals may undergo envelope detection or thresholding to simplify downstream processing. In advanced embodiments, the feedback acquisition module may include onboard pre-processing circuits or microcontrollers that classify responses as on-target or off-target prior to delivery to the higher-level logic. This distributed architecture allows for faster response times and reduces the computational burden on the main controller.

The feedback acquisition module communicates directly with the Pulse Generator Module and the Channel Switching & Orientation Control Module 64 by providing real-time data during mapping. As stimulation is applied in different orientations, the feedback acquisition module continuously streams physiological responses to the control logic, which then determines whether to continue with the current configuration, switch to another directional pathway, or lock onto an identified optimal orientation. By enabling accurate capture of on-target and off-target effects, the feedback acquisition module provides the quantitative evidence required to achieve precise, minimally invasive directional neuromodulation therapy.

In some embodiments, the catheter system includes a Decision & Orientation Recording Module 65 configured to determine when the correct orientation of a directional stimulation field has been identified and to record or indicate this orientation for subsequent deployment of a permanent therapy electrode. This module functions as the bridge between intraoperative mapping and long-term therapy delivery, ensuring that the orientation which produces the most favorable therapeutic response can be reproduced with the indwelling electrode.

The decision and orientation recording module may operate by applying criteria to the feedback signals collected during directional mapping. For example, the module may classify a stimulation orientation as “correct” when on-target EMG activity exceeds a therapeutic threshold while off-target activity remains below an exclusion limit, or when blood-oxygen saturation (SpO₂) increases above baseline without signs of airway collapse. Once such a condition is met, the module signals that the optimal orientation has been achieved. In manual embodiments, this determination may be displayed to the physician for confirmation; in automated embodiments, the module may independently lock onto the orientation and mark it as the final deployment direction.

The module may also record the spatial orientation of the catheter using fiducial markers, angular encoders, or keyed mechanical couplings integrated into the catheter body or handle. This recorded orientation is correlated with the active directional stimulation feature to ensure consistency. For example, if the fiducial indicates that the therapeutic orientation corresponds to a 90° rotation of the cannula relative to a fixed anatomical landmark, the permanent electrode can then be deployed through the catheter lumen in that same 90° orientation. In some embodiments, the module may store orientation data electronically and transmit it to an external controller or implantable pulse generator.

Communication between the decision and orientation recording module and other modules is bi-directional. It receives feedback data from the Feedback Acquisition Module and decision criteria from the Directional Mapping Logic Module, then outputs an orientation lock signal to the Channel Switching & Orientation Control Module or to the operator interface. By preserving the identified orientation and linking it to fiducials or deployment markers, this module ensures that the implanted electrode will replicate the therapeutic field direction discovered during mapping, thereby eliminating the need for extensive post-deployment repositioning or reprogramming.

The data storage 63 can include one or more of non-transitory computer readable media, such as flash memory, solid state memory, magnetic memory, optical memory, cache memory, combinations thereof, and others. The data storage 63 can be configured to store executable instructions and data used for operation of the controller. In certain implementations, the data storage can include executable instructions that, when executed, are configured to cause the processor 58 to perform one or more functions.

In some examples, the network interface 206 can facilitate the communication of information between the controller and one or more other devices or entities over a communications network. For example, where the controller is included in an ambulatory (such as smart garment 110), the network interface 206 can be configured to communicate with a remote computing device such as a remote server or other similar computing device. The network interface 206 can include communications circuitry for transmitting data in accordance with a Bluetooth® wireless standard for exchanging such data over short distances to an intermediary device(s) (e.g., a base station, a “hotspot” device, a smartphone, a tablet, a portable computing device, and/or other devices in proximity of the wearable smart garment 110). The intermediary device(s) may in turn communicate the data to a remote server over a broadband cellular network communications link. The communications link may implement broadband cellular technology (e.g., 2.5G, 2.75G, 3G, 4G, 5G cellular standards) and/or Long-Term Evolution (LTE) technology or GSM/EDGE and UMTS/HSPA technologies for high-speed wireless communication. In some implementations, the intermediary device(s) may communicate with a remote server over a Wi-Fi™ communications link based on the IEEE 802.11 standard.

In certain implementations, the user interface 116 can include one or more physical interface devices such as input devices, output devices, and combination input/output devices and a software stack configured to drive operation of the devices. These user interface elements may render visual, audio, and/or tactile content. Thus the user interface 116 may receive input or provide output, thereby enabling a user to interact with the controller.

In some embodiments, the controller can also include at least one battery configured to provide power to one or more components integrated in the controller. The battery can include a rechargeable multi-cell battery pack. In one example implementation, the battery can include three or more lithium ion cells that provide electrical power to the other device components within the controller. The sensor interface 115 can be coupled to one or more sensors configured to monitor one or more physiological parameters of the patient. The sensors may be coupled to the controller via a wired or wireless connection.

Various embodiments of the invention may be implemented at least in part in any conventional computer programming language. For example, some embodiments may be implemented in a procedural programming language (e.g., “C”), or in an object oriented programming language (e.g., “C++”). Other embodiments of the invention may be implemented as preprogrammed hardware elements (e.g., application specific integrated circuits, FPGAs, programmable analog circuitry, and digital signal processors), or other related components.

In an alternative embodiment, the disclosed apparatus and methods (e.g., see the various flow charts described above) may be implemented as a computer program product for use with a computer system. Such implementation may include a series of computer instructions fixed either on a tangible, non-transitory medium, such as a computer readable medium (e.g., a diskette, CD-ROM, ROM, or fixed disk). The series of computer instructions can embody all or part of the functionality previously described herein with respect to the system.

Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies.

Among other ways, such a computer program product may be distributed as a removable medium with accompanying printed or electronic documentation (e.g., shrink wrapped software), preloaded with a computer system (e.g., on system ROM or fixed disk), or distributed from a server or electronic bulletin board over the network (e.g., the Internet or World Wide Web). In fact, some embodiments may be implemented in a software-as-a-service model (“SAAS”) or cloud computing model. Of course, some embodiments of the invention may be implemented as a combination of both software (e.g., a computer program product) and hardware. Still other embodiments of the invention are implemented as entirely hardware, or entirely software.

In some implementations, the processor 58 includes one or more processors (or one or more processor cores) that each are configured to perform a series of instructions that result in manipulated data and/or control the operation of the other components of the controller. In some implementations, when executing a specific process (e.g., cardiac monitoring), the processor 58 can be configured to make specific logic-based determinations based on input data received, and be further configured to provide one or more outputs that can be used to control or otherwise inform subsequent processing to be carried out by the processor 58 and/or other processors or circuitry with which processor 58 is communicatively coupled. Thus, the processor 58 reacts to specific input stimulus in a specific way and generates a corresponding output based on that input stimulus. In some example cases, the processor 58 can proceed through a sequence of logical transitions in which various internal register states and/or other bit cell states internal or external to the processor 58 may be set to logic high or logic low. As referred to herein, the processor 58 can be configured to execute a function where software is stored in a data store coupled to the processor 58, the software being configured to cause the processor 58 to proceed through a sequence of various logic decisions that result in the function being executed. The various components that are described herein as being executable by the processor 58 can be implemented in various forms of specialized hardware, software, or a combination thereof. For example, the processor can be a digital signal processor (DSP) such as a 24-bit DSP processor. The processor can be a multi-core processor, e.g., having two or more processing cores. The processor can be an Advanced RISC Machine (ARM) processor such as a 32-bit ARM processor. The processor can execute an embedded operating system, and include services provided by the operating system that can be used for file system manipulation, display & audio generation, basic networking, firewalling, data encryption and communications.

As used in this specification and the claims, the singular forms “a,” “an,” and “the” refer to plural referents unless the context clearly dictates otherwise. For example, reference to “the aperture” in the singular includes a plurality of apertures, and reference to “the contact” in the singular includes one or more contacts and equivalents known to those skilled in the art. Thus, in various embodiments, any reference to the singular includes a plurality, and any reference to more than one component can include the singular.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein.

It is, therefore, to be understood that the foregoing embodiments are presented by way of example only and that, within the scope of the appended claims and equivalents thereto, inventive embodiments may be practiced otherwise than as specifically described and claimed. Illustrative embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure. Disclosed embodiments, or portions thereof, may be combined in ways not listed above and/or not explicitly claimed. Thus, one or more features from variously disclosed examples and embodiments may be combined in various ways. For example, it is to be understood that the present disclosure contemplates that, to the extent possible, one or more features of any embodiment can be combined with one or more features of any other embodiment.

Various inventive concepts may be embodied as one or more methods, of which examples have been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Although the above discussion discloses various exemplary embodiments of the invention, it should be apparent that those skilled in the art can make various modifications that will achieve some of the advantages of the invention without departing from the true scope of the invention.

What is claimed is:

1. A system for delivering neuromodulation therapy, comprising:
a directional surgical tool having a lumen and configured to apply and/or aid in applying a directional stimulation field, the directional stimulation field having a primary field magnitude in a selected direction; and
a permanent electrode lead configured to be deployed through the lumen of the directional catheter in an orientation corresponding to a desirable stimulation direction determined as a function of the directional stimulation field.

2. The system of claim 1, wherein the directional surgical tool comprises a plurality of radial apertures configured to direct the stimulation field.

3. The system of claim 1, wherein the directional catheter comprises a plurality of electrodes disposed on an outer surface and a control mechanism to selectively activate one or more electrodes to change the direction of the stimulation field.

4. The system of claim 1, wherein the directional catheter further comprises a conductive stylet positioned within the lumen, the stylet having an exposed portion aligned with an aperture to deliver the directional stimulation field.

5. The system of claim 1, wherein the permanent electrode lead comprises a self-expanding paddle electrode configured to transition from a compressed state within the lumen to an expanded state providing a hemispherical stimulation field.

6. The system of claim 1, wherein the permanent electrode lead comprises a cylindrical lead having radially segmented electrodes to deliver a directional stimulation field.

7. The system of claim 1, further comprising alignment features on the directional catheter and the permanent electrode lead to maintain orientation during deployment, the alignment features comprising at least one of: fiducial markers, keyed connectors, or mechanical coupling elements.

8. The system of any of the above claims, wherein the permanent electrode lead comprises a self-expanding paddle electrode configured to transition from a compressed state within the lumen to an expanded paddle state when deployed from the catheter surgical tool.

9. A method of identifying a desirable stimulation direction and deploying an therapy electrode, comprising:
positioning a catheter having a lumen and directional stimulation capability feature relative to a target;
applying a directional stimulation field from the catheter in a first orientation;
receiving feedback indicative of whether the first orientation corresponds to a desirable stimulation direction;
adjusting the orientation of the directional stimulation field and repeating the applying and receiving steps until a desirable stimulation direction is identified;
deploying an electrode lead through the lumen of the catheter in an orientation corresponding to the desirable stimulation direction; and
removing the catheter while leaving the ?therapy/permanent? electrode lead in place.

10. The method of claim 9, wherein the electrode lead comprises a self-expanding paddle electrode configured to transition from a compressed state within the lumen to an expanded paddle shape when deployed.

11. The method of claim 10, wherein the self-expanding paddle electrode is biased to expand into a planar configuration that provides a hemispherical directional stimulation field when deployed.

12. The method of claim 9, wherein the electrode lead comprises a cylindrical lead having a plurality of radially segmented electrodes configured to deliver a directional stimulation field.

13. The method of claim 9, further comprising aligning the electrode lead with the desirable stimulation direction by using at least one of:
(a) radiopaque markers,
(b) visual or mechanical fiducials, and
(c) a keyed loading mechanism configured to maintain rotational orientation between the catheter and the electrode lead.

14. The method of claim 9, wherein the feedback comprises at least one of:
(a) an electrical signal measurement from a sensor,
(b) a mechanical displacement measurement, or
(c) a signal indicative of directional field coupling,
(d) a visible displacement of a motion element associated with the target
(e) an audible signal associated with the target.

15. A method of delivering a neuromodulation therapy electrode, the method comprising:
introducing a catheter into a patient, the catheter having a lumen configured to receive the therapy electrode and comprising at least one directional stimulation feature;
operating the directional stimulation feature to apply or assist in applying a directional stimulation field to identify a desirable stimulation orientation relative to a neural target;
delivering the therapy electrode through the lumen of the catheter in the desirable stimulation orientation; and
removing the catheter while leaving the therapy electrode implanted in the desirable stimulation orientation.

16. The method of claim 15, wherein the directional stimulation feature comprises one or more electrodes integrated on, within, or defined by the catheter wall.

17. The method of claim 15, wherein the directional stimulation feature comprises a conductive stylet or introducer core disposed within the lumen of the catheter.

18. The method of claim 15, wherein the directional stimulation feature comprises one or more apertures or windows defined in a wall of the catheter through which stimulation is directed.

19. The method of claim 15, wherein the therapy electrode comprises a segmented cylindrical electrode lead and is delivered such that one or more electrode segments align with the desirable stimulation orientation.

20. The method of claim 15, wherein the therapy electrode comprises a self-expanding paddle electrode that expands from a compressed configuration within the catheter lumen into the desirable stimulation orientation.
